(19) United States
(12) Patent Application Publication
(10) Pub. No.: US 2024/0404651 A1
(43) Pub. Date: Dec. 5, 2024

(54) KERNEL-ELASTIC AUTOENCODER

(71) Applicant: Yale University, New Haven, CT (US)

(72) Inventors: Haote Li, New Haven, CT (US); Yu Shee, New Haven, CT (US); Victor Batista, New Haven, CT (US)

(21) Appl. No.: 18/592,914

(22) Filed: Mar. 1, 2024

Related U.S. Application Data
(60) Provisional application No. 63/505,152, filed on May 31, 2023.

(51) Int. Cl. G16C 20/70; G06F 30/28

(52) U.S. Cl. CPC G16C 20/70; G06F 30/28

(57) ABSTRACT

Aspects of the present invention relate to a system including a transformer encoder with a compression layer, a transformer decoder with an expansion layer, the transformer encoder configured to transform one or more inputs into a control latent vector, a noise injection element configured to add noise to the control latent vector to create a noisy latent vector, a weighting element configured to add one or more weightings to the control latent vector to create an exact latent vector, and the transformer decoder configured to transform the noisy latent vector and exact latent vector into an output.

### CROSS-REFERENCE TO RELATED APPLICATIONS

This application claims priority to U.S. Provisional Application No. 63/505,152 filed on May 31, 2023, incorporated herein by reference in its entirety.

### STATEMENT REGARDING FEDERALLY SPONSORED RESEARCH OR DEVELOPMENT

This invention was made with government support under Grant No. 2124511 awarded by the National Science Foundation. The government has certain rights in the invention.

### BACKGROUND OF THE INVENTION

With the idea of generation, a variety of applications for drug discovery and Chemistry are made available [Maziarka et al., Journal of Cheminformatics, 2020; Moret et al., Nature Machine Intelligence, 2020; Skalic et al., Journal of chemical information and modeling, 2019; Wang et al., Nature Machine Intelligence, 2021]. Variational Autoencoder (VAE) [Diederik P Kingma and Max Welling. Auto-encoding variational bayes. arXiv preprint arXiv:1312.6114, 2013] stands out as a pioneer of generative models. Its versatility in molecule generation is explored by many works formats such as character [Gómez-Bombarelli et al., ACS central science, 2018], grammar [Kusner et al., In International conference on machine learning, pages 1945-1954. PMLR, 2017], and graph-based [Jin et al., In International conference on machine learning, PMLR, 2018] Variational Autoencoders (VAEs).

VAE differs from Autoencoder (AE) [Dana H Ballard, In Aaai, volume 647, pages 279-284, 1987] in that it achieves generation purpose by modeling data as probabilistic distributions. Whereas the goal of AE is to efficiently embed data into a low-dimensional space. However, the latent space of the AE often has regions that do not correspond to any encoded data and therefore sampling around encoded latent representations is not feasible. The loss function for sequence-to-sequence style VAE seeks to reconstruct cross-entropy terms as implemented in AE, while treating each latent vector as a distribution. Then by enforcing all latent vectors to prior distribution such as a Gaussian, decoding latent vectors sampled from this distribution gives results that resemble training data. Not limited by generation, VAEs' potentials are explored widely in molecule property prediction and optimizations which are important steps in computational drug discovery.

VAE performance for molecule generation is measured in terms of novelty (N), uniqueness (U), validity (V), and reconstruction (R). Though all VAE models aim to achieve the highest metrics, they are constrained by a trade-off between the NUV and reconstruction. A model that reconstructs well might not be able to achieve high NUV and vice versa. Whichever gets closer to the optimum, the other will suffer. For example, in the case of molecule generation, if a VAE model is capable of reconstructing the input unambiguously, inferencing on latent vectors that the decoder has not seen before would be unpractical, being less likely to produce valid outputs. This trade-off leads to additional concerns, especially since these models lack the ability to perform precise optimization around a target molecule [Madhawa et al., arXiv preprint arXiv:1905.11600, 2019]. Being able to reconstruct is important because it ensures success in interpolating between molecules in latent representation as well as sampling close molecular scaffolds for a given target.

Graph-based VAE methods take the lead in chemical validity [Jin et al., In International conference on machine learning, pages 2323-2332. PMLR, 2018; Jin et al., In International conference on machine learning, pages 4839-4848. PMLR, 2020]. This is because the molecules are represented in graphs of motifs and these motifs explicitly enforce grammar rules onto the molecules, so the generated molecules are all valid. However, this is not the case without checking for grammar; during testing, if certain motifs do not exist in the training dataset, the model would not be able to reconstruct or sample similar molecules.

Flow-based generative models, on the other hand, excel in the reconstruction by memorizing the training dataset [Zang et al., In Proceedings of the 26^{th} ACM SIGKDD International Conference on Knowledge Discovery & Data Mining, pages 617-626, 2020; Luo et al., In International Conference on Machine Learning, pages 7192-7203. PMLR, 2021]. The models consist of invertible maps from input data to latent space with the same dimension so the model can map latent vectors back to the input molecules exactly. Nevertheless, same-dimensional latent representations are criticized for not being able to capture features that are important and tend to overfit. Out-of-distribution problems could arise during the sampling process and the reconstruction on the testing dataset remains a concern [Nalisnick et al., arXiv preprint arXiv:1810.09136, 2018].

Disclosed herein is a novel self-supervised generative kernel-elastic autoencoder that enhances the performance of traditional VAE by designing both modified maximum mean discrepancy and weighted reconstruction loss functions. The disclosed system has the potential to provide substantial contributions to generative models (e.g. molecular design and optimization). Thus, there is a need in the art to address long-standing challenges of generative models such as achieving superior generation and reconstruction performances simultaneously. The present invention satisfies that need.

### SUMMARY OF THE INVENTION

Aspects of the present invention relate to a system including a transformer encoder with a compression layer, a transformer decoder with an expansion layer, the transformer encoder configured to transform one or more inputs into a control latent vector, a noise injection element configured to add noise to the control latent vector to create a noisy latent vector, a weighting element configured to add one or more weightings to the control latent vector to create an exact latent vector, and the transformer decoder configured to transform the noisy latent vector and exact latent vector into an output.

In some embodiments, the one or more inputs is selected from one or more condition-scaled embedding vectors, one or more Simplified Molecular Input Line Entry System (SMILES) tokens, one or more SMILES Arbitrary Target Specification (SMARTS) tokens, one or more center-labelled products (CLP), reacting sites, reacting centers, or one or more reaction center labeled target molecules or compounds.

In some embodiments, the output is selected from one or more Simplified Molecular Input Line Entry System (SMILES) tokens, one or more SMILES Arbitrary Target Specification (SMARTS) tokens, one or more synthesis pathways, one or more retrosynthesis pathways, one or more labelled molecules or compounds, one or more templates, one or more reaction templates, one or more site-specific templates (SST).

In some embodiments, the system further comprises one or more condition-scaled embedding vectors configured to attach one or more conditions to the output of the transformer decoder. In some embodiments, the one or more conditioned-scaled embedding vectors are selected from molecule properties, SMILES tokens, positional embeddings, reacting sites, reaction centers, positional embedding for reacting sites or reaction centers, or molecular transformation sites.

In some embodiments, the transformer decoder is configured to pass the output through a linear layer, and softmax the output, to produce one or more output distribution probabilities. In some embodiments, the transformer system is further configured to calculate a distance between a control latent vector used to generate a first output and a control latent vector used to generate a second output to produce a measured distance between the first and second outputs.

Aspects of the present invention relate to method for retrosynthetic planning having the steps of providing one or more target molecules, specifying one or more reaction centers on the one or more target molecules, comparing the one or more target molecules to a database of reference reactions, measuring a similarity between at least one of the one or more target molecules and a molecule in the reference reactions, and generating one or more site-specific templates based on the measured similarity.

In some embodiments, the noise is gaussian noise. In some embodiments, the transformer decoder and the latent space comprises a lambda-delta loss function.

In some embodiments, the transformer encoder is configured to accept one or more positional embedding inputs for reaction centers. In some embodiments, the output comprises a reaction template.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1A shows an exemplary transformer-based architecture (in some examples referred to as Kernel-Elastic Autoencoder (KAE)) with 6 transformer encoder components (grey-red) and 6 decoder components (grey-red-blue). The gradient color represents the mixing of information from different sources. Condition is represented as grey and encoder and decoder inputs as red and blue. The vector after the compression layers is referred to as the latent vector. During training, a noise e is added before the expansion which produces the encoder output. If the Conditional Autoencoder (CKAE) is used, condition-scaled embeddings are concatenated to both the latent vector after noise and to the encoder output. KAE is trained as all conditions being zero. The decoder performs self-attention on the output sequences and obtains information from the encoder output by performing encoder-decoder attention. The decoder output is finally passed through a linear layer and softmaxed to produce the output token probabilities for each character in the size T dictionary.

FIG. 1B shows a pictorial illustration of an exemplary training procedure. Latent information from the encoder is passed to the decoder twice. One pass being AE-like without noise and the other pass being Variational Autoencoder-like (VAE) with added noise. λ controls the shape of the latent vector distribution and the relative weights between the maximum mean discrepancy (MMD) term and the Cross-Entropy loss. 6 controls the relative weights of between the AE and VAE objective.

FIG. 2 depicts an illustrative computer architecture for a computer for practicing the various embodiments of the invention.

FIGS. 3A, 3B, 3C, and 3D show a performance comparison of the models trained with modified MMD (m-MMD) loss, standard MMD (s-MMD) loss, and Kullback-Leibler (KL) divergence loss: 901 KL; 902 m-MMD; 903 s-MMD; 904 m-MMD, no noise; 905 s-MMMD, no noise. The model labelled as KL has one extra layer that estimates the standard deviation of each latent vector. The models labelled with m-MMD are trained with the loss [symbol] +m−MMD(λ=1), s-MMD with [symbol] +sMMD(λ=1), and KL with [symbol] . “No noise” means no noise is added to the latent vectors during training. Validity, uniqueness, and novelty are calculated at the end of each epoch from 1 k randomly sampled latent vectors. The reconstruction rate is calculated using 1 k molecules from the validation set.

FIGS. 4A, 4B, 4C, and 4D show the performance comparison of the models trained with different λ values (and δ=1) using m-MMD loss: 906 L1D1; 907 L2D1; 908 L3D1; 909 L3.5D1; 910 L4D1. Validity, uniqueness, and novelty are calculated at the end of each epoch using 1000 randomly generated molecules from each of the models. And the reconstruction rate is calculated using 1000 molecules from the validation set. Note that LxDy in the legend means that the model is trained with λ=x and δ=y. For example, the model labelled with L3D1 is trained with [symbol] (λ=3, δ=1).

FIGS. 5A through 5F show molecules found by sampling from a 0.1-SD Gaussian distribution centered around a specific latent vector 10 times. The noised vectors are decoded with different beam sizes each time. FIGS. 5A, 5B, 5C, 5D, 5E, and 5F show all the unique molecules found at these beam sizes. FIGS. 5A, 5B, and 5C shows only one unique molecule (the original encoded molecule) is found with the beam size of one despite the added noise. FIGS. 5D, 5E, and 5F shows out of the ten samples, seven different molecules are found at the beam size of two.

FIG. 6 shows an exemplary repositioning process. A [symbol] is selected around zᵢ if both the produced molecule is within the allowed threshold (σ) for similarity and the property under optimization is improved. The next repetition of the search is performed around [symbol] . By doing repositioning, the search space is expanded for molecules with little improvements in the condition search.

FIG. 7 shows the mean values produced from the model (blue dots) by sampling 1k vectors in the latent space at different asked conditions. The ground truth values are marked as a black line. The dataset's underlying distribution for the corresponding properties is rendered as a histogram background.

FIGS. 8A through 8D show plots for m-MMD and s-MMD latent space compared to standard Gaussian distribution. For all subplots, the latent vectors are obtained by passing 10k ZINC250k molecules to the encoder. They are then transformed under the same principal components extracted from the standard Gaussian distribution. FIG. 8A shows the m-MMD results showing all latent vectors are well-incorporated in the Gaussian. FIG. 8B shows the s-MMD loss makes the latent vectors more scattered relative to the Gaussian. The model is less likely to be able to obtain valid output by sampling from the Gaussian. For FIG. 8C and FIG. 8D, the latent noise used during training is added to visualize the actual vectors passed into decoder in the training process. FIG. 8A shows the m-MMD-resulted latent space in orange dots. Most latent vectors are well-incorporated in the standard Gaussian distribution in the principal components reference frame. FIG. 8B shows the s-MMD-resulted latent space in orange dots. The encoded vectors from the s-MMD model are more scattered than in the m-MMD case. FIG. 8C shows the m-MMD-resulted latent space with latent noise added in orange dots. FIG. 8D shows the s-MMD-resulted latent space with latent noise added in orange dots.

FIGS. 9A, 9B, 9C, and 9D show the results for the performance comparison of the models trained with different sigma values using modified MMD loss: 911 m-MMD 2ss=5e-4×E; 912 m-MMD 2ss=5e-3×E; 913 m-MMD 2ss=5e-2×E; 914 m-MMD 2ss=5e-1×E. Note that 2ss (2 sigma squared) in the legend represents the value used for 2σ² in Equation 3 and E is the embedding dimension. Validity, uniqueness, and novelty are calculated at the end of each epochs using 1000 randomly generated molecules from each of the models. And the reconstruction rate is calculated using 1000 molecules from the validation set. FIG. 9A shows the validity of the generated molecules. FIG. 9B shows the uniqueness of the generated molecules. FIG. 9C shows the novelty of the generated molecules. FIG. 9D shows the reconstruction rate of the molecules from the validation set.

FIGS. 10A, 10B, 10C, and 10D show the performance comparison of the models trained with different sigma values using standard MMD loss: 911 m-MMD 2ss=5e-4×E; 912 m-MMD 2ss=5e-3×E; 913 m-MMD 2ss=5e-2×E; 914 m-MMD 2ss=5e-1×E. Note that 2ss (2 sigma squared) in the legend represents the value used for 2σ² in Equation 3 and E is the embedding dimension. Validity, uniqueness, and novelty are calculated at the end of each epochs using 1000 randomly generated molecules from each of the models. And the reconstruction rate is calculated using 1000 molecules from the validation set. FIG. 10A shows the validity of the generated molecules. FIG. 10B shows the uniqueness of the generated molecules. FIG. 10C shows the novelty of the generated molecules. FIG. 10D shows the reconstruction rate of the molecules from the validation set.

FIGS. 11A, 11B, 11C, and 11D show the performance comparison of the models trained with different δ values (and λ=1) using modified MMD loss and KL loss: 915 L1D-1; 916 L1D0; 917 L1D1; 918 L1D2; 919 L1D4; 920 KL L1D1. Validity, uniqueness, and novelty are calculated at the end of each epoch using 1000 randomly generated molecules from each of the models. And the reconstruction rate is calculated using 1000 molecules from the validation set. Note that LxDy in the legend means that the model is trained with λ=x and δ=y. For example, the model labelled with L1D-1 is trained with [symbol] (λ=1, δ=−1). FIG. 11A shows the validity of the generated molecules. FIG. 11B shows the uniqueness of the generated molecules. FIG. 11C shows the novelty of the generated molecules. FIG. 11D shows the reconstruction rate of the molecules from the validation set.

FIGS. 12A, 12B, 12C, and 12D show an exemplary reaction template for the SMARTS string: [C: 5]−[O; H0; D2; +0:6]−[S; H0; D4; +0:1](−[C: 2])(=[O; D1; H0:3])=[O; D1; H0:4]>>Cl−[S; H0; D4; +0:1](−[C: 2])(=[O; D1; H0:3])=[O; D1; H0:4]. [C: 5]−[OH; D1; +0:6]. The product CCS(═O)(═O)OCCBr can be passed into the template to obtain the reaction SMARTS string: CCS(═O)(═O)Cl. OCCBr>>CCS(═O)(═O)OCCBr.

FIGS. 13A, 13B, 13C, and 13D show an exemplary conversion from a template to an intramolecular template. The original template is [C: 3]−[C; H0; D3; +0:2](=[O; H0; D1; +0:1])−[CH2; D2; +0:4]−[C: 5]>>C−[O; H0; D2; +0:1]−[C; H0; D3; +0:2](═O)—[C: 3].I−[CH2; D2; +0:4]−[C: 5] and the converted template is [C: 3]−[C; H0; D3; +0: 2](=[O; H0; D1; +0: 1])−[CH2; D2; +0: 4]−[C: 5]>>(C−[O; H0; D2; +0: 1]−[C; H0; D3; +0: 2](═O)−[C: 3].I−[CH2; D2; +0: 4]−[C: 5]).

FIGS. 14A & 14B show the data scalability of the exemplary product-masking model. Note that the validity here also means that the templates are unique. Trained for an equivalent-epoch means that the model is trained with the same number of updates as 1 epoch of full-dataset training. FIG. 14A shows a unique and valid rate of models with different training dataset size at 1 equivalent-epoch. FIG. 14B shows a unique and valid rate of models trained with different training dataset size at different equivalent-epochs.

FIGS. 15A through 15C show exemplary retrosynthetic routes proposed by commercial platforms and CKAE. FIG. 15A shows no results from Reaxys. FIG. 15B shows one of the routes proposed by SciFinder found in 40 minutes. FIG. 15C shows one of the routes proposed by CKAE found in 5 minutes.

FIGS. 16A through 16C show common machine learning methods for retrosynthesis as well as an exemplary method disclosed herein. FIG. 16A is a diagram showing that reactants and templates can be selected and generated based on a target compound using different machine learning models. In one example, template generation is used in the disclosed method. FIG. 16B is a diagram showing that latent space is incorporated in one of the models in the disclosed method according to aspects of the present invention. In some embodiment, the method comprises sampling in latent space in order to give different reaction templates. FIG. 16C shows the results of the disclosed method compared to a previous method, displaying a reduction in synthesis steps for a key intermediate for active pharmaceutical ingredients (API).

FIGS. 16D & FIG. 16E show Model A and Model B workflows and performance according to aspects of the present invention. FIG. 16D shows that Model B has reaction center embedding and does not have center-labeled products in the output. FIG. 16E shows the USPTO-Full Top-K accuracy performance for previous models compared to the models using the disclosed method.

FIGS. 17A & FIG. 17B show an exemplary interpolation of templates in the latent space of Model C, and reactants from Model C outputs, according to various aspects of the present invention. FIG. 17A is a diagram of an exemplary interpolation method showing that the intermediates of the top and bottom latent representations are decoded. FIG. 17B shows an aspect of the disclosed method involving selecting reactants for 2-, 3-, 4-substituted cyclohexanone derivatives as target compounds.

FIGS. 18A through 18C shows an exemplary retrosynthesis tree for compound 1 and its experimental procedure. FIG. 18A is a diagram showing that a synthesis route is selected from the retrosynthesis tree generated by Model B. FIG. 18B shows an exemplary reference found with Model C for the allylation step in the disclosed method. FIG. 18C shows a chemical synthesis reaction of the related experimental procedure for the selected route.

FIGS. 19A through 19D show exemplary reaction templates showing RDChiral Template vs Site-Specific Template. FIG. 19A shows an exemplary Reaction Example. FIG. 19B shows an exemplary RDChiral Template. FIG. 19C shows an exemplary Site-Specific Template. FIG. 19D shows a resultant Center-Labeled Product.

FIG. 20A through 20D show an exemplary reaction wherein a site-specific template requires a product/target compound with reaction centers labeled in order to get the reaction smart string: CCCCC[C@H](O)C═CC1C═CC(═O)C1CC═CCCCC(═O)O>>CCCCC[C@H](O)C═CC1CCC(═O)C1CC═CCCCC(═O). FIG. 20A shows an exemplary Reaction Example. FIG. 20B shows an exemplary RDChiral Template. FIG. 20C shows an exemplary Site-Specific Template according to aspects of the present invention. FIG. 20D shows a resultant Center-Labeled Product according to aspects of the present invention.

FIG. 21 shows exemplary model architectures of the generative models for retrosynthesis planning according to aspects of the present invention, comprising columns 501, 502 and 502. Column 501 comprises Model A, which is a deterministic generative model that takes in target products and output site-specific templates and labeled products. Column 502 comprises Model B, a variant of Model A, incorporating positional embeddings for conditioning on specific reacting sites. Column 503 comprises Model C, a sampling generative model based on the conditional kernel-elastic autoencoder (CKAE) method according to aspects of the present invention.

FIG. 22 presents a compilation of the top 5 references for the allylation step depicted in FIG. 18B. The site-specific templates are the same for these 10 references. Therefore, the products of these reactions are the primary determinant for the ranking (latent distance) in this particular case.

FIGS. 23A through 23D show a visualization of the encoder-decoder-attention obtained from the product: CC(═O)c1ccc(Cn2ncc(NC(═O)c3nc(C)oc3-c3cccc(C(F)(F)F)c3)n2)o1. FIG. 23A shows the Encoder Input Product (centers are from decoder output) according to aspects of the present invention. FIG. 23B shows the Decoder Output Template according to aspects of the present invention. FIG. 23C shows the Corresponding Reaction. FIG. 23D shows the encoder-Decoder Attention Matrix according to aspects of the present invention.

FIG. 24 depicts the synthesis of (R)-2-Allyl-2-methylcyclohexan-1-one [D. C. Behenna et al., Journal of the American Chemical Society, 2004].

FIG. 25 depicts the synthesis of (R)-2-(1-Methyl-2-oxocyclohexyl)acetaldehyde

FIG. 26 depicts the synthesis of (R)-2-ethynyl-2-methylcyclohexan-1-one.

FIGS. 27A through 27F show the results for exemplary queries. FIGS. 27A and 27B are the queries for ¹H and ¹³C NMR of (R)-2-Allyl-2-methylcyclohexan-1-one, respectively. FIGS. 27C and 27D are the queries for 1H and 13C NMR of (R)-2-(1-Methyl-2-oxocyclohexyl)acetaldehyde, respectively. FIGS. 27E and 27F are the queries for 1H and 13C NMR of ®-2-ethynyl-2-methylcyclohexan-1-one, respectively.

### DETAILED DESCRIPTION

It is to be understood that the figures and descriptions of the present invention have been simplified to illustrate elements that are relevant for a clear understanding of the present invention, while eliminating, for the purpose of clarity, many other elements found in related systems and methods. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, 6 and any whole and partial increments therebetween. This applies regardless of the breadth of the range.

### Kernel-Elastic Autoencoder for Template Generative Retrosynthetic Pathways

Aspects of the present invention relate to a system and method for improving existing generative models. In some embodiments, the invention provides a method for discovery of retrosynthesis pathways to generate a target chemical product.

In some embodiments, the disclosed system and method improves upon existing generative models by ensuring accurate reconstruction as well as rapid generation of a large set of diverse reaction pathways.

Existing generative models for retrosynthesis are not based on reaction templates, and use the whole reaction strings for input/output definition. In contrast, the novel disclosed method (in some examples, referred to as a kernel) focuses only on the encoding/decoding of the molecular changes in substructures, allowing it to explore a much larger space than reactant-encoding-only models. One significant technical advantage of the novel disclosed kernel is an exemplary generative method based on templates of chemical reactions for inputs and outputs. The disclosed method eliminates the redundant hyperspace that would be required to encode chemical reactions with complete definition of molecular structures.

Another technical advantage is the use of a generative method for solving the searching problem, rather than implementing a prediction algorithm based on a traditional deterministic procedure. An advantage of the generative method is that both known and novel reactions are included in the unlimited search space, significantly expanding the range of plausible solutions beyond the capabilities of deterministic methods.

Aspects of the present invention relate to a novel system for a generative transformer architecture. In some embodiments, the system comprises a novel kernel for a generative transformer architecture. In some embodiments, the novel kernel is used to define the loss function of a generative transformer architecture, in some examples referred to as Kernel Autoencoder (KAE). In other examples, the novel kernel is referred to as Kernel-Elastic Autoencoder (KAE) and/or Anisotropic Kernel Model (AKM). In some embodiments, the resulting kernel provides a modified version of a maximum mean discrepancy loss.

In some embodiments, the generative transformer kernel is based on a loss function and a beam search procedure that ensures accurate reconstruction as well as diverse generation of templates of chemical reaction pathways and reactants corresponding to a target molecular product. In some embodiments, the reactants corresponding to the generated templates are iteratively processed to generate a complete multistep retrosynthetic pathway.

In some embodiments, the resulting kernel exhibits state-of-the-art generative performance, while a lambda-delta (LD) loss function ensures accurate reconstruction. The loss function affects the output and regularize the latent space. In some embodiments, the novel kernel is trained to generate reaction templates rather than complete reactions.

In some embodiments, a masking strategy is used to mask exclusively the product side of the template, rather than implementing random masking schemes. In some embodiments, a beam search procedure was implemented for both sampling and multi-step generation. In some embodiments, an encrypted code was used to confirm the output was produced by the disclosed novel kernel. Validation of the proposed pathways, as well as estimated cost can be obtained by literature reports of the individual reaction steps that constitute the predicted reaction pathway.

Aspects of the present invention relate to a generative retrosynthetic model. The disclosed generative retrosynthetic model can provide significant economic advantages in applications to research and develop drugs and fine chemicals by significantly reducing the time for discovery and development of synthetic procedures, and by reducing the cost of synthetic procedures based on reaction pathways with a minimum number of steps.

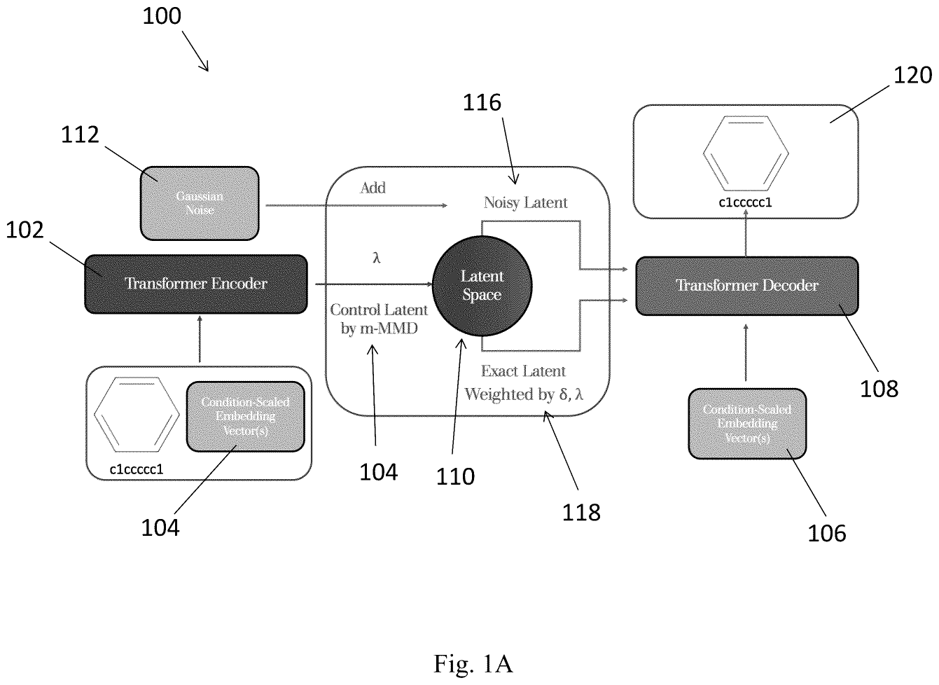

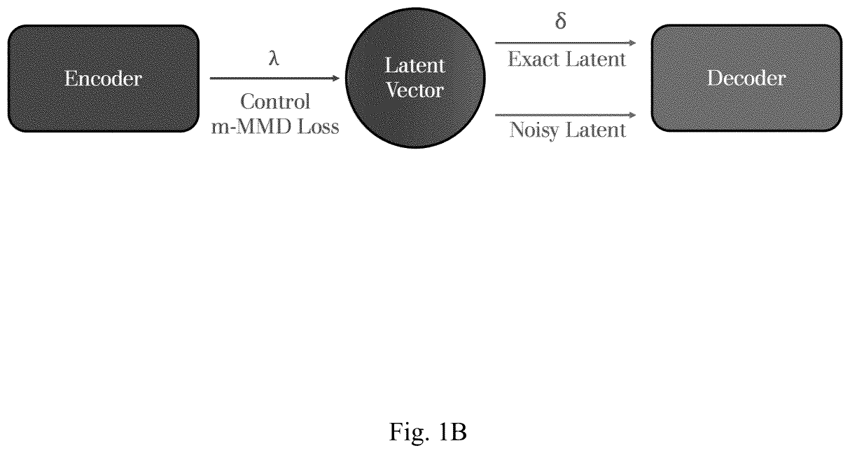

Referring now to FIG. 1A, shown is an exemplary transformer-based system 100 comprising 6 transformer encoder components (grey-red) and 6 decoder components (grey-red-blue). It should be noted that the gradient color in latent space represents the mixing of information from different sources. The condition is represented as grey, and encoder and decoder inputs as red and blue. The vector after the compression layers is referred to as the latent vector. In some embodiments, during training, a noise e is added before the expansion which produces the encoder output. If the conditional system is used, condition-scaled embeddings are concatenated to both the latent vector after noise and to the encoder output. The disclosed system is trained as all conditions being zero. The decoder performs self-attention on the output sequences and obtains information from the encoder output by performing encoder-decoder attention. The decoder output is finally passed through a linear layer and softmaxed to produce the output token probabilities for each character in the size T dictionary.

FIG. 1A depicts an exemplary system 100 architecture with the option for adding conditions. In some embodiments, system 100 comprises a transformer encoder 102 comprising a compression layer, and a transformer decoder 108 comprising an expansion layer. In some embodiments, transformer encoder 102 and/or transformer decoder 108 further comprises one or more embedding layers. In some embodiments, system 100 further comprises a latent space 110 wherein a Gaussian noise 112 may be added. In some embodiments, transformer encoder 102 produces a latent vector λ 114, which when combined in latent space 110, forms a noisy latent vector 116 and an exact latent vector 118. In some embodiments, noisy latent vector 116 and exact latent vector 118 are compressed into transformer decoder 108, wherein transformer decoder 108 produces an output 120.

In some embodiments, attention operations are performed with four heads. In some embodiments, for each input, with specified padding tokens, both the source and target masks are made to prevent the model from attending to paddings during training.

In some embodiments, SMILES tokens 104 and 106 are passed through encoder 102 and decoder 108 embedding layers that transform each token into an E-dimensional vector where E is the embedding size and is equal to 128 for all implementations disclosed herein. In some embodiments, these vectors are added to the encoder and decoder specific E-dimensional positional embeddings.

In some embodiments, system 100 is conditional (e.g., Conditional KAE (CKAE)), and additional embeddings are used just for attached condition(s) such as different molecule properties. In some embodiments, CKAE's condition-scaled embeddings are concatenated to both the input of the encoder and the latent representation along the sequence length dimension, allowing the model to generate molecules by interpolating and extrapolating with asked properties as conditions.

In some embodiments, the encoder input is processed by the Transformer encoder followed by a compression in the sequence length dimension. In some embodiments, this latent vector of 10×E dimensions (a 10-dimensional compressed sequence length by E embedding size) is injected with noise during training. In the case of CKAE, it is concatenated with the property-scaled embedding vector condition. In some embodiments, this processed latent vector is mapped back to M×E dimensions by the expansion layer where M is the maximum sequence length in the relevant dataset. In some embodiments, this vector is treated as the final encoder output, fed into the decoder without supplying encoder masks.

In some embodiments, each decoder layer attends to the encoder outputs through the encoder-decoder muti-head attention operations. In some embodiments, outputs are contracted by a linear layer along the embedding dimension to produce a T-dimensional vector per token. In some embodiments, this T-dimensional character is then softmaxed, and interpreted as a probability distribution (P) for each possible character (c).

### Computing Device

In some aspects of the present invention, software executing the instructions provided herein may be stored on a non-transitory computer-readable medium, wherein the software performs some or all of the steps of the present invention when executed on a processor.

Aspects of the invention relate to algorithms executed in computer software. Though certain embodiments may be described as written in particular programming languages, or executed on particular operating systems or computing platforms, it is understood that the system and method of the present invention is not limited to any particular computing language, platform, or combination thereof. Software executing the algorithms described herein may be written in any programming language known in the art, compiled, or interpreted, including but not limited to C, C++, C#, Objective-C, Java, JavaScript, MATLAB, Python, PHP, Perl, Ruby, or Visual Basic. It is further understood that elements of the present invention may be executed on any acceptable computing platform, including but not limited to a server, a cloud instance, a workstation, a thin client, a mobile device, an embedded microcontroller, a television, or any other suitable computing device known in the art.

Parts of this invention are described as software running on a computing device. Though software described herein may be disclosed as operating on one particular computing device (e.g. a dedicated server or a workstation), it is understood in the art that software is intrinsically portable and that most software running on a dedicated server may also be run, for the purposes of the present invention, on any of a wide range of devices including desktop or mobile devices, laptops, tablets, smartphones, watches, wearable electronics or other wireless digital/cellular phones, televisions, cloud instances, embedded microcontrollers, thin client devices, or any other suitable computing device known in the art.

Similarly, parts of this invention are described as communicating over a variety of wireless or wired computer networks. For the purposes of this invention, the words “network”, “networked”, and “networking” are understood to encompass wired Ethernet, fiber optic connections, wireless connections including any of the various 802.11 standards, cellular WAN infrastructures such as 3G, 4G/LTE, or 5G networks, Bluetooth®, Bluetooth® Low Energy (BLE) or Zigbee® communication links, or any other method by which one electronic device is capable of communicating with another. In some embodiments, elements of the networked portion of the invention may be implemented over a Virtual Private Network (VPN).

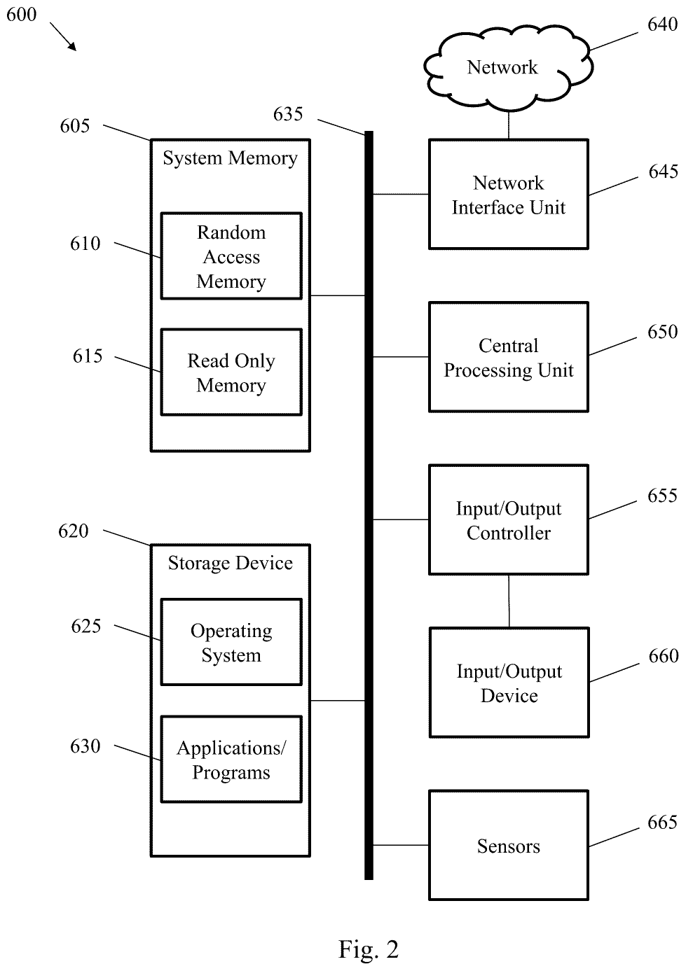

FIG. 2 and the following discussion are intended to provide a brief, general description of a suitable computing environment in which the invention may be implemented. While the invention is described above in the general context of program modules that execute in conjunction with an application program that runs on an operating system on a computer, those skilled in the art will recognize that the invention may also be implemented in combination with other program modules.

Generally, program modules include routines, programs, components, data structures, and other types of structures that perform particular tasks or implement particular abstract data types. Moreover, those skilled in the art will appreciate that the invention may be practiced with other computer system configurations, including hand-held devices, multiprocessor systems, microprocessor-based or programmable consumer electronics, minicomputers, mainframe computers, and the like. The invention may also be practiced in distributed computing environments where tasks are performed by remote processing devices that are linked through a communications network. In a distributed computing environment, program modules may be located in both local and remote memory storage devices.

FIG. 2 depicts an illustrative computer architecture for a computer 600 for practicing the various embodiments of the invention. The computer architecture shown in FIG. 2 illustrates a conventional personal computer, including a central processing unit 650 (“CPU”), a system memory 605, including a random-access memory 610 (“RAM”) and a read-only memory (“ROM”) 615, and a system bus 635 that couples the system memory 605 to the CPU 650. A basic input/output system containing the basic routines that help to transfer information between elements within the computer, such as during startup, is stored in the ROM 615. The computer 600 further includes a storage device 620 for storing an operating system 625, application/program 630, and data.

The storage device 620 is connected to the CPU 650 through a storage controller (not shown) connected to the bus 635. The storage device 620 and its associated computer-readable media provide non-volatile storage for the computer 600. Although the description of computer-readable media contained herein refers to a storage device, such as a hard disk or CD-ROM drive, it should be appreciated by those skilled in the art that computer-readable media can be any available media that can be accessed by the computer 600.

By way of example, and not to be limiting, computer-readable media may comprise computer storage media. Computer storage media includes volatile and non-volatile, removable and non-removable media implemented in any method or technology for storage of information such as computer-readable instructions, data structures, program modules or other data. Computer storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

According to various embodiments of the invention, the computer 600 may operate in a networked environment using logical connections to remote computers through a network 640, such as TCP/IP network such as the Internet or an intranet. The computer 600 may connect to the network 640 through a network interface unit 645 connected to the bus 635. It should be appreciated that the network interface unit 645 may also be utilized to connect to other types of networks and remote computer systems.

The computer 600 may also include an input/output controller 655 for receiving and processing input from a number of input/output devices 660, including a keyboard, a mouse, a touchscreen, a camera, a microphone, a controller, a joystick, or other type of input device. Similarly, the input/output controller 655 may provide output to a display screen, a printer, a speaker, or other type of output device. The computer 600 can connect to the input/output device 660 via a wired connection including, but not limited to, fiber optic, Ethernet, or copper wire or wireless means including, but not limited to, Wi-Fi, Bluetooth, Near-Field Communication (NFC), infrared, or other suitable wired or wireless connections.

As mentioned briefly above, a number of program modules and data files may be stored in the storage device 620 and/or RAM 610 of the computer 600, including an operating system 625 suitable for controlling the operation of a networked computer. The storage device 620 and RAM 610 may also store one or more applications/programs 630. In particular, the storage device 620 and RAM 610 may store an application/program 630 for providing a variety of functionalities to a user. For instance, the application/program 630 may comprise many types of programs such as a word processing application, a spreadsheet application, a desktop publishing application, a database application, a gaming application, internet browsing application, electronic mail application, messaging application, and the like. According to an embodiment of the present invention, the application/program 630 comprises a multiple functionality software application for providing word processing functionality, slide presentation functionality, spreadsheet functionality, database functionality and the like.

The computer 600 in some embodiments can include a variety of sensors 665 for monitoring the environment surrounding and the environment internal to the computer 600. These sensors 665 can include a Global Positioning System (GPS) sensor, a photosensitive sensor, a gyroscope, a magnetometer, thermometer, a proximity sensor, an accelerometer, a microphone, biometric sensor, barometer, humidity sensor, radiation sensor, or any other suitable sensor.

### Template Generation

Disclosed herein is a novel generation-based method for retrosynthesis planning that represents a distinct category, sometimes referred to herein as template generation. In some embodiments, the disclosed system and method comprise template generation models that employ a Sequence-to-Sequence (S2S) architecture, and that are trained to translate product information into reaction templates, as opposed to generating reactants. This system and method transcends the limitations of template selection-based approaches, enabling the discovery of novel reaction rules and expanding the scope of retrosynthesis planning. In some aspects, the disclosed system and method combine generated reaction templates and the “RunReactants” function from RDKit, and offer an efficient means to swiftly identify templates that yield grammatically coherent reactants from given products. This facilitates the exploration of previously uncharted chemical reactions and pathways.

One of the major benefits of using the reaction template is the ease of checking reaction validity. During the transformation of a reaction template, the product is guaranteed to be converted to the reactant with exact matching of atoms indices and relevant functional groups from the description of template. In comparison to reactant generative models, this benefit greatly reduces the uncertainty in the produced reactants which might not correspond to any known reactions or have key atom mismatches due to problems during decoding.

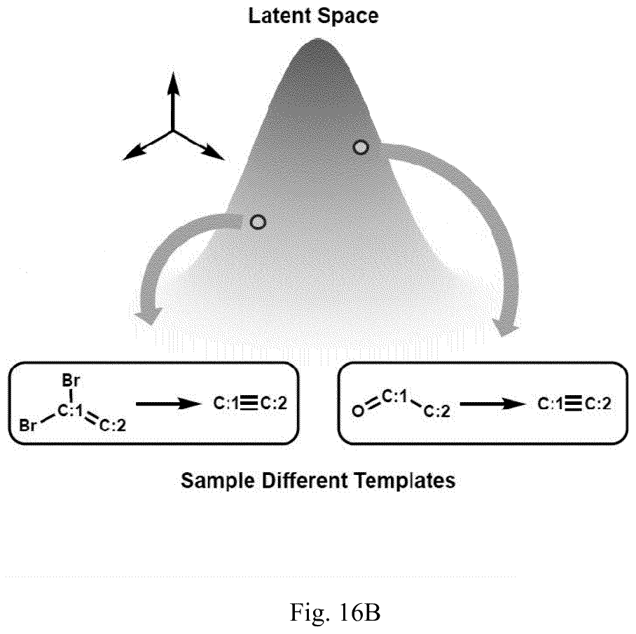

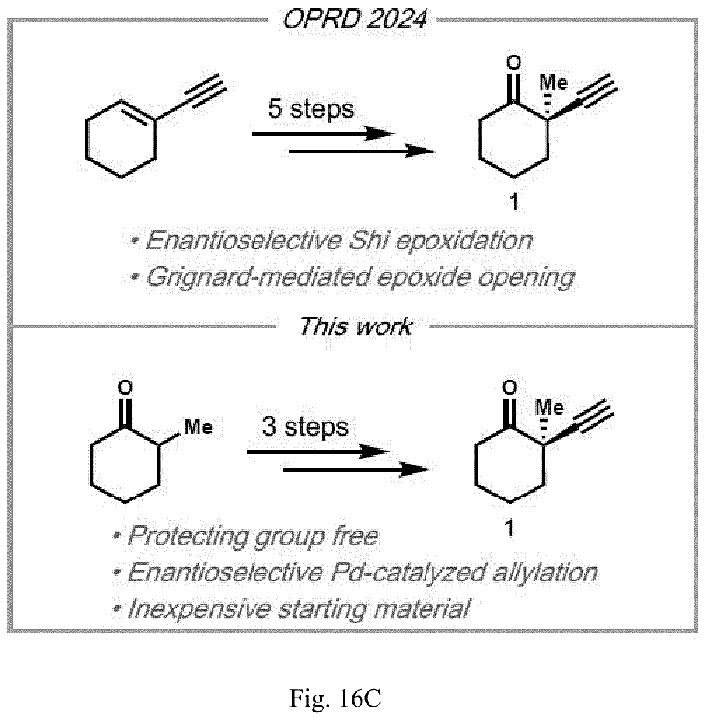

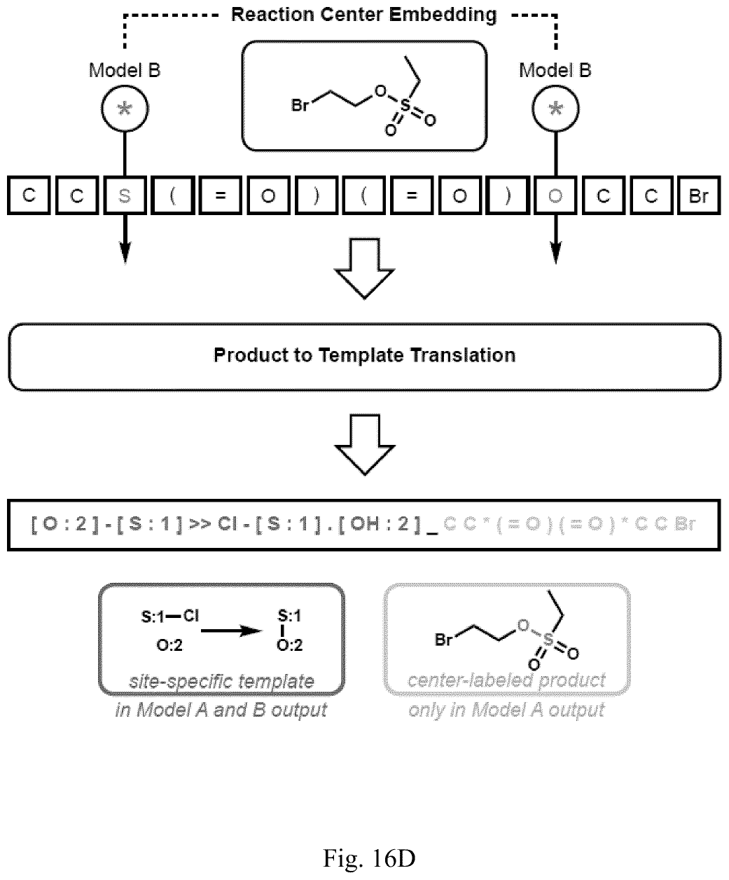

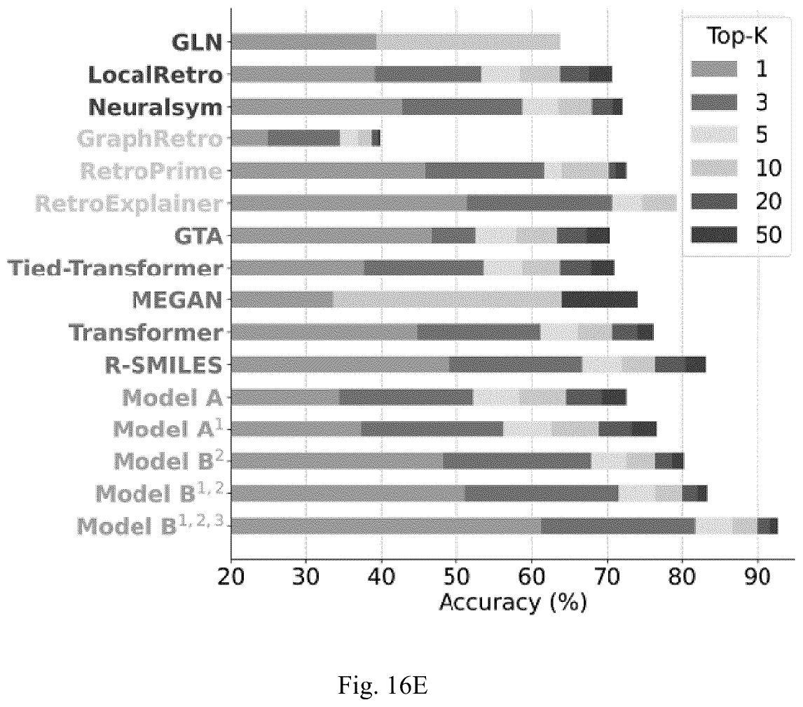

FIGS. 16A through 16C show common machine learning methods for retrosynthesis as well as an exemplary method disclosed herein. FIG. 16A is a diagram showing that reactants and templates can be selected and generated based on a target compound using different machine learning models. In one example, template generation is used in the disclosed method. FIG. 16B is a diagram showing that latent space is incorporated in one of the models in the disclosed method according to aspects of the present invention. In some embodiment, the method comprises sampling in latent space in order to give different reaction templates. FIG. 16C shows the results of the disclosed method compared to a previous method, displaying a reduction in synthesis steps for a key intermediate for active pharmaceutical ingredients (API).

Other aspects of the invention relate to a sampling generative model (sampling model) for template generation that applies to a target product. In some embodiments, the disclosed sampling model has a latent space, enabling the generation, interpolation, and distance measurement of various templates (FIG. 16B). Aspects of the present invention also relate to deterministic models that take target compounds as input and generate templates. For example, but without limitation, in some embodiments the encoder of the model can incorporate positional embedding for reaction centers, enabling users to specify specific reacting sites during prediction where the results are benchmarked on the USPTO-FULL dataset.

The disclosed sampling model is partially based on the conditional kernel-elastic autoencoder (CKAE) also disclosed herein, which is the first of its kind in the field of retrosynthesis. This model conditions on corresponding products during training, allowing interpolating and extrapolating capabilities of reaction templates in the latent space to generate templates during the sampling process. The latent space also provides a measure of distances between reaction templates, allowing means to identify the closest reaction reference within the dataset or determine the similarity between two reactions.

The disclosed template generation method introduces a novel design and feature where the templates, which are referred to herein as site-specific templates (SSTs), exploit just the reaction centers of the involved molecules. This results in a concise and informative set of templates different from the templates available in the RDChiral repository [Connor W. Coley et al., J. Chem. Inf. Model., June 2019]. Additionally, SSTs and target compounds with reaction centers labeled (center-labeled product, CLP) are simultaneously encoded/decoded, allowing the model's attention mechanism to incorporate reaction centers defined by atoms in the molecule context. Integrating these features into the template generation process ensures the relevance and practicality of the generated templates. In addition, to resolve the common problem of having new unidentified reactions, CKAE's latent space is used to establish distance measurement which allows the referencing of reactions within the training set.

With SSTs and generation methods in place, the disclosed approach was validated through the practical application of synthesis. Compound lb-7 was reported by Boehringer Ingelheim [Jason ABBOTT et al., U.S. Patent 2023/0212164 A1, 2023] along with a library of analogs, as a potent Ba/F3 KRASG12C inhibitor, and potential anti-cancer agent. The synthetic route for lb-7 has two key intermediates (FIG. 16C), a thiophene derivative and its precursor compound 1. A cyclohexanone with quaternary chiral center in α-position containing alkyne moiety is considered a synthetic challenge. A machine learning model coupled with human intuition was used to determine the most step-efficient way to synthesize compound 1, thereby reducing the number of steps from 5 to 3 compared to previous work [Jason ABBOTT et al., U.S. Patent 2023/0212164 A1, 2023]. The disclosed experimental examples provide insights into the practicality and reliability of retrosynthesis predictions, reinforcing the models' robustness and their underlying promise to address a wide spectrum of retrosynthesis problems.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the system and method of the present invention. The following working examples therefore, specifically point out the exemplary embodiments of the present invention, and are not to be construed as limiting in any way the remainder of the disclosure.

### Example 1: Kernel-Elastic Autoencoder

Disclosed herein is an innovative self-supervised generative approach called Kernel-Elastic Autoencoder (KAE), which enhances the performance of traditional Variational Autoencoder by designing both modified maximum mean discrepancy and weighted reconstruction loss functions. KAE addresses the long-standing challenge of achieving superior generation and reconstruction performances at the same time. The disclosed Transformer-based model encodes molecules as SMILES strings and demonstrates outstanding results in molecular generation tasks. The model achieves remarkable diversity in molecule generation while maintaining near-perfect reconstructions (over 99%) on the testing dataset, surpassing previous molecule-generating models. The model's functionality was extended by enabling conditional generation and implementing beam search in the decoding phase for improved molecule candidate search in constraint optimization tasks, resulting in a significant 28% improvement over the baseline. Furthermore, the disclosed model shows promise in molecular docking tasks, enriching the dataset with higher-scoring candidates and outperforming training set molecules according to both AutoDock Vina and Glide. The disclosed work represents a substantial contribution to generative models for molecular design and optimization, demonstrating the strength and potential of the disclosed approach.

With the idea of generation, a variety of applications for drug discovery and Chemistry are made available [Maziarka et al., Journal of Cheminformatics, 2020; Moret et al., Nature Machine Intelligence, 2020; Skalic et al., Journal of chemical information and modeling, 2019; Wang et al., Nature Machine Intelligence, 2021]. Variational Autoencoder (VAE) [Diederik P Kingma and Max Welling. Auto-encoding variational bayes. arXiv preprint arXiv:1312.6114, 2013] stands out as a pioneer of generative models. Its versatility in molecule generation is explored by many works formats such as character [Gómez-Bombarelli et al., ACS central science, 2018], grammar [Kusner et al., In International conference on machine learning, pages 1945-1954. PMLR, 2017], and graph-based [Jin et al., In International conference on machine learning, pages 2323-2332. PMLR, 2018] VAEs.

VAE differs from Autoencoder (AE) [Dana H Ballard. Modular learning in neural networks. In Aaai, volume 647, pages 279-284, 1987] in that it achieves generation purpose by modeling data as probabilistic distributions. Whereas the goal of AE is to efficiently embed data into a low-dimensional space. However, the latent space of the AE often has regions that do not correspond to any encoded data and therefore sampling around encoded latent representations is not feasible. The loss function for sequence-to-sequence style VAE seeks to reconstruct cross-entropy term as implemented in AE, while treating each latent vector as a distribution. Then by enforcing all latent vectors to prior distribution such as a Gaussian, decoding latent vectors sampled from this distribution gives results that resemble training data. Not limited by generation, VAEs' potentials are explored widely in molecule property prediction and optimizations which are important steps in computational drug discovery.

The VAE performance for molecule generation is measured in terms of novelty (N), uniqueness(U), validity(V), and reconstruction (R). Though all VAE models aim to achieve the highest metrics, they are constrained by a trade-off between the NUV and reconstruction. A model that reconstructs well might not be able to achieve high NUV and vice versa. Whichever gets closer to the optimum, the other will suffer. For example, in the case of molecule generation, if a VAE model is capable of reconstructing the input unambiguously, inferencing on latent vectors that the decoder has not seen before would be unpractical, being less likely to produce valid outputs. This trade-off leads to additional concerns, especially since these models lack the ability to perform precise optimization around a target molecule [Madhawa et al., arXiv preprint arXiv:1905.11600, 2019]. Being able to reconstruct is important because it ensures success in interpolating between molecules in latent representation as well as sampling close molecular scaffolds for a given target.

Graph-based VAE methods take the lead in chemical validity [Jin et al., In International conference on machine learning, pages 2323-2332. PMLR, 2018; Jin et al, In International conference on machine learning, pages 4839-4848. PMLR, 2020]. This is because the molecules are represented in graphs of motifs and these motifs explicitly enforce grammar rules onto the molecules, so the generated molecules are all valid. However, this is not the case without checking for grammar; during testing, if certain motifs do not exist in the training dataset, the model would not be able to reconstruct or sample similar molecules.

Flow-based generative models, on the other hand, excel in the reconstruction by memorizing the training dataset [Zang et al., In Proceedings of the 26th ACM SIGKDD International Conference on Knowledge Discovery & Data Mining, 2020; Luo et al., In International Conference on Machine Learning, pages 7192-7203. PMLR, 2021]. The models consist of invertible maps from input data to latent space with the same dimension so the model can map latent vectors back to the input molecules exactly. Nevertheless, same-dimensional latent representations are criticized for not being able to capture features that are important and tend to overfit. Out-of-distribution problems could arise during the sampling process and the reconstruction on the testing dataset remains a concern [Nalisnick et al., arXiv preprint arXiv:1810.09136, 2018].

In this work, a new architecture is designed and is referred to as Kernel-Elastic Autoencoder (KAE) which greatly reduces the aforementioned problems. Disclosed is a new loss function that combines the benefits of both the AE and VAE objectives. The framework captures both behaviors. KAE achieves state-of-the-art performance in the generation task without any checks of molecule grammar or chemical rules while reaching near 100% reconstruction on a 24k-molecule test set. In KAE, the Kullback-Leibler (KL) divergence loss [James M Joyce. Kullback-leibler divergence. In International encyclopedia of statistical science, pages 720-722. Springer, 2011] normally used in VAE is replaced with an MMD-inspired loss function, modified MMD, to shape the latent space. By incorporating the kernel used for the MMD-inspired term and allowing weighing between AE and VAE objectives, it was believed this is widely applicable as it presents a new way to obtain higher performance implementable for all other VAE and AE-based architectures on datasets outside of molecule generations.

In the disclosed work, the performance from a fully transformer-based [Vaswani et al., Advances in neural information processing systems, 30, 2017] architecture was leveraged. Under the same architecture, it was compared and shown that the modification of either term from the original VAE loss function leads to better performances. With both modifications, KAE stands out from other string and graphical-based models in a variety of performance measures (N, U, V, R and optimization tasks).

The KAE architecture and the KAE loss were presented that optimize model performance by comparing it to the KL-based loss function. In addition, it was demonstrated that the result, in combination with beam search techniques as adopted by [Moret et al., Angewandte Chemie International Edition, 2021; Tetko et al., Nature communications, 2020], rivals the results produced by graphical models which are known to produce the highest validity SMILES after grammar checks. It was proposed to use beam search for the generation process and demonstrate that this approach can be used to increase sample diversity. It was further shown that different interpretations for the same latent vectors can be derived exclusively with beam search.

The inefficiencies of the existing models were identified in constraint optimization tasks with less accurate reconstructions and the performance from state-of-the-art results were improved [Ryan J Richards and Austen M Groener. Conditional β-vae for de novo molecular generation. arXiv preprint arXiv:2205.01592, 2022] by over 28%, while also exceeding the metric arising from searching candidates in the training dataset.

To demonstrate the applicability of the model, conditioned searches for docking candidates were performed with training dataset obtained from [Bengio et al., arXiv preprint arXiv:2111.09266, 2021]. The conclusion of having better candidates from the baseline is separately confirmed by both Autodock Vina and Glide.

### Model Architecture

The problem was formatted as a translation task that takes the source language, which is encoded and compressed as latent vectors, to the decoded target language. Leveraging the model's ability for semantic interpretation of the SMILES grammar rules, the bulk architecture of Transformers were used [Vaswani et al., Advances in neural information processing systems, 30, 2017] that utilized both self and encoder-decoder attentions. FIG. 1 shows the KAE model's architecture with the option for adding conditions. The disclosed model is composed of a Transformer encoder, compression layer, expansion layer, and Transformer decoder. Attention operations are performed with four heads. For each input, with specified padding tokens, both the source and target masks are made to prevent the model from attending to paddings during training. SMILES tokens are passed through the encoder and decoder embedding layers that transform each token into an E-dimensional vector where E is the embedding size and is equal to 128 for all implementations discussed in this work. These vectors are added to the encoder and decoder specific E-dimensional positional embeddings. In Conditional KAE (CKAE), additional embeddings are used just for attached condition(s) such as different molecule properties. CKAE's condition-scaled embeddings are concatenated to both the input of the encoder and the latent representation along the sequence length dimension, allowing the model to generate molecules by interpolating and extrapolating with asked properties as conditions.

The encoder input is processed by the Transformer encoder followed by a compression in the sequence length dimension. This latent vector of 10×E dimensions (a 10-dimensional compressed sequence length by E embedding size) is injected with noise during training. In the case of CKAE, it is concatenated with the property-scaled embedding vector condition. This processed latent vector is mapped back to M×E dimensions by the expansion layer where M is the maximum sequence length in the relevant dataset. This vector is, treated as the final encoder output, fed into the decoder without supplying encoder masks. Each decoder layer attends to the encoder outputs through the encoder-decoder muti-head attention operations. Outputs are contracted by a linear layer along the embedding dimension to produce a T-dimensional vector per token. This T-dimensional character is then softmaxed, and interpreted as a probability distribution (P) for each possible character (c).

### Training Datasets

On the ZINC250K dataset. The model is trained on 225k (90%) of the entries. Within the other split, 1k molecules are used for validation and 24k are used for testing. Depending on the purpose, the training is either using (molecular properties) or having zero conditions.

For the dataset with 300k docking candidates from [Bengio et al., arXiv preprint arXiv:2111.09266, 2021], all entries are used for training.

### KAE Loss Function

The disclosed loss is different from that for VAE [Diederik P Kingma and Max Welling. Auto-encoding variational bayes. arXiv preprint arXiv:1312.6114, 2013] as both the reconstruction objective and distribution measurement are modified.

The original VAE loss is framed with a loss

$${ℒ}_{VAE} = {ℒ}_{CEL} + {ℒ}_{𝒦ℒ} \qquad Equation 1$$

where the first term [symbol] _{CEL} denotes the cross-entropy loss (CEL, reconstruction objective) and the second term is the Kullback Leibler divergence (KL-divergence, distribution measurement). The summations over s and c are for sequence length and the number of tokens in the decoding dictionary. Y is equal to one if the token belongs to the class c at position s and is zero otherwise.

The objective was reformulated from [symbol] _{CEL} to a weighted cross-entropy loss (WCEL). The distribution-related KL loss is changed to a Maximum-Mean-Discrepancy-inspired (MMD) term which is referred to as modified-MMD (m-MMD).

During training, both the decoder output and the latent vector are retrieved for loss calculations. The decoder outputs are penalized with the teacher forcing method [Lamb et al., Advances in neural information processing systems, 2016]. The latent vectors, however, are penalized by their difference from 1000 randomly sampled Gaussian vectors (\vec{Gᵢ}) using kernel-based metrics. The WCEL, denoted as for [symbol] _{w}, sequence is expressed as

$${ℒ}_{WCEL} ( λ , δ ) = \frac{1}{λ + δ + 1} [ {\sum }_{s} {\sum }_{c} {Y}_{s , c} log ( {P}_{s , c} ) + ( λ + δ ) {\sum }_{s} {\sum }_{c} {Y}_{s , c} log ( {P}_{s , c}^{*} ) ] \qquad Equation 2$$

The P_{s,c}* is the model estimated probability for the pair of s and c for the latent representation without added training noises. λ is related to the m-MMD term and δ is another hyper-parameter that controls the significance of the second term or the AE behavior.

The weighing ((λ+δ) ∈[0, ∞)) of the second term allows the learning objective to stay between VAE and AE objectives. With special cases of the two ends of the bound, the objective becomes VAE or AE-like. λ is included in the second term because as λ becomes larger, the model restricts latent vectors closer together as penalized by m-MMD loss. This effect increases the probability of sampling valid latent vectors but decreases distinctions between vectors. This effect from λ is detailed as described herein.

λ is a scalable parameter that adjusts the weight of this term relative to the CEL. [symbol] is a radial basis function kernel with

$$𝒦 ( \vec{x} , {\vec{G}}_{i} ) = exp ( \frac{- \frac{1}{D} {\sum }_{d = 0}^{D} {( {G}_{i , d} - {x}_{d} )}^{2}}{2 {σ}^{2}} ) \qquad Equation 3$$

where D is the size of the latent dimension and is equal to 10 times the embedding dimension (10×E). The value of 2σ²=0.64 was empirically chosen (model performance with different sigma values is described further herein).

Overall, the disclosed loss is expressed as:

$$ℒ ( λ , δ ) = {ℒ}_{WCEL} ( λ , δ ) + m - MMD ( λ ) . \qquad Equation 4$$

During training, a noise ∈∈ [symbol] from a gaussian with a mean of zero and standard deviation of one is added to the latent vector before the latent vector is sent to the decoder.

m-MMD and s-MMD

The original MMD loss between \vec{x} and \vec{j} is calculated as

$${‖ \vec{{μ}_{x}} - \vec{{μ}_{y}} ‖}_{ℱ}^{2}$$

where \vec{μₓ} and \vec{u_{y}} represent the first moments of ϕ(\vec{x}) and ϕ(\vec{y}) and ϕ is a map to space [symbol] . The MMD loss can be expanded as

$${\vec{{μ}_{x}}}^{T} \vec{{μ}_{x}} + {\vec{{μ}_{y}}}^{T} \vec{{μ}_{y}} - {\vec{{μ}_{x}}}^{T} \vec{{μ}_{y}} - {\vec{{μ}_{y}}}^{T} \vec{{μ}_{x}} . \qquad Equation 5$$

A function [symbol] is defined as the kernel function (Equation 3 such that [symbol] (\vec{x}, \vec{y})=\vec{ϕ(x)},^{T}, \vec{ϕ(y)} is the inner product between \vec{x} and \vec{y} in space [symbol] through the transformation ϕ.

The first moment of \vec{x} is calculated as

$$\vec{{μ}_{x}} = \frac{1}{{N}_{x}} {{\sum }_{i}}^{{N}_{x}} ϕ ( \vec{{x}_{i}} ) .$$

This allows inner products between \vec{μ_{α}} and \vec{μ_{β}} to be written as

$${\vec{{μ}_{α}}}^{T} \vec{{μ}_{β}} = \frac{1}{{N}_{α} {N}_{β}} {{\sum }_{i}}^{{N}_{α}} {{\sum }_{j}}^{{N}_{β}} {ϕ ( \vec{{α}_{i}} )}^{T} ϕ ( \vec{{β}_{j}} ) . \qquad Equation 6$$

In the kernel representation, Equation 6 is written as:

$${\vec{{μ}_{α}}}^{T} \vec{{μ}_{β}} = \frac{1}{{N}_{α} {N}_{β}} {{\sum }_{i}}^{{N}_{α}} {{\sum }_{j}}^{{N}_{β}} 𝒦 ( \vec{{α}_{i}} , \vec{{β}_{j}} ) \qquad Equation 7$$

When Equation 7 is implemented, since all j are sampled from the target Gaussian distribution, the \vec{μ_{y}},^{T} \vec{μ_{y}} term is not involved in the computational graph during gradient descent. And, since the kernel function is symmetric, the standard-MMD (s-MMD) loss is reduced to

$$s - MMD ( λ ) = λ [ \frac{1}{{N}_{x}^{2}} {{\sum }_{i}}^{{N}_{x}} {{\sum }_{j}}^{{N}_{x}} 𝒦 ( \vec{{x}_{i}} , \vec{{x}_{j}} ) - \frac{2}{{N}_{x} {N}_{y}} {{\sum }_{{i}^{′}}}^{{N}_{x}} {{\sum }_{{j}^{′}}}^{{N}_{y}} 𝒦 ( \vec{{x}_{{i}^{′}}} , \vec{{y}_{{j}^{′}}} ) ] \qquad Equation 8$$

In the case of a zero-minimum inner product, the minimum of the first term is achieved at \vec{μₓ} being zero. Minimizing the first term promotes all ϕ(\vec{xᵢ}) to spread out in the space [symbol] while the second term encourages ϕ(\vec{x}) to be close to the distribution of ϕ(\vec{y}). The m-MMD loss can also be seen as

$$m - MMD ( λ ) = λ [ 1 - \frac{1}{{N}_{x} {N}_{y}} {{\sum }_{i}}^{{N}_{x}} {{\sum }_{j}}^{{N}_{y}} 𝒦 ( \vec{{x}_{i}} , \vec{{y}_{j}} ) ] \qquad Equation 9$$

### Decoding Methods

To generate a new molecule, a D-dimensional Gaussian distribution was first sampled to obtain vector \vec{v} where \vec{v}∈ [symbol] ^{10×ε}. In CKAE this vector is then concatenated with the condition C that is multiplied by its corresponding embedding vector. The final vector is decompressed by the expansion layer to \vec{L}∈ [symbol] . The Decoder translates a sequence of SMILES string, character-by-character, with encoder-decoder attention applied to this vector.

When decoding a single sequence, the “<SOS>” token is first fed to the decoder. The decoder then performs multi-headed attention from its input to the encoder output \vec{L} and produces a probability distribution over T possible tokens for each input. At this step, the common method is to continue the prediction with the token having the maximum probability by concatenating the token to the next-round input sequence, and repeating the procedure again to obtain the next most probable token until the “<EOS>” token is produced or the maximum sequence length is reached. Instead of keeping only the most probable token, with beam size as a hyper-parameter, beam searches were performed to derive more possible interpretations from the same vector \vec{L}. With a beam size of B, B<=T, there are maximum B outputs generated from one decoding procedure.

The beam search algorithm records the probability of each step for each of the B sequences. For the first step in beam search, the top B most probable tokens are selected. For the following steps, the model will decode from B input sequences at the same time. Since each of the B sequences have T number of possible outcomes for the next token, the total number of possible next-step sequences is B×T. These sequences are then ranked based on the sum of their probabilities for all S characters. In beam search, the probability of a sequence of tokens indexed from s, s−1, s−2 . . . to 0 can be represented as

$$P ( s , s - 1 , s - 2 , … 0 ) = P ( s \vert s - 1 , s - 2 , … 0 ) \times P ( s - 1 , s - 2 , … 0 ) \qquad Equation 10$$

This expression can then be treated as the product of the individual probabilities where

$$P ( s , s - 1 , s - 2 , … 0 ) = P ( s \vert s - 1 , s - 2 , … 0 ) \times P ( s - 1 \vert s - 2 , s - 3 … 0 ) \times … P ( 0 ) \qquad Equation 11$$

However, since every term is less than one, when calculating long sequences, Equation 11 produces intractable small numbers. Therefore, the sum of the log probabilities is calculated instead. For the B×T sequences with the same sequence length S the probability of the ith sequence at every position s is denoted as P_{i,s}; Without counting the probabilities of padding tokens, the sum of log probabilities, Pᵢ for the i'th sequence is calculated as:

$${P}_{i} = \frac{1}{\sqrt{{N}_{i}}} {\sum }_{s \neq pad}^{S} Log ( {P}_{i , s} ) \qquad Equation 12$$

where Nᵢ is the number of non-padding tokens in sequence i.

To encourage the variety of decoding, sequence lengths are into account while calculating Pᵢ. The

$$\frac{1}{\sqrt{{N}_{i}}}$$

term reduces the preference for shorter sequences over longer sequences as longer sequence tends to have smaller sums of log probabilities.

The top B most probable tokens are selected and used as the inputs for the next iteration until the maximum sequence length M is reached or all top B candidates have produced the “<EOS>” indicating the end of decoding.

The experimental results are now described herein.

Generation performance is measured in terms of the following three metrics: Novelty (N), Uniqueness (U), and Validity (V). A valid molecule is novel if it does not belong to the training dataset, and is unique if it is not already generated. Valid means the SMILES representation of a molecule is both syntactically correct and has valid chemical semantics as checked by RDKit. Reconstruction (R) is considered successful if the decoder outputs characters that exactly match those in the input SMILES.

### Comparisons Between Different Distribution Constraint Loss Terms

The m-MMD was chosen over s-MMD, and over the traditional KL term as in the case of a traditional VAE. Performances of the models with these specifications are compared during their 200 epochs of training. For all tests in this section, the λ and δ terms in all the loss functions are set to 1 and −1 (when λ=−δ, [symbol] is reduced to [symbol] ). In addition, the effect of latent noise on MMD-based losses was shown. m-MMD loss injected with Gaussian noise in the latent space achieves the best result of all the options.

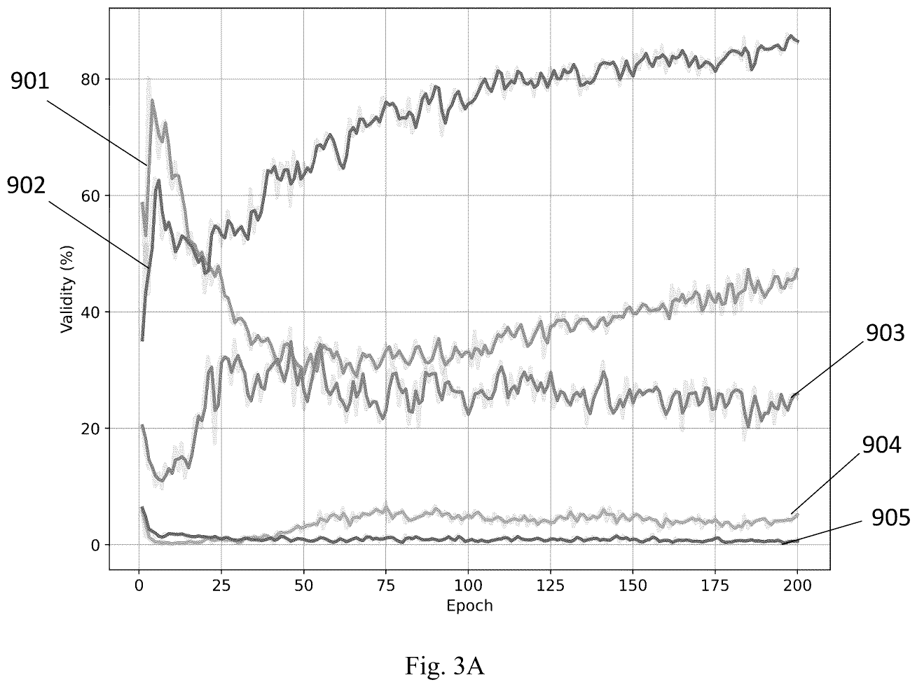

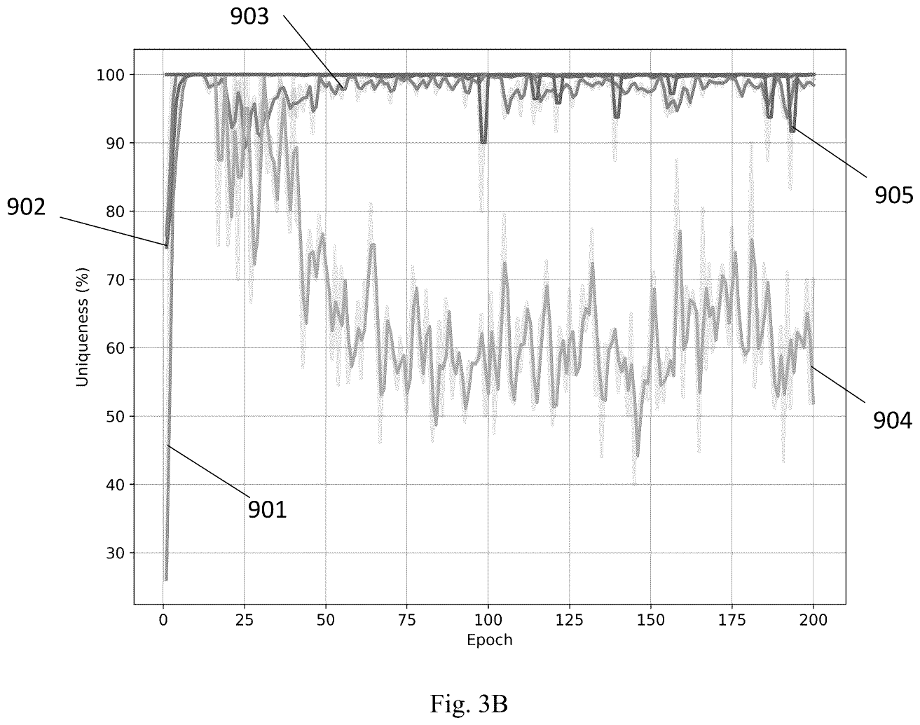

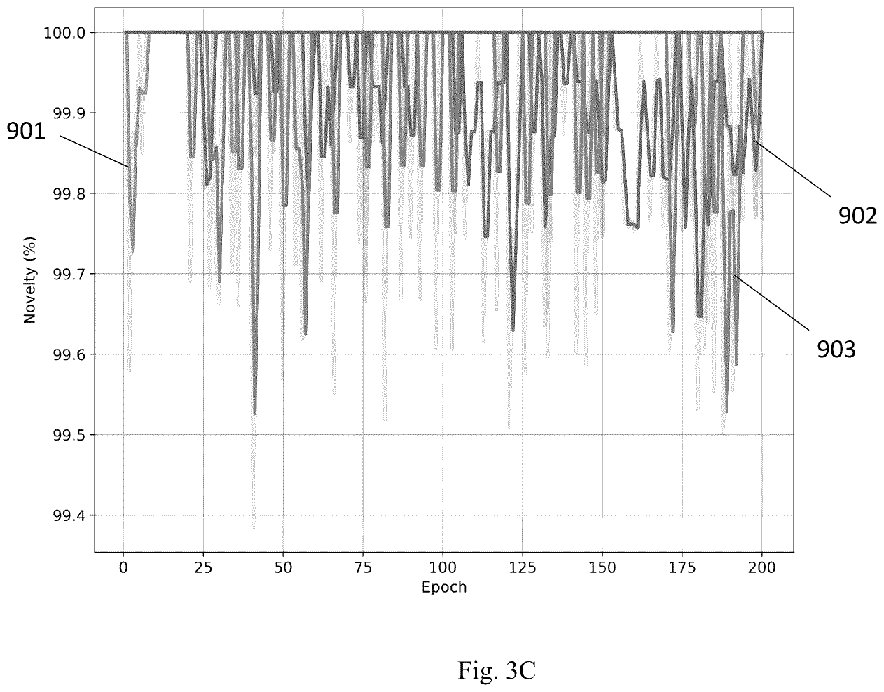

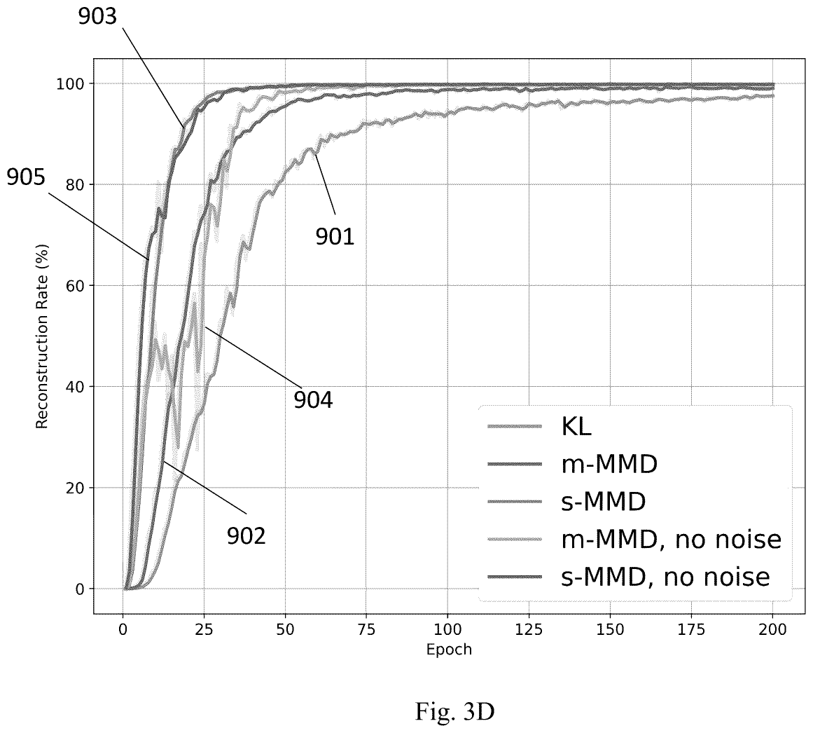

The performances of models trained with three different loss functions are compared in FIG. 3. FIGS. 3A, 3B, 3C, and 3D show a performance comparison of the models trained with modified MMD (m-MMD) loss, standard MMD (s-MMD) loss, and Kullback-Leibler (KL) divergence loss: 901 KL; 902 m-MMD; 903 s-MMD; 904 m-MMD, no noise; 905 s-MMMD, no noise. The addition of noise tests only applies to MMD-related models. The loss functions are [symbol] adding a second term chosen from m−MMD(λ), s−MMD(λ), and KL divergence respectively. The ratios between [symbol] and the second terms are 1:1 for all cases of λ=1 throughout the training process.

1k latent vectors are sampled to evaluate the models' validity, uniqueness, and novelty at each epoch during training. Reconstruction rates are calculated based on 1k molecules from the validation set. It is observed that in cases where noise is not present, both the s-MMD and m-MMD models have near-zero validity values which made their performance significantly worse than their counterparts with noise added during training.

The models trained with KL loss noised s-MMD, and noised m-MMD have dramatic differences in validity compared to their uniqueness and novelty metrics. Their reconstruction rates converge and the model with KL loss is the slowest of all. In conclusion, the performance, as measured in NUV values at the 200-epoch, of m-MMD is better than the s-MMD. Adding noise helps sampling valid molecules; m-MMD with added noise is better than training with KL divergence loss for the disclosed architecture.

### Effects of the λ Parameter

The reason for increasing λ is similar to that of increasing β in the case for β-VAE [Higgins et al., In International conference on learning representations, 2017]. Both λ in KAE and β in β-VAE encourage the model to learn more efficient latent representations and construct smoother latent space. However, since KAE has different architecture and loss objectives from VAE, the aforementioned regularization do not lead to the same result in terms of NUVR as observed in the case when both λ and β are set to one for KAE and VAE.

For the best model using m-MMD in FIG. 3, all validities are lower than 90%. This is improved by increasing the λ value for the m-MMD term as shown in Table 1. The models in Table 1 were first trained with λ=1 for 85 epochs then with higher values for an additional 1 epoch. δ values were set to −λ throughout the training process to exclude any effects from WCEL in the comparison.

The higher the λ the tighter the model will place the latent vectors together according to the m-MMD loss. This is reflected by the increase in the probability of sampling valid molecules when the latent vectors are drawn from the same distribution. However, as all latent vectors are becoming closer, it becomes harder for the decoder to differentiate them, which is reflected by the decrease in reconstruction. And the decreased uniqueness and novelty with increased λ are due to that the decoder more often identifies different molecules with overlapping latent representations as the same ones.

The overall effects of λ are shown by the product of the N U and V (NUV) as well as the one including reconstruction (NUVR).

Table 1 shows the trend of NUV and NUVR as λ is adjusted. It is observed that validity peaks with larger λ and the model trained with λ=24.5 has the highest NUV. However, the reconstruction rate decreases significantly with increasing λ values.

Table 1 shows the result of sampling 1k latent vectors after training the model from the same checkpoint (85 epochs) with the loss function being [symbol] (λ=1, δ=−1) but then followed by an additional epoch with different λ values (loss functions are then [symbol] (λ=λ, δ=−λ)).

TABLE 1
Model performance with varying λ

| λ | Validity | Novelty | Uniqueness | NUV | Reconstruction | NUVR |
|---|---|---|---|---|---|---|
| 1.0 | 0.782 | 1.000 | 0.995 | 0.778 | 0.988 | 0.769 |
| 2.0 | 0.802 | 1.000 | 1.000 | 0.802 | 0.978 | 0.784 |
| 5.0 | 0.849 | 1.000 | 1.000 | 0.849 | 0.933 | 0.792 |
| 10.0 | 0.847 | 0.999 | 0.999 | 0.845 | 0.792 | 0.669 |
| 15.0 | 0.913 | 0.998 | 1.000 | 0.911 | 0.527 | 0.480 |
| 20.0 | 0.929 | 1.000 | 1.000 | 0.929 | 0.246 | 0.229 |
| 24.5 | 0.961 | 0.999 | 0.998 | 0.958 | 0.060 | 0.057 |
| 25.0 | 0.940 | 0.998 | 1.000 | 0.938 | 0.043 | 0.040 |
| 25.5 | 0.943 | 1.000 | 1.000 | 0.943 | 0.039 | 0.037 |
| 26.0 | 0.965 | 0.998 | 0.999 | 0.962 | 0.029 | 0.028 |
| 27.5 | 0.962 | 0.996 | 0.999 | 0.957 | 0.010 | 0.010 |
| 30.0 | 0.970 | 1.000 | 0.987 | 0.957 | 0.000 | 0.000 |

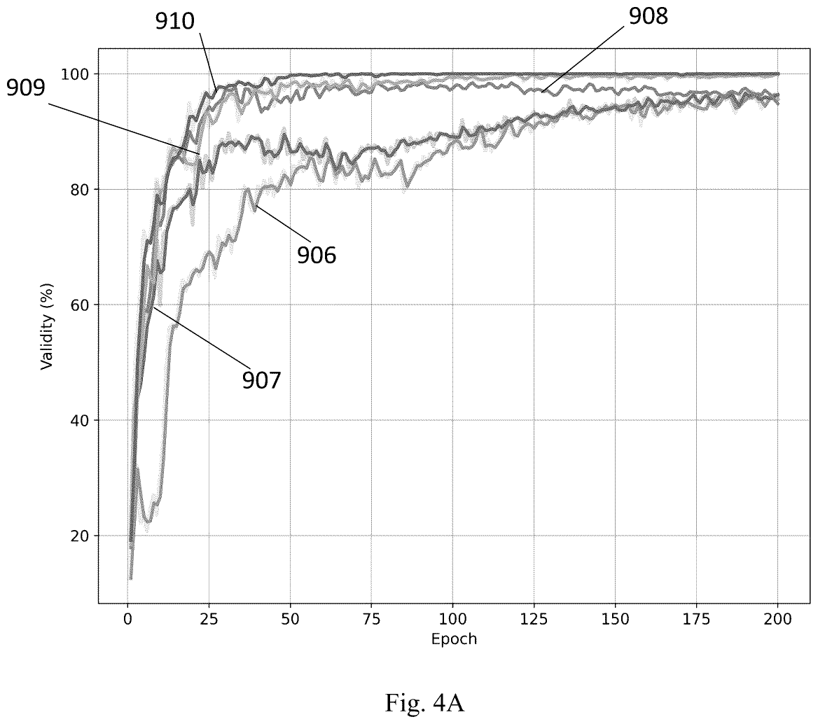

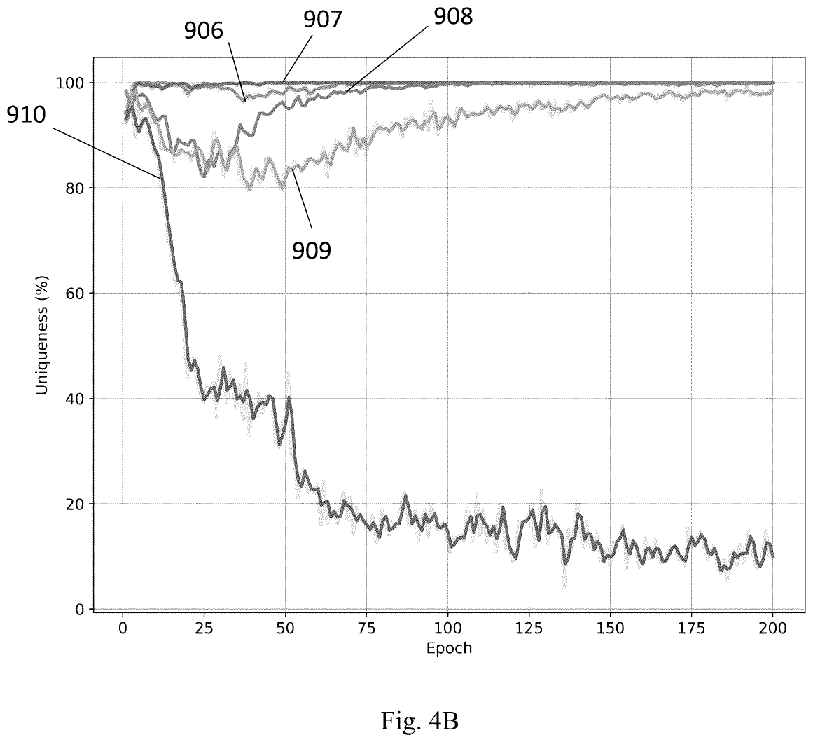

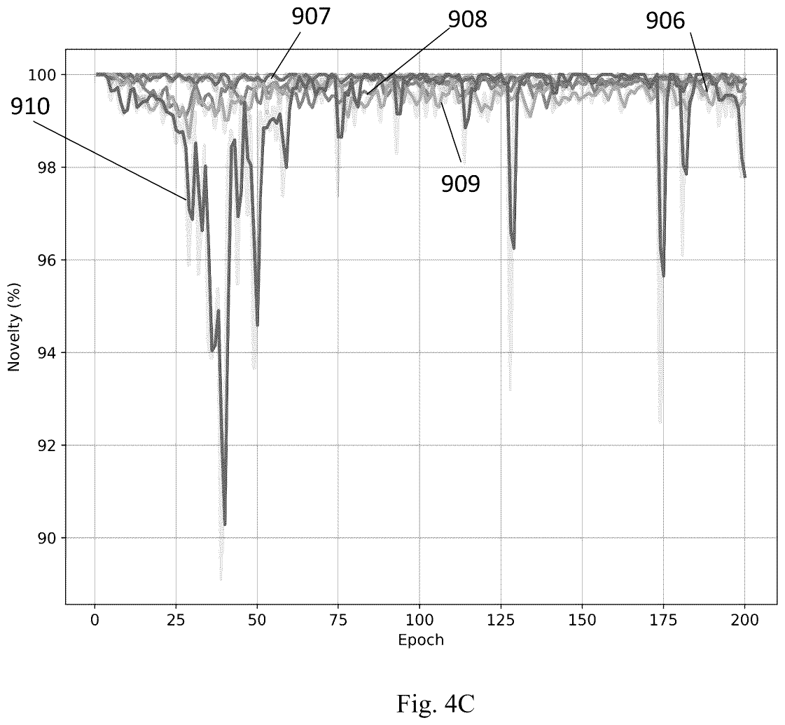

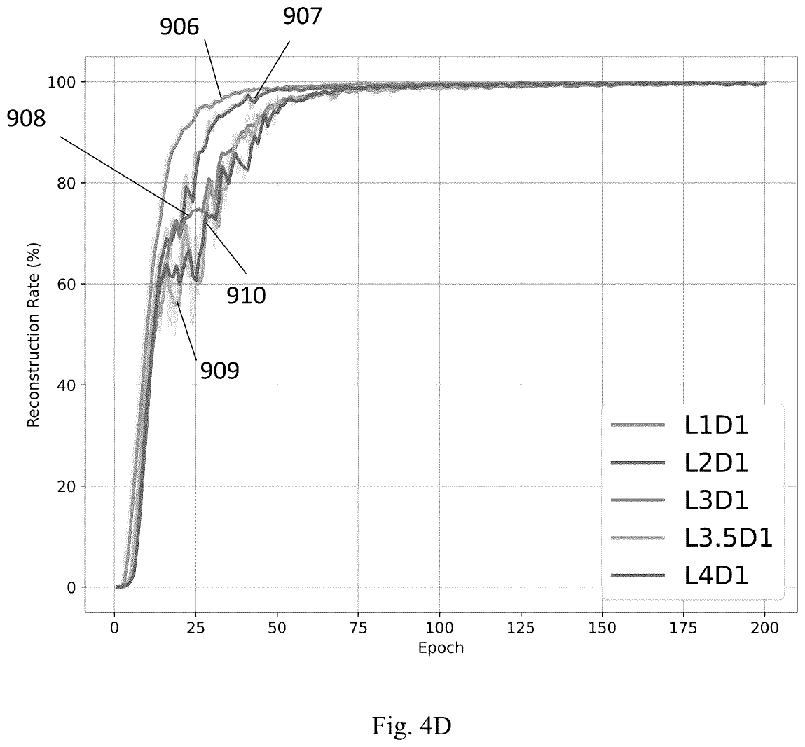

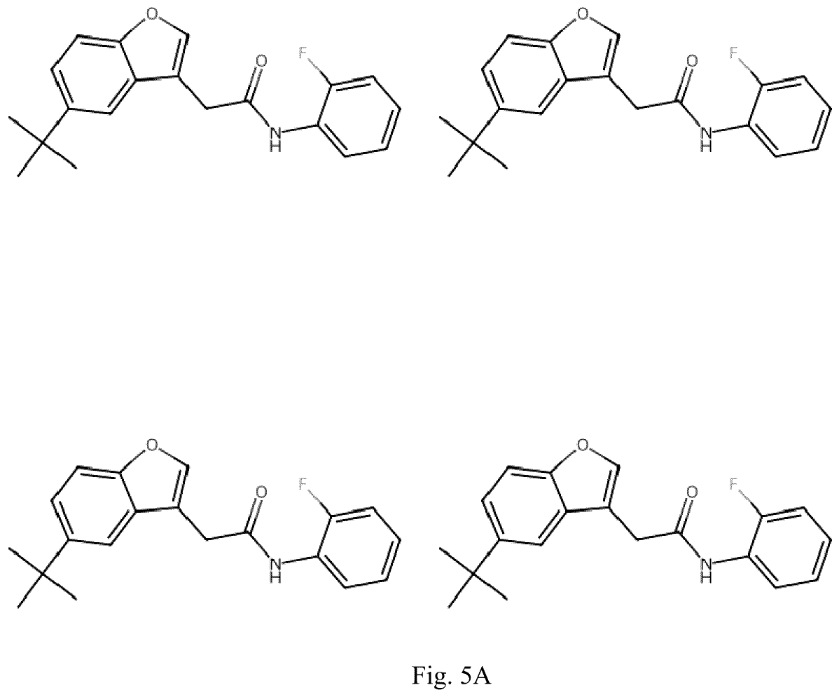

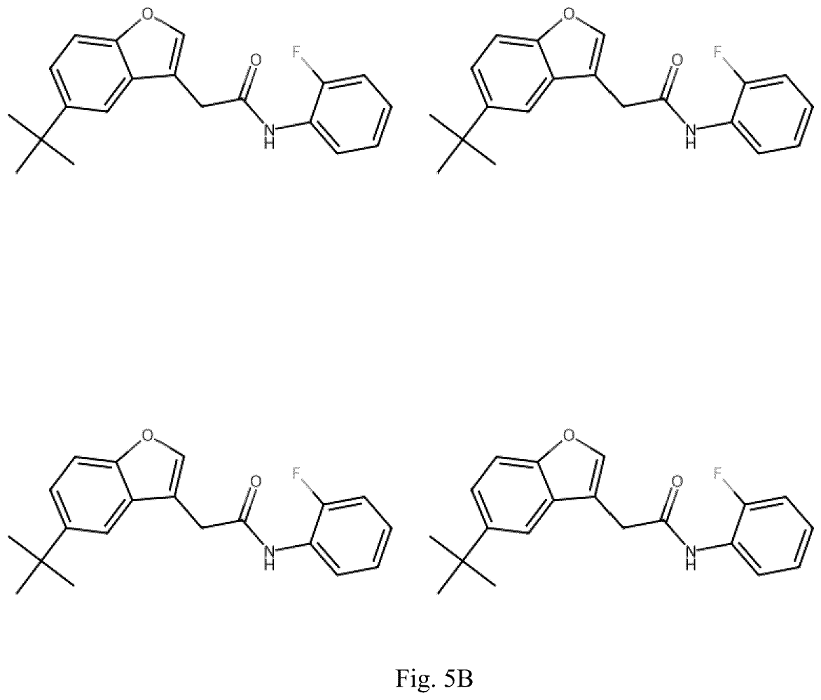

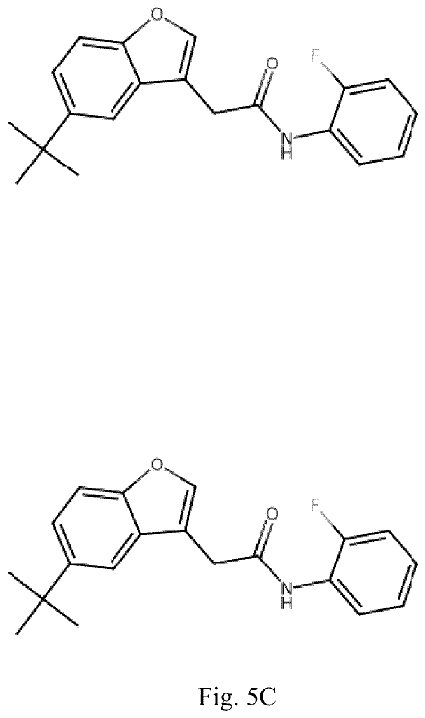

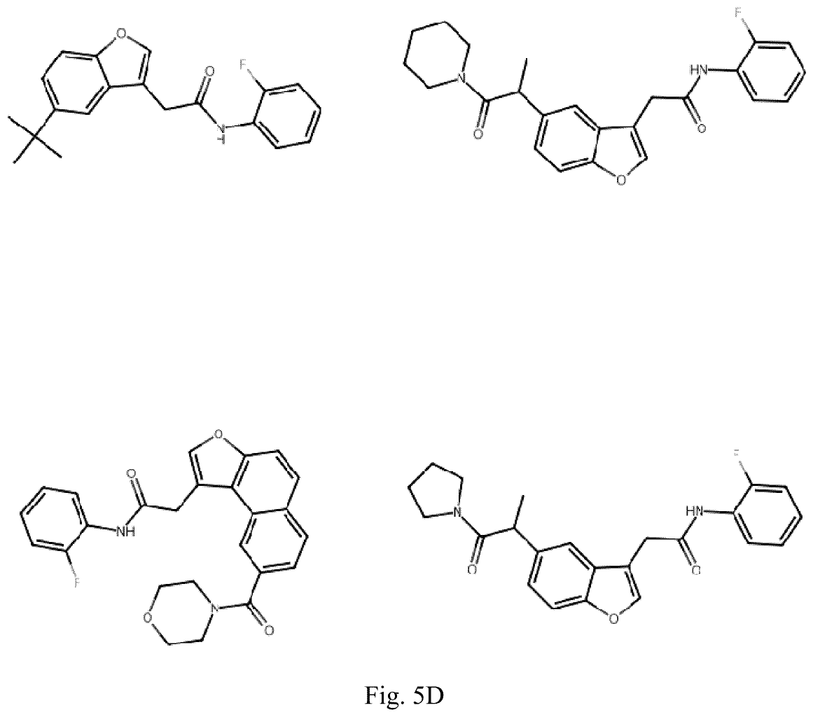

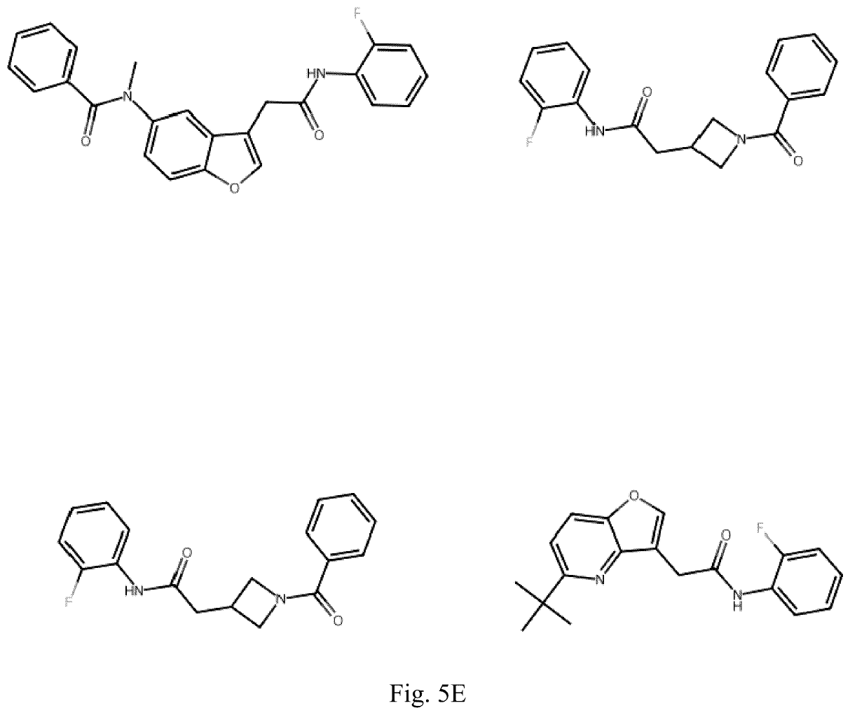

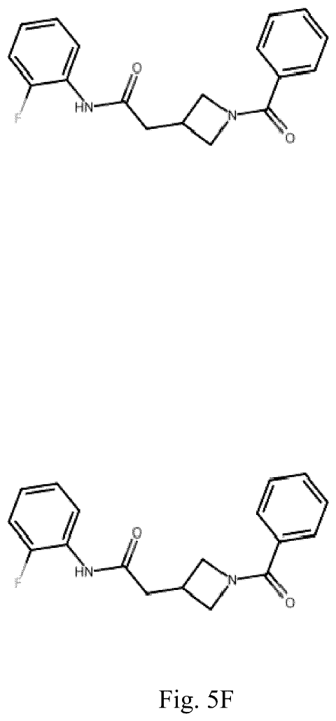

A solution was sought that can increase validity while maintaining other metrics at the same level. Further controlling the model via WCEL was the key to this problem. Model performance was compared with a range of δ values in S.I. and choose δ=1 in WCEL. Next, different λ values were compared with δ fixed to 1. In FIG. 4, models are trained with the loss function [symbol] (λ, δ=1) throughout the training process for 200 epochs. FIGS. 4A, 4B, 4C and 4D show the performance comparison of the models trained with different λ values (and δ=1) using m-MMD loss: 906 L1D1; 907 L2D1; 908 L3D1; 909 L3.5D1; 910 L4D1. After each epoch, a sampling of 1k latent vectors was performed to evaluate NUV, and reconstruction rates were obtained from 1000 molecules from the validation set. It can be observed in FIG. 4A that higher λ values lead to better final validity. However, uniqueness in FIG. 4B breaks down for the case of when λ=4 while novelty and reconstruction rates converge to around 100% in (FIG. 4C and FIG. 4D). Therefore, the λ=3.5 model is trained for additional 200 epochs (total 400 epochs) to give final performance metrics in Table 3.

Generation with Beam Search

The model performance was further measured with beam search. A single output is selected from the B possible candidates with a beam size of B based on the criteria detailed in the decoding method.

Table 2 shows the model's generation performance with various beam sizes by sampling 10k latent vectors each time. One output is selected out of the interpretations given by all beam search results for each latent. Beam size of one is equivalent to not using beam search.

TABLE 2
Model performance with varying beam sizes

| Beam Size (B) | Novelty | Uniqueness | Validity | NUV |
|---|---|---|---|---|
| 1 | 0.996 | 0.974 | 0.998 | 0.968 |
| 2 | 1.000 | 0.996 | 1.000 | 0.996 |
| 3 | 1.000 | 0.998 | 1.000 | 0.998 |
| 4 | 1.000 | 1.000 | 1.000 | 1.000 |
| 5 | 1.000 | 1.000 | 1.000 | 1.000 |

Similar to [Jin et al., In International conference on machine learning, pages 2323-2332. PMLR, 2018; Richards et al., arXiv preprint arXiv:2205.01592, 2022] that applies checking methods to improve model performance, it was proposed to check the final generated results with a round of beam search. During molecule generation, one of the outputs was chosen from the B results returned in each beam search. For example, when using a beam size of two, for the same latent vector, two possible interpretations are produced. The B results were iterated through from the top one probable and if any SMILES is novel, unique, and valid, the check stops, and the SMILES is kept. Otherwise, keeping valid molecules is prioritized over unique and novel. Checking and selecting from all the beam-searched outputs increases the probability of finding SMILES' that are novel, unique, and valid. For each decoding, if all outputs are not valid, the top one scoring (summed log probabilities) result is returned.

In the case where the beam size is equal to one, the method is identical to greedy search which takes only the next-step candidate with maximum probability.

The results done at different beam sizes were compared. 10k vectors are sampled for each listed beam size. The result of the beam size of one is used as the control group.

Table 2 shows that with increasing beam size, the model's performance, measured in NUV scores, is improved. After using a beam size greater than three, the performance plateau at 1.0 which is the highest value possible for this metric.

Additionally, to demonstrate the beam search's capability, it was tested by sampling over a small distribution for a given latent vector. A molecule was chosen from the training set and it was encoded into its latent vector. This latent vector is added with 10 noise vectors that are individually sampled from a Gaussian with a tenth of the standard deviation (0.1-SD) relative to the one used in training. The noised latent vectors are decoded accordingly. The result from beam search shows diverse and similar candidates to the one being sampled around. 6 more candidates were found with the smallest scale beam search (beam size of two) compared to when beam search was not used (beam size of one).

### Generation Comparison

The generation performance is measured in terms of the novelty, uniqueness, and validity. The metric for generation alone is obtained by the product of the three (NUV). In addition, especially for VAE-like models, the ability to reconstruct is used to help finding similar candidates to the encoded ones. Therefore the reconstruction is taken into account when assessing model's overall performance using NUVR metric.

TABLE 3
The performance comparison between different molecular generation models trained with
ZINC250k dataset. The performance is measured in terms of both the models' abilities
to generate (NUV) and reconstruct (NUVR) without checking validity. Validity w/o
in the header means that the results have not been post-processed using chemical
knowledge to enforce corrections. Note that the disclosed NUV results are obtained
from averaging over 5 iterations of sampling 10k random latent vectors while the
reconstruction rate is calculated using all molecules from testing dataset.

| | | | Validity | | | | |
|---|---|---|---|---|---|---|---|
| Method | Novelty | Uniqueness | w/o | Validity | NUV | Reconstruction | NUVR |
| CVAE[6]^{a} | 0.980 | 0.021 | 0.007 | N/A | 0.0001 | 0.446 | 0.00006 |
| GVAE[7]^{a} | 1.000 | 1.000 | 0.072 | N/A | 0.072 | 0.537 | 0.039 |
| JTVAE[8]^{a} | 1.000 | 1.000 | 0.935 | 1.000 | 0.935 | 0.767 | 0.717 |
| MoFlow[12] | 1.000 | 0.999 | 0.818 | 1.000 | 0.817 | 1.000^{b} | 0.817 |
| Rebalanced[23] | 1.000 | 1.000 | 0.907 | 0.938 | 0.907 | 0.927 | 0.841 |
| GraphDF[13] | 1.000 | 0.992 | 0.890 | 1.000 | 0.883 | 1.000^{b} | 0.883 |
| ALL | 1.000 | 1.000 | N/A | 0.985 | N/A | 0.874 | N/A |
| SMILES[24]^{a} | | | | | | | |
| β-VAE[19] | 0.998 | 0.983 | 0.983 | 0.988 | 0.964 | N/A | N/A |
| KAE (This work) | 0.996 | 0.973 | 0.997 | 1.000 | 0.966 | 0.997 | 0.963 |

^{a}Results obtained from sampling 1,000 latent vectors.
^{b}Reconstruction rates calculated from the training dataset.

[6] Gómez-Bombarelli et al., ACS central science, 2018; [7] Kusner et al., pages 1945-1954. PMLR, 2017; [8] Jin et al., In International conference on machine learning, pages 2323-2332. PMLR, 2018; [12] Zang et al., In Proceedings of the 26th ACM SIGKDD International Conference on Knowledge Discovery & Data Mining, pages 617-626, 2020; [13] Luo et al., In International Conference on Machine Learning, pages 7192-7203. PMLR, 2021; [19] Richards et al., Conditional β-vae for de novo molecular generation. arXiv preprint arXiv:2205.01592, 2022; [23] Yan et al., Journal of Computational Biology, 2022; [24] Alperstein et al., All smiles variational autoencoder. arXiv preprint arXiv:1905.13343, 2019.

Constraint Optimization with CKAE (Similarity Search)

Following the benchmark by Zhou et al [Zhou et al., Scientific reports, 9(1):1-10, 2019], 800 molecules with the lowest P log P values from the ZINC250k dataset are chosen to perform the constraint optimization task where the optimized molecules are within 0.4 Tonimoto similarities to the original starting ones. The goal of this task is to find the molecules that will yield the largest improvement in the P Log P values within the allowed range. An equation by Gómez-Bombarelli and Jin was used [Gómez-Bombarelli et al., ACS central science, 2018; Wengong Jin, Regina Barzilay, and Tommi Jaakkola. Junction tree variational autoencoder for molecular graph generation. In International conference on machine learning, pages 2323-2332. PMLR, 2018] to calculate P log P:

$$P log P ( m ) = Log P ( m ) - SA ( m ) - ring ( m ) \qquad Equation 13$$

where, for molecule m, Log P is the octanol-water partition coefficient calculated with atom contributions using Crippen's approach [Wildman et al., Journal of chemical information and computer sciences, 1999]. SA is the synthetic accessibility score [Ertl et al., Journal of cheminformatics, 2009] and ring is the number of rings in the molecule that has more than six members.

The CKAE model trained was used on the same specifications as disclosed with λ and δ both set to one. During the decoding phase, all SMILES are allowed up to 190 maximum character lengths. Longer sequences are truncated.

Instead of using optimizers or regressors approach like [Jin et al., In International conference on machine learning, pages 2323-2332. PMLR, 2018; et al., arXiv preprint arXiv:2205.01592, 2022; Ma et al., In Proceedings of the 30th ACM International Conference on Information & Knowledge Management, pages 1181-1190, 2021; Yan et al., Journal of Computational Biology, 2022], a search procedure was developed called the Similarity Exhaustion Search (SES). The name comes from the action to perform repeated samplings across a range of conditions to obtain chemically similar molecules that are close to the target molecule in the latent space. SES is empowered by beam search and it has three hyper-parameters, the beam size B, interval δₛ, maximum increase in condition Δ, and number of repetitions in Phase-two R. In the disclosed implementation, parameters B=15, δₛ=0.1, Δ=20, and R=4 are used.

Condition Search: The SES initializes with each to-be-optimized molecule mᵢ and their P log P values as condition cᵢ where i is the index for the i'th molecule. The corresponding latent vector zᵢ is located by the encoder.

For step sⱼ, where j starts from zero, a search was performed for the vector zᵢ with condition cᵢ+jδₛ. The concatenated vector of zᵢ and its new condition vector were fed to the decoder. With beam search, B results were produced at each step. The procedures were repeated until jδₛ is equal to the maximum increment Δ. A total of

$$B + \frac{B Δ}{{δ}_{s}}$$

candidates were generated for mᵢ from this procedure. All candidates were filtered such that only the ones that were within 0.4 Tanimoto similarities were kept. The P log P values were calculated and ranked. The optimization was considered successful if the highest P log P value of the candidate for the i'th molecule was higher than its original value; The P log P value and corresponding candidate SMILES were then recorded.

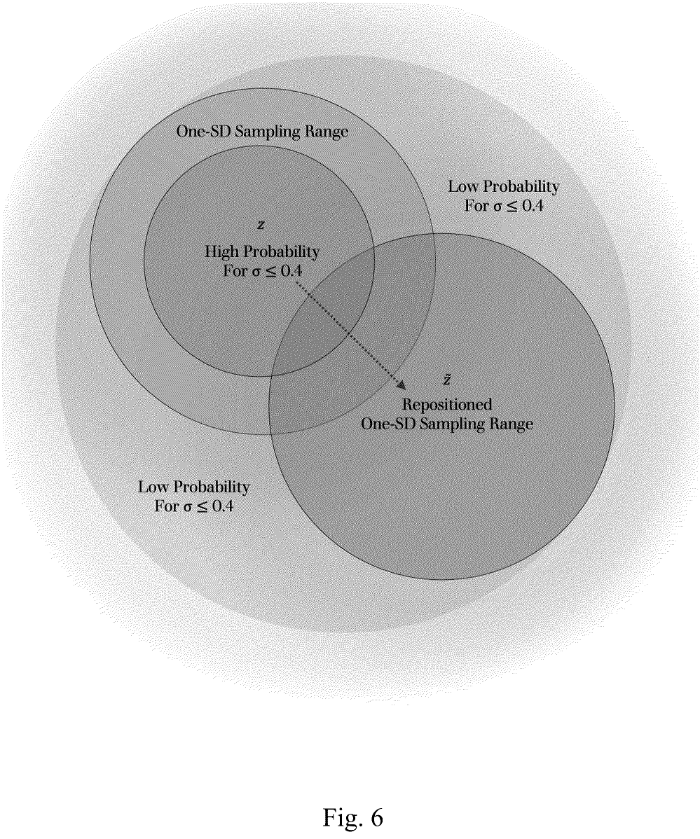

Repositioning: To encourage sampling further away from the encoded latent vectors sampling around zᵢ at cᵢ was performed after the condition search. The sampling was done by adding a noise e that belonged to the same Gaussian distribution used for training. If the sampled vector [symbol] produced a better result than the previous search, it was recorded. If there was [symbol] recorded, the next sampling would start from [symbol] . This step was to expand the exploration to molecules that were further away from zᵢ. It was especially useful for re-positioning vectors that had little or no improvements in the condition search. This repositioning procedure is repeated 100 times. A pictorial illustration of this procedure is shown in FIG. 6.

Phase Two: The condition search and repositioning resulted in two sets of latent vectors. One contained all originally encoded z; The other set included repositioned \tilde{z}. In phase two, the search was done in parallel with a combination of the condition search and repositioning. Each of the two sets was added noise the same way as in repositioning. However, each time this is done, every cᵢ is adjusted by cᵢ+jδₛ like in the condition search.

With the same filtering and selection criteria as in condition search, the new highest P log P-valued molecules were recorded for the two sets. Phase two was repeated for R times. After the R repetitions, for each molecule candidate, the better from the two sets was chosen and the final results were presented in Table 4.

In addition, to benchmark the model performance against the training data itself, a search within the training set ZINC250K was performed. For each of the 800 molecules, its similarity to all 250k entries was calculated in order to find the one with the maximum P log P value while staying within the 0.4 Tanimoto similarity constraint. This result is marked as ZINC250K in Table 4.

KAE has close to 100% reconstruction rate, and to demonstrate this advantage of being able to sample around accurately encoded vectors, a plain search with randomly sampled latent vectors at different conditions was performed. The condition P log P from −10 to 10 with step size of 0.1 was scanned across. At each step, 800 vectors are sampled from the latent space with each decoded using beam search with a beam size of 15. This procedure produced number of candidates equivalent to running phase two for 800 times for each molecule. The result of this search is marked as Random Search in Table 4.

All the disclosed errors denoted after the “±” in the Table 4 are standard deviations of the corresponding values. The improvements and similarities measure the mean difference in the P log P values and the mean of the Tanimoto similarity of the best candidate molecules and their starting molecules; The success rate measures the percentage of molecules that achieved modifications with higher P log P values within their similarity constraints. All constraint optimization results including the target molecules and the improved molecules' SMILES strings are provided in the SI.

### Docking Candidate Search

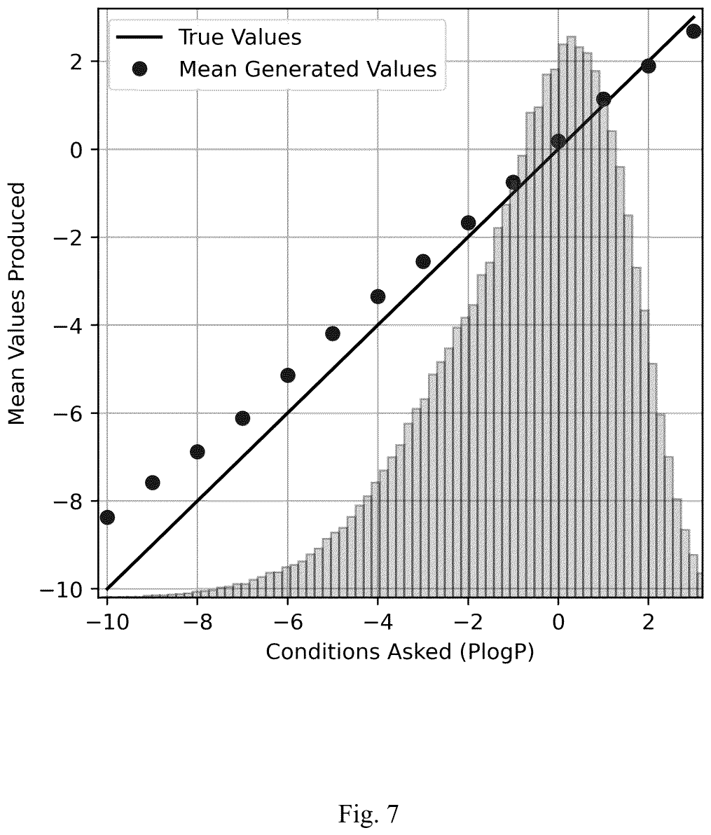

To examine the model for diverse applications, its ability to sample diverse molecules that can be useful for the task of docking was explored. Following approach by Bengio et al [Bengio et al., arXiv preprint arXiv:2111.09266, 2021], CKAE was trained on the selected dataset of 300k molecules where each of their binding energies are calculated from AutoDock [Trott et al., Journal of computational chemistry, 2010]. The energies are then converted by a custom scaling function, from the same source, to get to the metric called reward. Different from Gflownet, CKAE samples at all training conditions FIG. 7; By asking for larger conditions, unique, novel and better-binding molecules were sampled.

TABLE 4
This table compares different methods' results on constraint
optimization tasks. The performance is measured on the average
improvements for the set of 800 molecules. The success rate
is measured as the percentage of molecules that the algorithm is
able to improve. The ZINC250K is the result obtained by
searching for the highest PlogP improvement against the
ZINC250k dataset itself. Better results were demonstrated than
searching against the training data and the highest performance
was achieved using the disclosed model in combination with
the SES approach. (similarity constraint = 0.4)

| Method | Improvements | Similarities | Success Rate |
|---|---|---|---|
| JT-VAE[8] | 0.84 ± 1.45 | 0.51 ± 0.1 | 83.6% |
| MHG-VAE[29] | 1.00 ± 1.87 | 0.52 ± 0.11 | 43.5% |
| GCPN[30] | 2.49 ± 1.30 | 0.47 ± 0.08 | 100% |
| Mol-CycleGAN[1] | 2.89 ± 2.08 | 0.52 ± 0.10 | 58.75% |
| MolDQboot[25] | 3.37 ± 1.62 | N/A | 100% |
| ZINC250K | 4.64 ± 2.33 | 0.48 ± 0.16 | 97.88% |
| (This work) | | | |
| MoFlow [12] | 4.71 ± 4.55 | 0.61 ± 0.18 | 85.75% |
| Random Search | 4.78 ± 2.08 | 0.43 ± 0.03 | 81.75% |
| (This work) | | | |
| MNCE-RL[31] | 5.29 ± 1.58 | 0.45 ± 0.05 | 100% |
| β-VAE [19] | 5.67 ± 2.05 | 0.42 ± 0.05 | 98.25% |
| CKAE (This work) | 7.67 ± 1.61 | 0.42 ± 0.02 | 100% |

[1]Maziarka et al., Journal of Cheminformatics, 2020; Moret et al., Nature Machine Intelligence, 2020;
[8]Jin et al., In International conference on machine learning, pages 2323-2332. PMLR;
[12] Zang et al., In Proceedings of the 26th ACM SIGKDD International Conference on Knowledge Discovery & Data Mining, pages 617-626, 2020;
[19] Richards et al., Conditional β-vae for de novo molecular generation. arXiv preprint arXiv: 2205.01592, 2022;
[25]Zhou et al., Scientific reports, 9(1): 1-10, 2019;
[29]Hiroshi Kajino, In International Conference on Machine Learning, pages 3183-3191. PMLR, 2019;
[30]You et al., Advances in neural information processing systems, 31, 2018;
[31]Xu et al., Advances in Neural Information Processing Systems, 33: 8366-8377, 2020

To promote diversity in the candidates, beam search was not used for this task. 10⁶ NUV molecules were sampled and the rewards after Autodock Vina calculations were obtained. The average Tanimoto similarities are measured using a Morgan Fingerprint with a radius of two.

TABLE 5
This table compares docking candidates from GFlownet, training
sets and CKAE. The Top 10, 100, and 1000 rewards are the averages
of the rewards for molecules at the corresponding thresholds. The
Top-1000 similarity is the mean of all pair-wise similarities.
For diversity of the generated results, the lower similarity is
interpreted better, and for docking the higher rewards are better.

| | Top 10 | Top 100 | Top 1000 | Top-1000 |
|---|---|---|---|---|
| Method | reward | reward | reward | similarity |
| Gflownet | 8.36 | 8.21 | 7.98 | 0.44 |
| Training Data | 9.62 | 8.78 | 7.86 | 0.58 |
| CKAE | 11.15 | 10.46 | 9.63 | 0.63 |

The result of the mean reward for the top 10, 100, and 1000 best molecule candidates is listed in Table 5. CKAE sampled more similar molecules than reported by GFlownet. However, the rewards of the sampling candidate were considered better. In the training database, the maximum reward is 10.72 comparing to the maximum of 11.45 found from CKAE samples. This result shows the CKAE's extrapolation capability.

### Discussion for Constraint Optimization

CVAE interpolates the condition and produces molecules that have conditions correlating to the ones used as input. Each point in FIG. 7 is the average produced from 1,000 samples in the latent space. For every 1,000 samples, different conditions are attached. The corresponding P log P values are calculated from the SMILES output from the decoder. The correlation between the mean values produced from the model to the concatenated condition (condition asked) is 0.9997. This correlation to the asked conditions is affected by the underlying training data distribution. Better correlation is expected when training data is abundant and vice versa.

It is expected that with increasing P log P as a condition, corresponding molecules with properties close to the values that are asked can be produced.

The search using CKAE yields a better result than either the rudimentary ZINC250k search or the Random Search. Especially the Random Search samples approximately ten times more molecules per molecule target than in the CKAE Phase Two search. This is credited to the accurate reconstruction of the model. The encoder is able to determine the most accurate representation of the molecule in the latent space. This makes searching results around them much more efficient.

The purpose of condition search is to look for a set of candidates with similar encoder-estimated zᵢ but with higher P log P conditions. However, this procedure does not guarantee good samplings around some vectors as there were 8 molecules that were not exactly reconstructed. This means these molecules would have had starting points that make the decoded molecules dissimilar or even out of the similarity constraints from the encoded targets. Despite the correct reconstruction, because these molecules represent the tail of the distribution of the P log P conditions in the training data, they could have “rough” latent space around them. This can cause a similar problem to poor reconstruction where better candidates within the constraint cannot be found due to a decrease in either or a combination of validity, uniqueness, and novelty. Therefore, the resampling step is to ensure all molecules, especially for those zᵢ that cannot be reconstructed correctly, can explore possibly better-starting points in the later search (FIG. 6).

In phase two, the purpose of having two sets of latent vectors during the search is to ensure better and various starting points in the latent space.

It was found that the mean improvement from the z and 2 sets are 7.52 and 7.34 separately. However, by choosing the better of the two, the result is increased to 7.67.

### Discussion of Beam Search Results

With larger beam sizes, it is expected the output of the top one probable SMILES will have higher validity but lower uniqueness. This is likely because molecules with specific character combinations appear more often in the dataset. However, this is compensated with more possible candidates from the search results. The effect of beam search is better sampling efficiency which is not due to the sheer increase in the number of candidates. It was believed that the beam search can help differentiating two latent vectors that are similar by providing more interpretations per vector.

### Latent Space and Model Performance

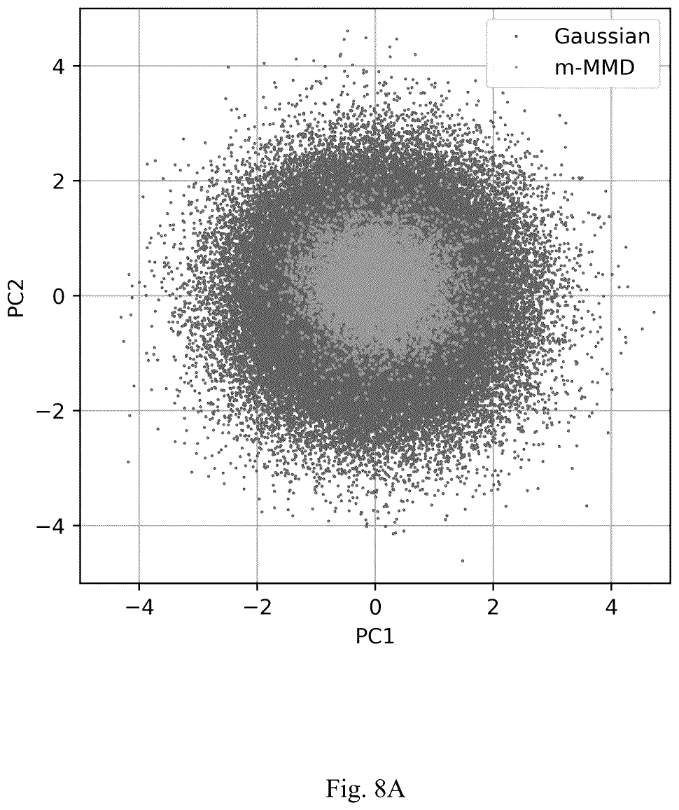

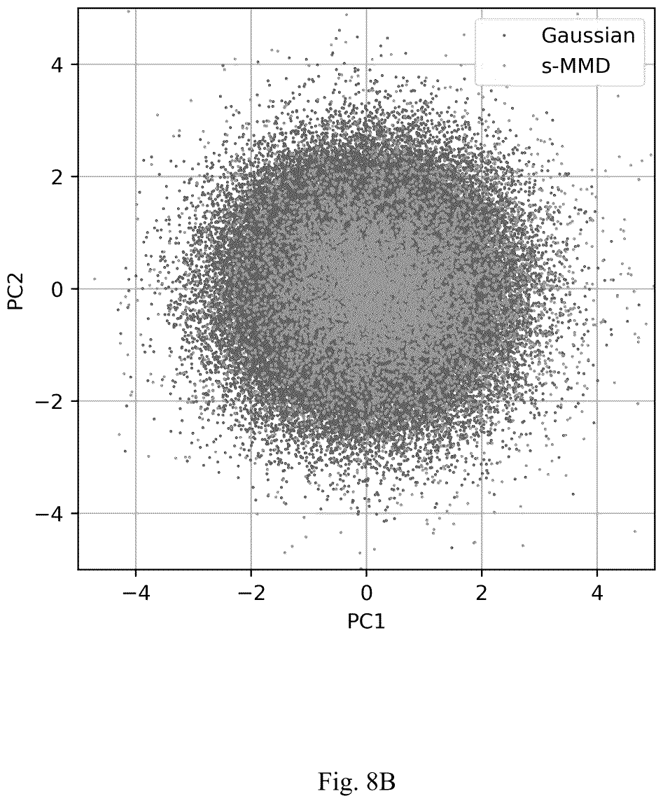

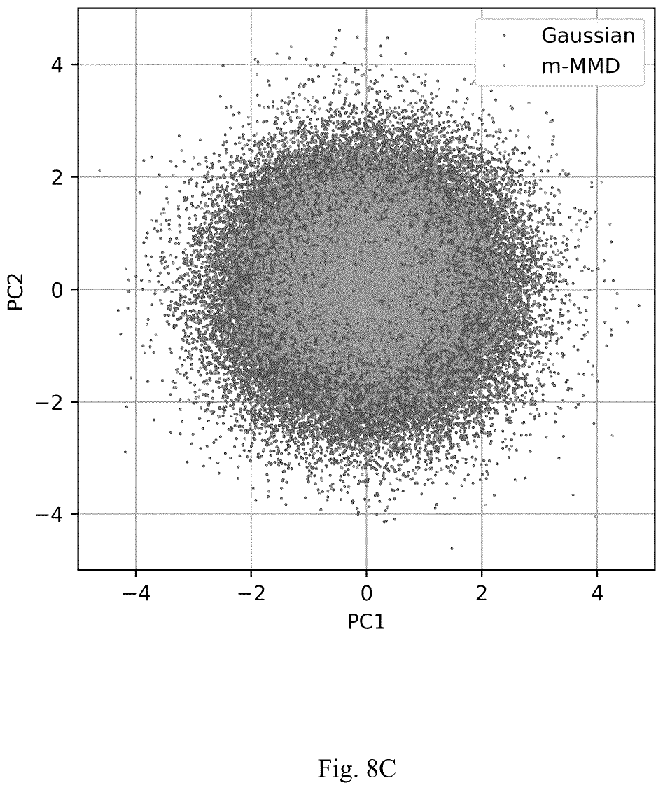

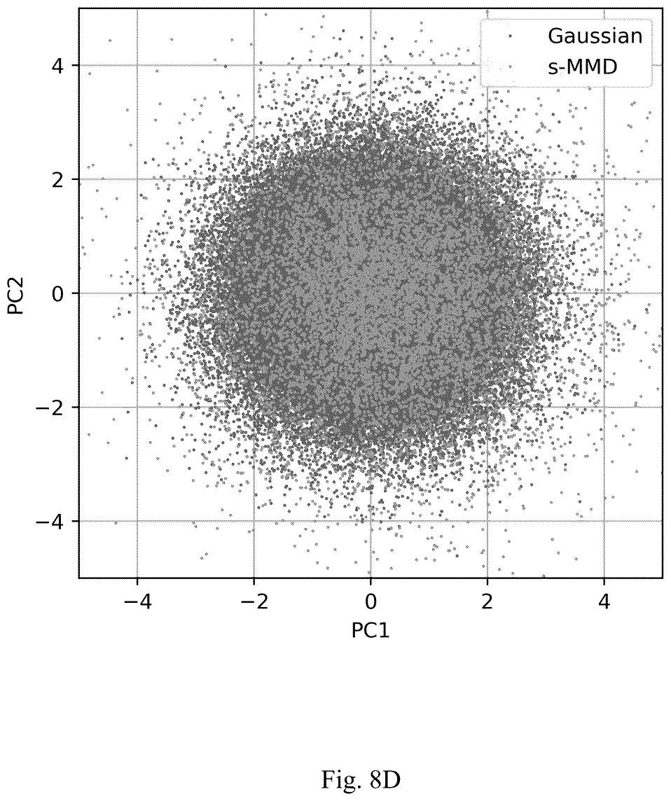

In m-MMD, with the RBF-kernel function, it was believed that removing the \vec{μₓ},^{T} \vec{μₓ} term is helpful since this allows the distributions of individual molecules to be closer together. This makes the sampling region have fewer places where the decoder cannot infer valid molecules. A demonstration and a comparison with the latent spaces of s-MMD and m-MMD are presented in FIG. 8.

The validity of the molecule is related to both syntactic and semantics. Syntactic correctness is to have the correct SMILES grammar; Semantic correctness means chemically meaningful. The probability P_{self} was denoted which depends on the decoder's ability to comprehend the SMILES and make the generated molecule both syntactically and semantically correct when given a latent vector outside of the training set.

The other relevant probability is referred to as Pₛₐₘₚ. Pₛₐₘₚ is the probability of sampling valid molecules in the latent space assuming the decoder is trained to recognize inputs from the target Gaussian distribution. All molecules can be interpreted as a “region” due to the addition of the Gaussian noise. When two or more regions overlap, a continuous interpolation between them can be generated. However, for example, when the sampled vectors land in the “holes” within the latent space or too far away from the target distribution, the model will be less likely to produce valid outputs. The sampling in latent space affects the output through the encoder-decoder attention.

To increase the probability of having valid outputs, the product of P_{self} and Pₛₐₘₚ should be considered together.

P_{self} term is learned by the decoder through the reconstruction process. The Pₛₐₘₚ, however, can be raised by scaling the λ parameter in the loss function in Equation 9 so that all latent vectors are more likely to be within the target distribution.

It can be seen from FIG. 3D that, with or without noise, the models trained with s-MMD have a faster-converging reconstruction rate than the models trained with m-MMD. This is because the extra [symbol] (\vec{x}, \vec{x}) term in s-MMD promotes the separation of the latent representations of the data points such that the decoder can easily differentiate the representations. However, since the latent vectors that represent valid molecules are far from each other, the validity is significantly lower for the models trained with s-MMD.

Increasing λ as an approach was considered to optimize the model performance in N, U, and V, reducing the regions with holes while still making individual molecules distinct from each other.

### Self-Optimizing Framework

As can be seen from both Vina and Glide results, CKAE produces better results than those in the dataset. It is therefore, possible to retain the higher-scoring data for new iterations of training. In this process, the model will be provided the information of better candidates and therefore more likely to produce even higher scoring candidates.

In conclusion, the disclosed architecture, Kernel-Elastic Autoencoder (KAE), represents a novel integration of the strengths of both Variational Autoencoder (VAE) and Autoencoder (AE) frameworks. By replacing the KL-loss with a Kernel-inspired loss in the KAE formulation, a flexible approach was offered that allows tuning of the model's characteristics using parameters λ and δ, incorporating varying degrees of VAE and AE features as needed. The disclosed method has wide-ranging applicability for problems that require both strong generation and reconstruction performances.

In the context of molecule generation, combined with its unique architecture and training procedure, KAE outperforms VAE approaches in terms of generation validity without the need for additional chemical knowledge-based checks, while achieving reconstruction performance akin to an AE, with close to 100% accuracy. The model's generative performance was further enhanced through the use of beam search, allowing for the identification of molecules that are not found otherwise.

Furthermore, the capabilities of conditioned KAE (CKAE) were demonstrated, trained on P log P values and docking scores, in finding superior candidates for constraint optimization and diverse search tasks. CKAE not only establishes a new state-of-the-art record but also outperforms searching from its training set by impressive margins of over 65% in constraint optimization and 6.8% in the docking candidate search. Importantly, the applicability of KAE and CKAE extends beyond the field of Chemistry, making them valuable tools for generation tasks and property-optimizing generation problems with pair-wise labeled training data in diverse domains.

In summary, the disclosed work presents a significant advancement in generative modeling, offering a powerful and flexible approach in the form of KAE and CKAE architectures. These findings highlight the potential of the disclosed models to bring new insights to molecular design and optimization and pave the way for future research in generative models with enhanced performance capabilities.

### Data Preparation

The ZINC-250K dataset was used consistent with [Rafael Gómez-Bombarelli, Jennifer N Wei, David Duvenaud, José Miguel Hernández-Lobato, Benjamín Sánchez-Lengeling, Dennis Sheberla, Jorge Aguilera-Iparraguirre, Timothy D Hirzel, Ryan P Adams, and Alán Aspuru-Guzik. Automatic chemical design using a data-driven continuous representation of molecules. ACS central science, 4(2):268-276, 2018]. During dataset preparation, all SMILES strings were added to the start of sequence tokens “<SOS>” and the end of sequence tokens “<EOS>”. The two tokens are used in the testing phase of the model to determine if the translation is completed. There were 41 unique characters from the database. They were extracted and put into a character-to-token dictionary that allows conversions from characters to tokens. The padding was added at the end as the 42nd token, making the dictionary size T. A token-to-character dictionary was created at the same time for the interpretation of the model output in tokens. With the character-to-token dictionary, all SMILES representations were converted to the corresponding tokens. Since the Transformer architecture was used, model inputs were made into the same shape for batch training by adding paddings to all sequences. After padding, all sequences have the same length. The numerical values of the penalized octanol-water partition coefficient (P Log P) were concatenated to the end of the corresponding tokenized molecules. This adds one extra dimension in the sequence length. The maximum sequence length for each molecule in the dataset is denoted as M. The tokenized dataset is then partitioned into 256-size batches.

### σ Comparison

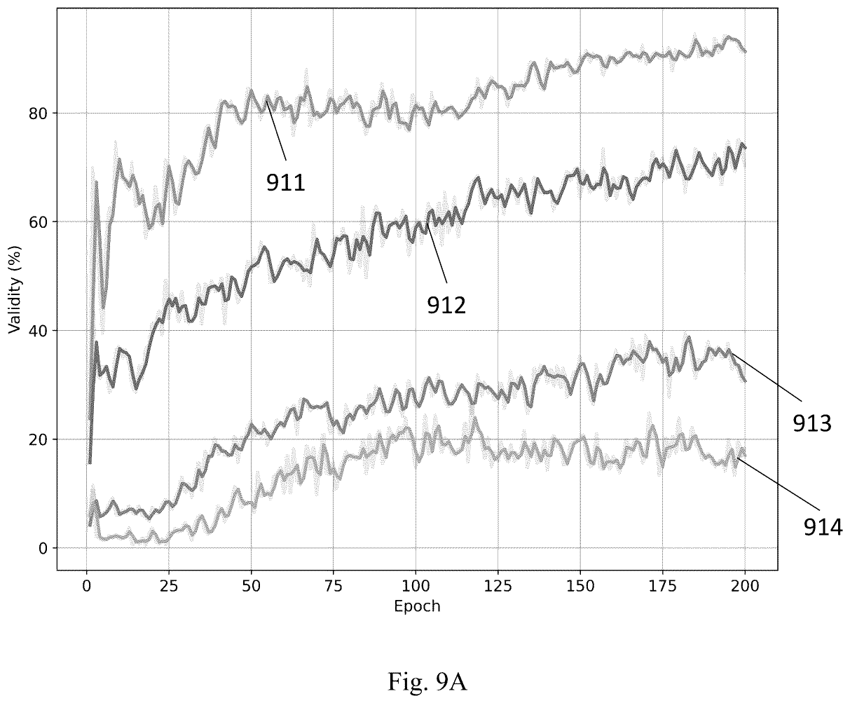

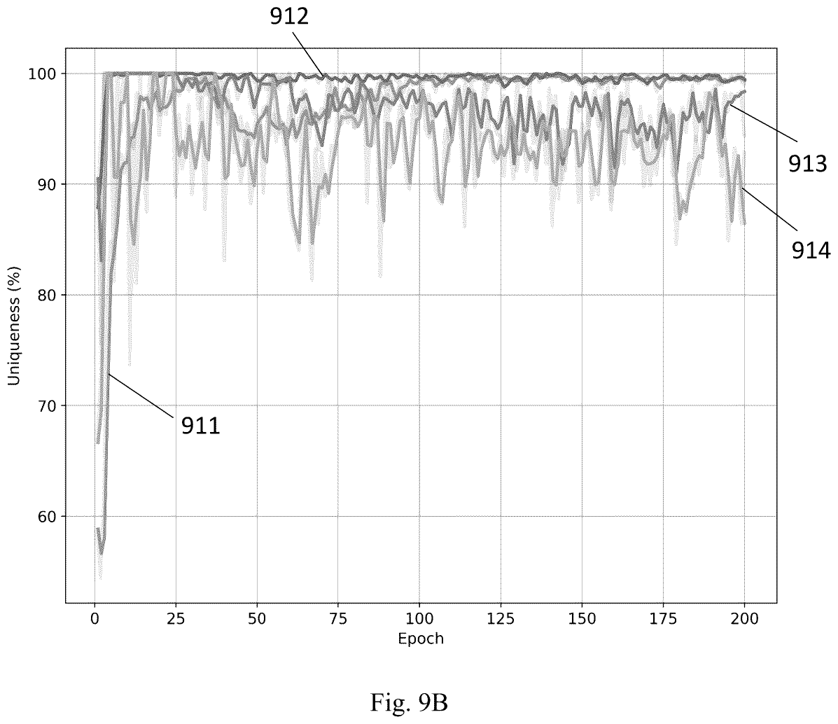

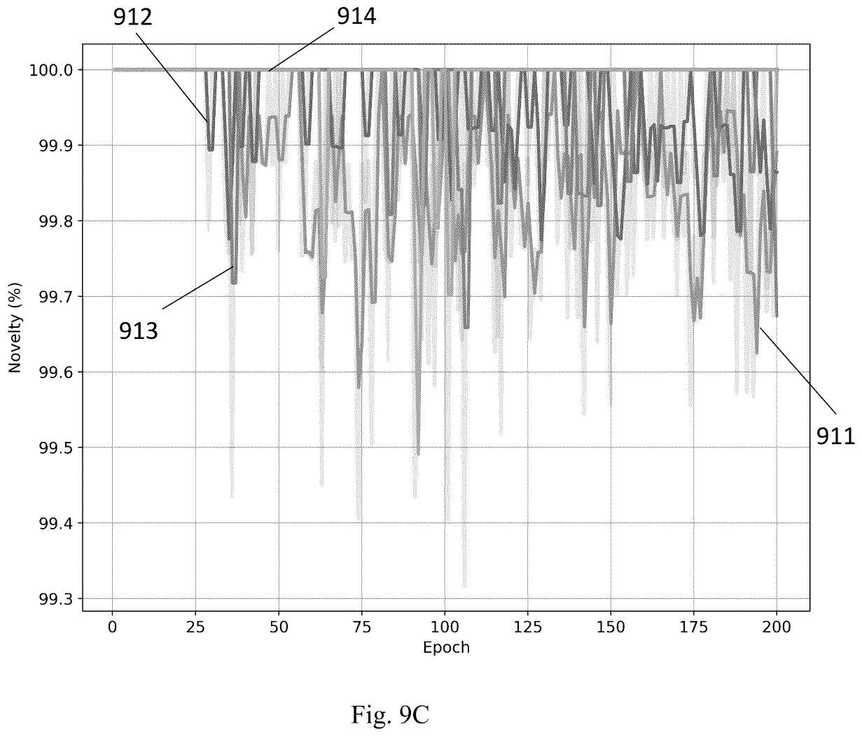

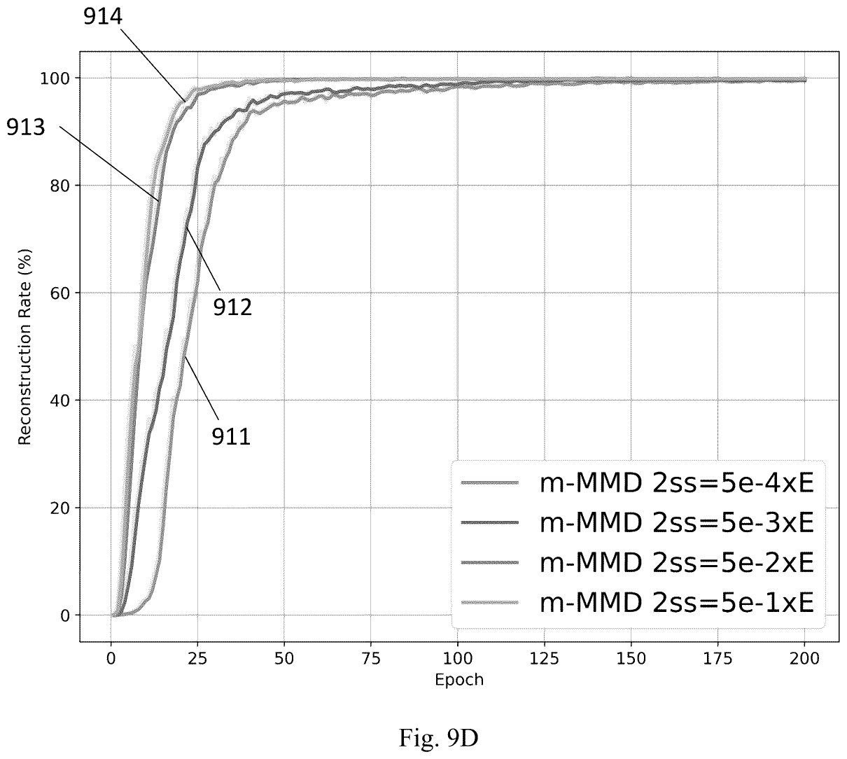

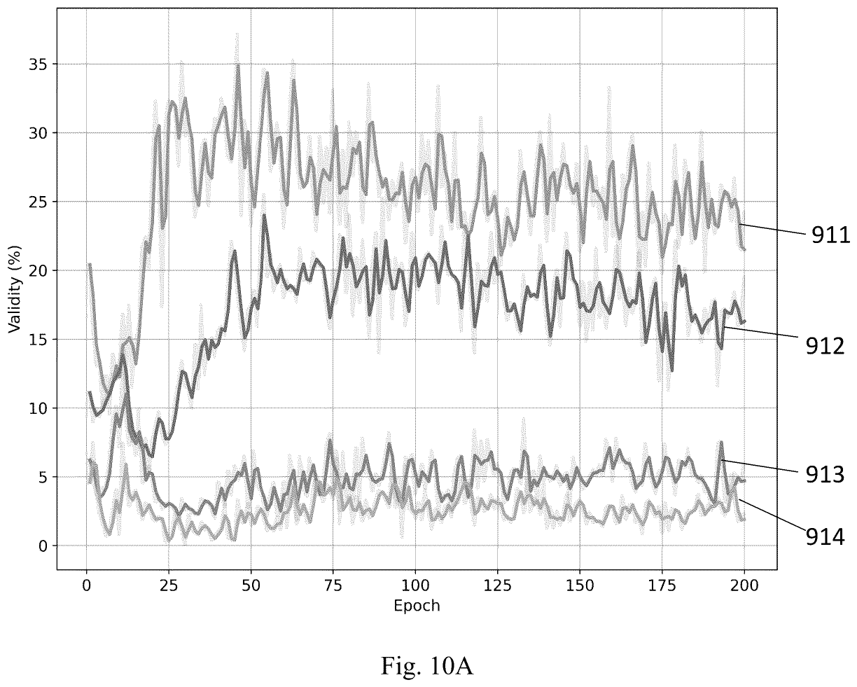

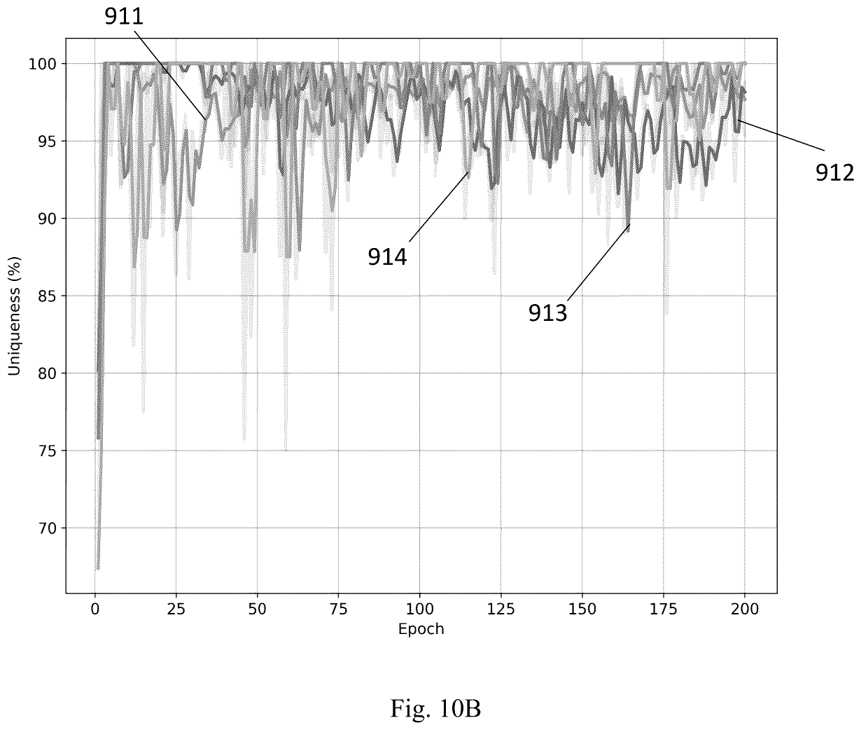

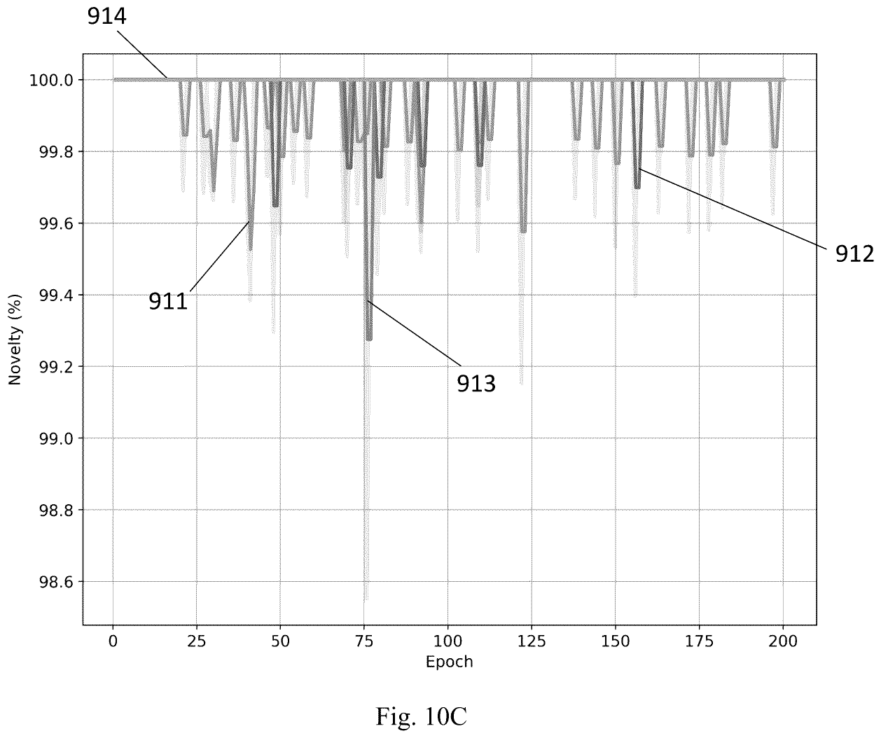

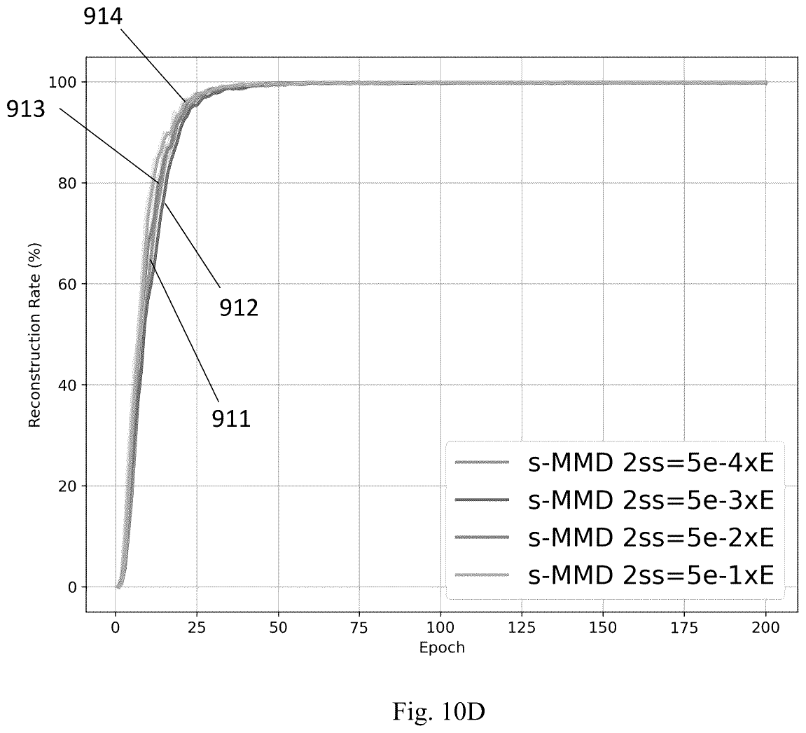

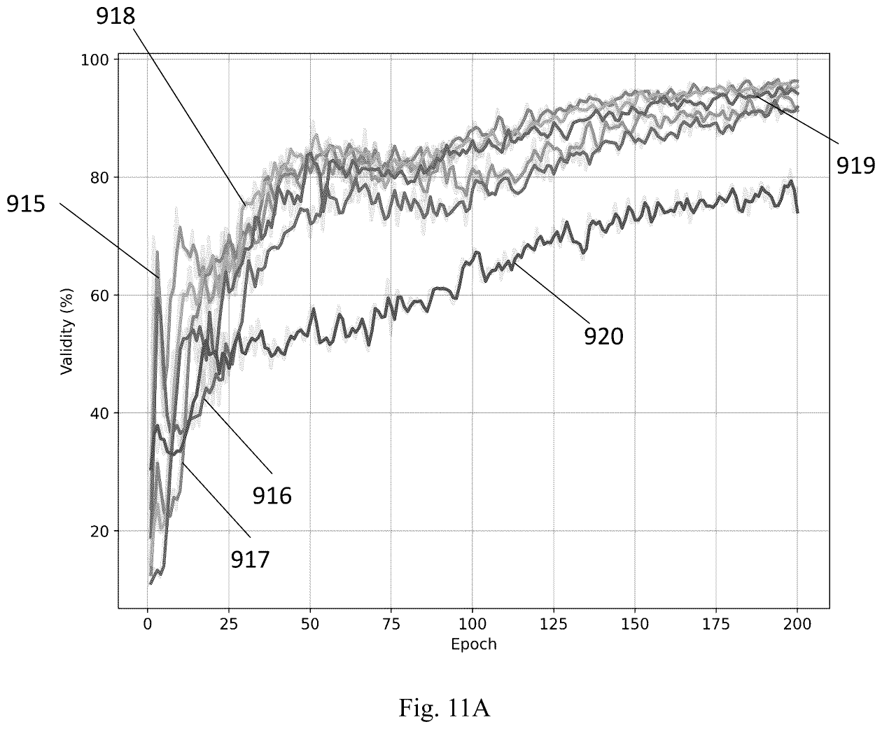

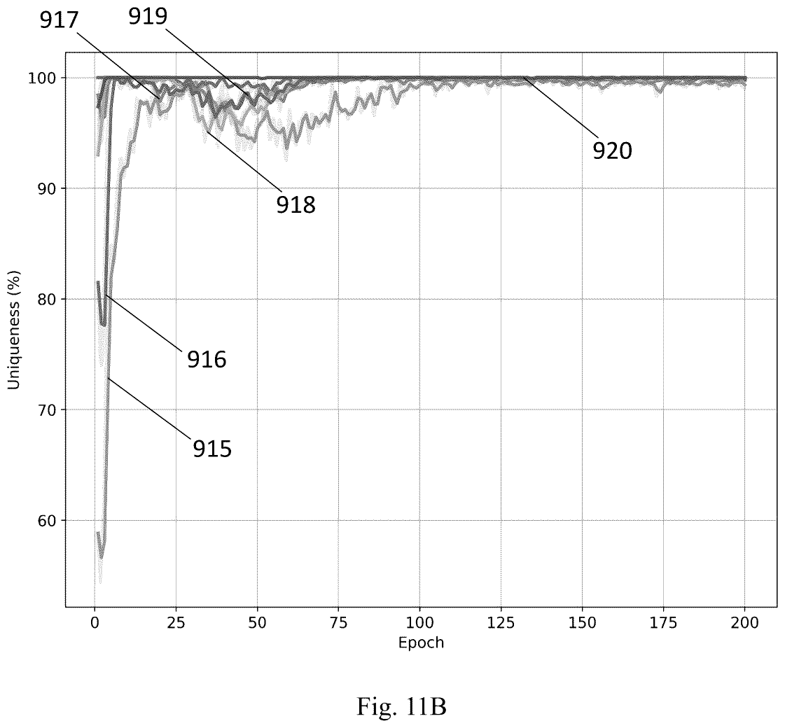

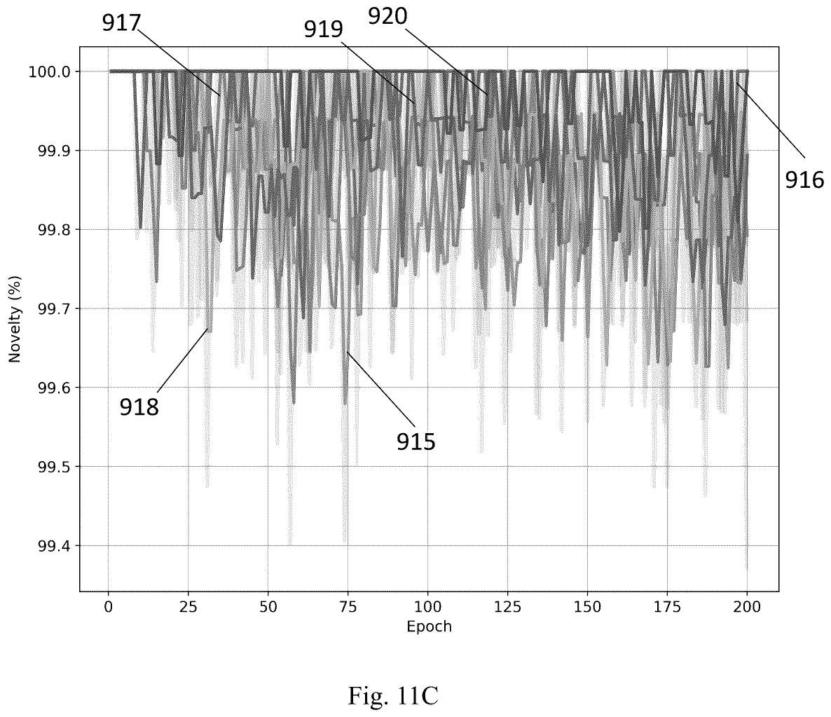

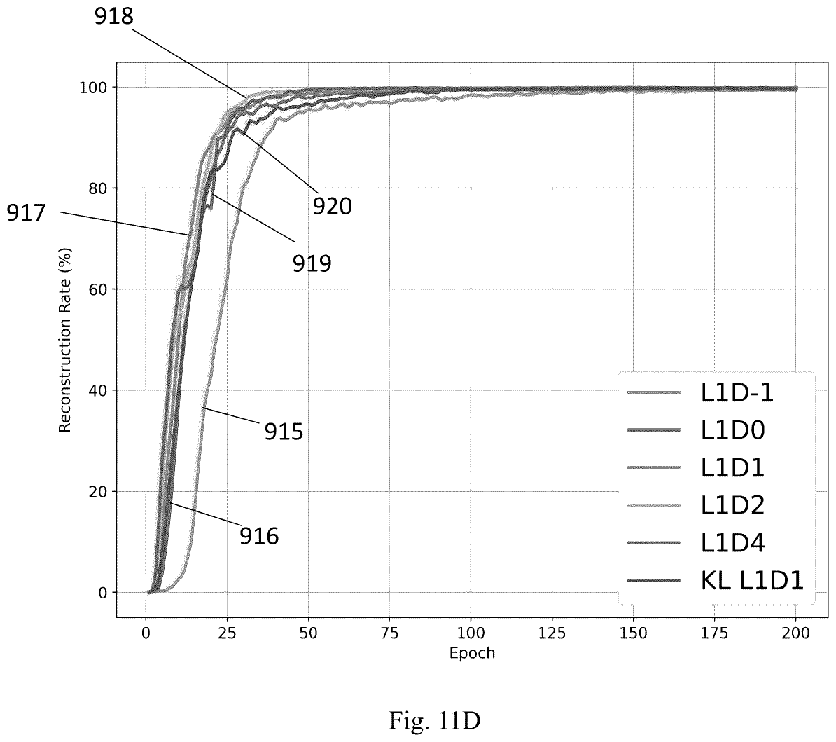

In FIGS. 9A through 11D, the model performance of different sigma values of the kernel was compared (Equation 3). FIGS. 9A, 9B, 9C, and 9D show the results for the performance comparison of the models trained with different sigma values using modified MMD loss: 911 m-MMD 2ss=5e-4×E; 912 m-MMD 2ss=5e-3×E; 913 m-MMD 2ss=5e-2×E; 914 m-MMD 2ss=5e-1×E. FIGS. 10A, 10B, 10C and 10D show the performance comparison of the models trained with different sigma values using standard MMD loss: 911 m-MMD 2ss=5e-4×E; 912 m-MMD 2ss=5e-3×E; 913 m-MMD 2ss=5e-2×E; 914 m-MMD 2ss=5e-1×E. FIGS. 11A, 11B, 11C, and 11D show the performance comparison of the models trained with different δ values (and λ=1) using modified MMD loss and KL loss: 915 L1D-1; 916 LIDO; 917 L1D1; 918 L1D2; 919 L1D4; 920 KL L1D1. It can be observed that the final uniqueness, novelty, and reconstruction rate are similar, while there are clear differences in validity performance. Therefore, the sigma value that gives the highest final validity rate is considered optimal. It can be observed that lower 2σ² values give higher validity rates and 2σ²=0.0005×E is the optimal value for both m-MM and s-MMD models. At the optimal sigma value, the m-MMD model has higher validity rate than the s-MMD model. Besides, if models are trained with even lower sigma values (2σ²=0.00005×E for example), the models would break down because they cannot get gradient information from the MMD loss term (results not shown).

### δ Comparison

The δ was designed such that when λ is 1, and δ is greater than −1, the AE-like term is contributing to how the model reconstructs the inputs. When δ is large, the model ignores the regions with added noise and thus is turned into a pure auto-encoder. When δ is equal to −1, the model is VAE-like where each latent vector is treated like a region. When δ is in between these two extrema, the model is able to achieve the AE-like reconstruction rate while obtaining better generative performance in NUV metrics. Finally, since the addition of δ is the key to the WCEL, the KL loss combined with WCEL was again compared with δ of 1, as opposed to the original CEL in VAE. It was shown that the disclosed formalism of the WCEL is capable of bringing significant improvements to KL-based models as well in FIG. 1A.

### Example 2: Retrosynthetic Prediction and Reaction Invention Using Conditional Kernel-Elastic Autoencoder

This paper presents a generative transformer model for retrosynthetic predictions, which comprises of a conditional kernel-elastic autoencoder (CKAE). The disclosed model uses a loss function and beam search procedure to ensure accurate reconstruction and diverse generation of templates for chemical reaction pathways corresponding to a target molecular product. Furthermore, the reactants corresponding to the generated templates are iteratively processed to generate complete, multi-step retrosynthetic pathways. To train the model, reaction templates are used to capture the reacting substructures in reactants and products. The results demonstrate the effectiveness of the proposed model in accurately and efficiently predicting retrosynthetic pathways.

Due to the vastness of chemical space and growing number of organic synthesis methods, manually designing molecule synthetic routes is becoming more and more challenging for experts because they might not be familiar with the compounds and available reactions. To address this issue, computer-aided technology has been developed to help this decision-making process since 1970s. However, these tools usually rely on hard-coded reaction rules so they often lack real-world applications. Recently, researchers have incorporated artificial intelligence (AI) or data-driven machine learning (ML) models into retrosynthesis tasks. These methods can be categorized as selection-based or generation-based. For the selection-based methods, researchers proposed reactant selection methods and template selection methods. Within generation-based methods, there are semi-template methods and template-free methods. The input and output of these methods can be molecular graphs, fingerprints, atom features, bond features, or strings such as SMILES or SMARTS.

Reactant selection methods [Guo et al., J. Chem. Inf. Model., October 2020; Lee et al., June 2021. arXiv:2105.00795] select molecules from a set of candidates given products as the input. The advantage is that the molecule candidates are always valid and can be chosen to be commercially available compounds. However, unless the molecule candidates include reactants in test set, the model would not be able to find the correct reactants. On the other hand, template selection methods [Segler et al., Chem. Eur. J., May 2017; Coley et al., ACS Cent. Sci., 3(12):1237-1245, December 2017; Ishida et al., J. Chem. Inf. Model., 59(12):5026-5033, December 2019; Fortunato et al., J. Chem. Inf. Model., 60(7):3398-3407, July 2020; Dai et al., January 2020. arXiv:2001.01408; Chen et al., JACS Au, 1(10):1612-1620, October 2021; Seidl et al., J. Chem. Inf. Model., 62(9):2111-2120, May 2022] provide the preference of reaction rules for given products based on a set of reaction templates. Reaction templates are subgraph patterns that capture the change in atoms and bonds for the reaction (find the reaction centers), and reaction templates can be extracted in terms of SMART strings by RDChiral [Coley et al., J. Chem. Inf. Model., 59(6):2529-2537, June 2019]. The advantages of template selection methods over reactant selection methods are that only single template has to be selected instead of multiple reactants and the coverage of reaction templates/rules is higher than that of reactants.

Semi-template methods [Yan et al., November 2020. arXiv:2011.02893; Shi et al., August 2021. arXiv:2003.12725; Somnath et al., June 2021. arXiv:2006.07038; Wang et al., Chemical Engineering Journal, 420:129845, September 2021] are generative-based. These methods first identify the reaction centers or rules then generate the corresponding reactants based on the given rules. While template-free methods [Liu et al., ACS Cent. Sci., October 2017; Karpov et al., preprint, Chemistry, May 2019; Chen et al., October 2019. arXiv:1910.09688; Lee et al., Chem. Commun., 55(81):12152-12155, 2019; Lin et al., Chem. Sci., 11(12):3355-3364, 2020; Zheng et al., J. Chem. Inf. Model., 60(1):47-55, January 2020; Tetko et al., Nat Commun, 11(1):5575, November 2020; Seo et al., AAAI, 35(1):531-539, May 2021; Mao et al., Neurocomputing, 457:193-202, October 2021; Sacha et al., J. Chem. Inf. Model., 61(7):3273-3284, July 2021; Mann et al., Computers & Chemical Engineering, 155:107533, December 2021; Ucak et al., J Cheminform, 13(1):4, December 2021; Kim et al., J. Chem. Inf. Model., 61(1):123-133, January 2021; Irwin et al., Mach. Learn.: Sci. Technol., 3(1):015022, March 2022; Zhong et al., Chem. Sci., 13(31):9023-9034, 2022; Ucak et al., Nat Commun, 13(1):1186, March 2022] address retrosynthesis tasks as sequence-to-sequence (sequences here can be represented as graphs as well) generation problems. Given products as input, template-free models have to reconstruct the corresponding reactants. These generation based methods have the possibility of inventing novel reactions since these models do not need to select from a set of candidates.

Here, a template-generation method was proposed where the generative model is trained to generate reaction templates instead of reactants. Unlike template-free methods, products are used as conditions and templates are used as input and output. This template-generation method inherit the template selection methods that have larger coverage than template-free methods since they are using reactants as output, while it is also able to search in space that cannot be accessed by selection-based methods and even invent novel reaction rules. With the generated templates and the “RunReactants” function from RDkit, reactants can be found from the given products. This also guarantees the validity of reactants just like reactant selection methods.

Conditional Kernel-Elastic Autoencoder (CKAE) is used as the ML architecture for this work where products are conditions and templates are the input and output. State-of-the-art performance of CKAE was shown on molecular generation tasks and therefore expect this architecture to work well for reaction templates.

### Reaction Template

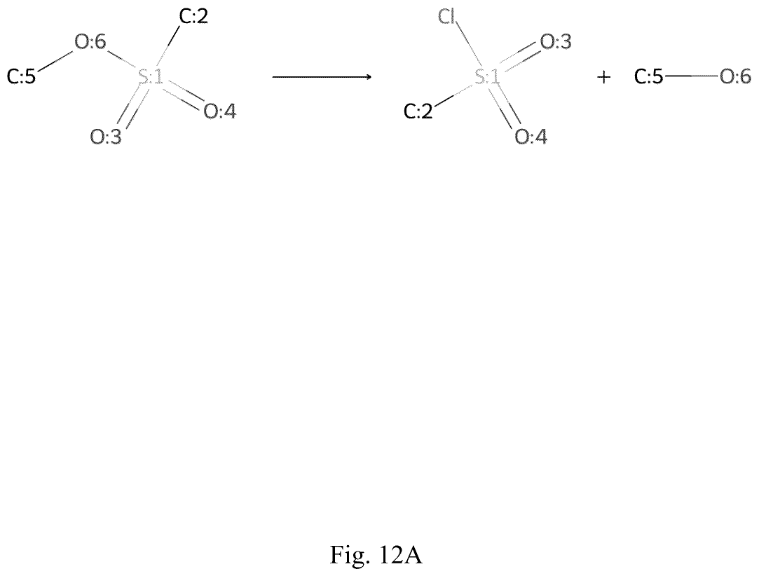

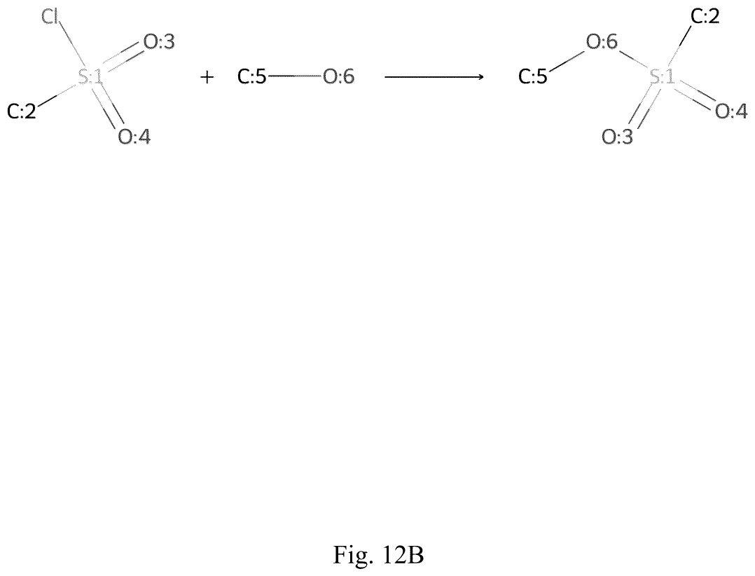

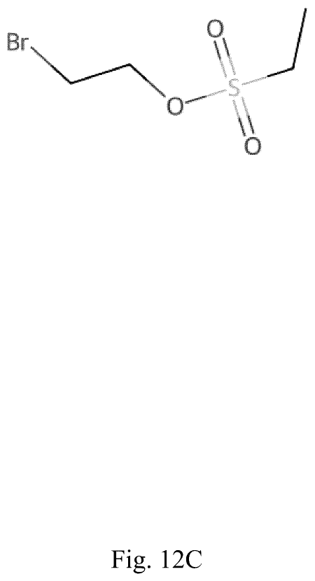

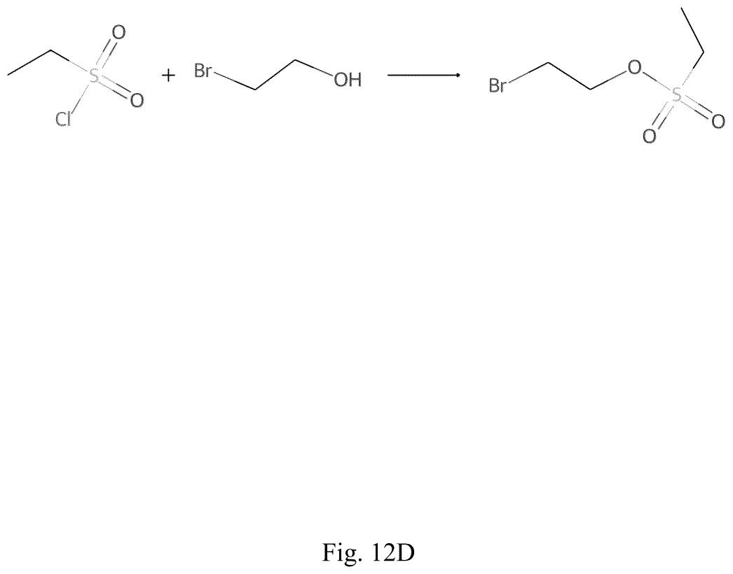

In this work, reaction template SMART strings are extracted from USPTO database in RDChiral. Example of a reaction template can be seen in FIG. 12. The original template in FIG. 12A gives the direction from product to reactants or the direction of retrosynthesis. If a molecule (product) with the substructure of the left hand side of the template is passed into the template, RDKit can output the molecules (reactants) on the right hand side. For example, the reaction in FIG. 12C can be obtained by passing the product in FIG. 12C to the template in FIG. 12A. Even though the original template strings are used for training, the format is not intuitive and convenient for readers. Therefore, the reversed format in FIG. 12B with the direction from reactants to product will be used to visualize templates and reactions for the rest of the work.

### Model Architecture for Template Generation

A conditional KAE was trained that is conditioned on products and the objective is to reconstruct the reaction template input for the given conditions. The trained model can thus be taking specific products as conditions and sample multiple novel single-steps from the latent space.

For the machine learning architecture, the same transformer encoder and decoder structures used in previous work was adopted. However, conditions are no longer values/properties for molecules, they are instead SMILES strings of products. Intuitively, the encoder structure was used as the disclosed conditioner to encode products as conditions that are concatenated with the latent space.

### Training Technique

In order to gain the best generation power while maintaining the reconstruction performance, λ=1 and δ=0 was chosen as the KAE training parameters. Similar to KAE for molecular generation, the generative power can be measured by novelty, uniqueness, and validity. Note that the validity definition for template SMARTS strings is slightly different from the validity for molecule SMILES strings. A valid template SMARTS string not only has to be a grammatically correct SMARTS string, but it also has to contain substructure of the given product condition. In other words, even though some generated templates can be visualized just like in FIG. 12B, the templates might not work for the specific products fed in the model conditioner. Given the success of masked-language modeling for BERT, masking was adopted to the disclosed encoder input (templates) while the decoder still has to reconstruct the unmasked strings. It was hypothesized that masking the whole product side (everything on the left side of >> in template SMARTS strings) would give us the highest generation performance because the latent space would contain almost no information of the product substructures, and the decoder would need the information from the conditioner to reconstruct the unmasked templates. This would give rise to better correlation between conditions and decoded templates and thus give better generation performance.

### Sampling for Single-Step Retrosynthesis

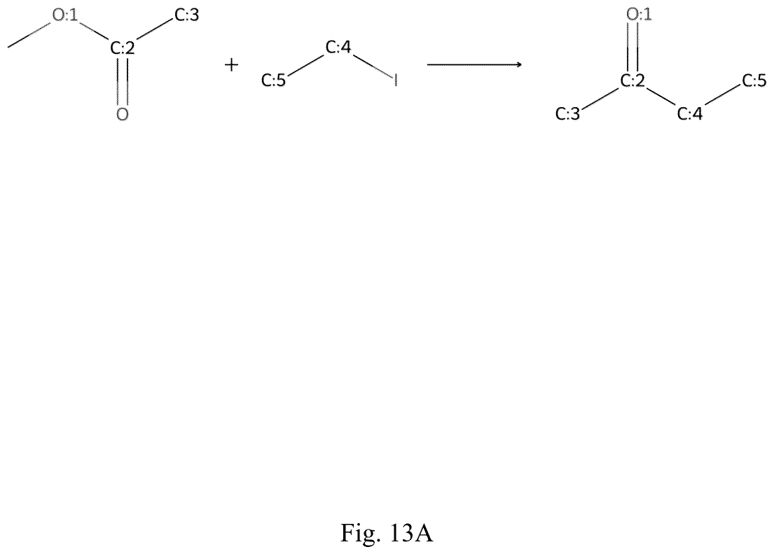

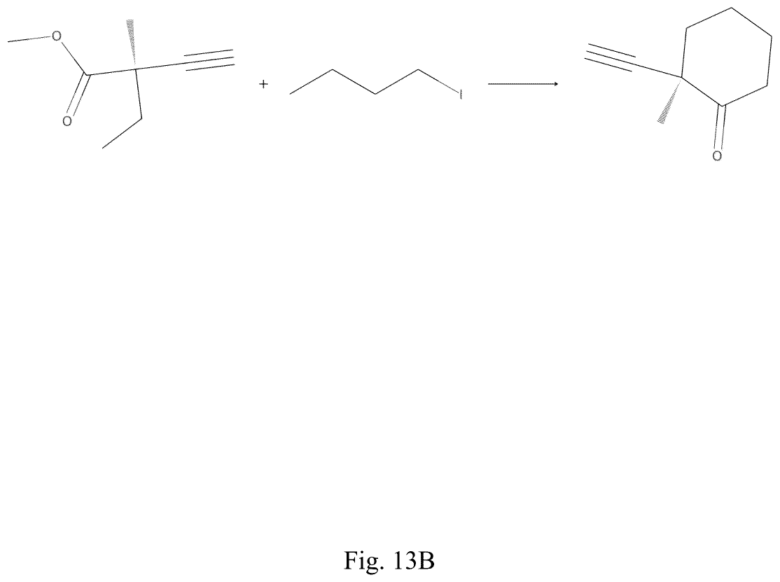

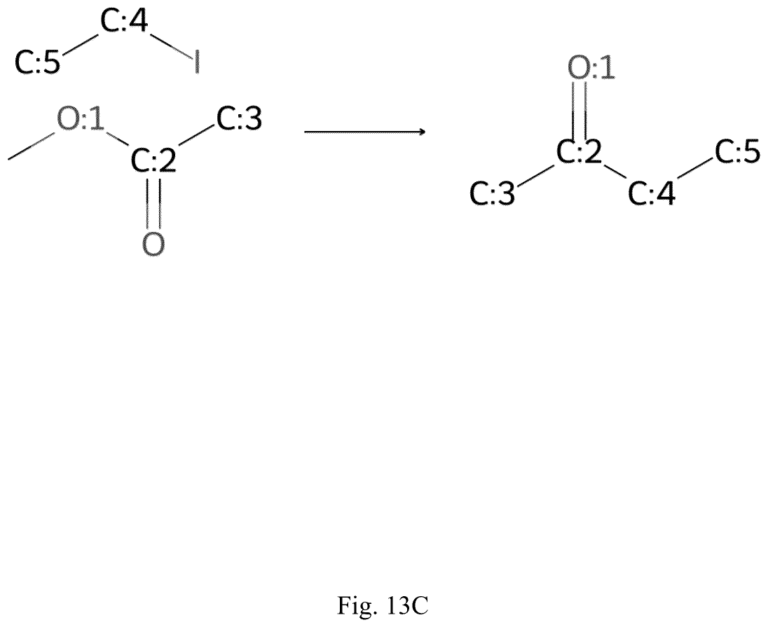

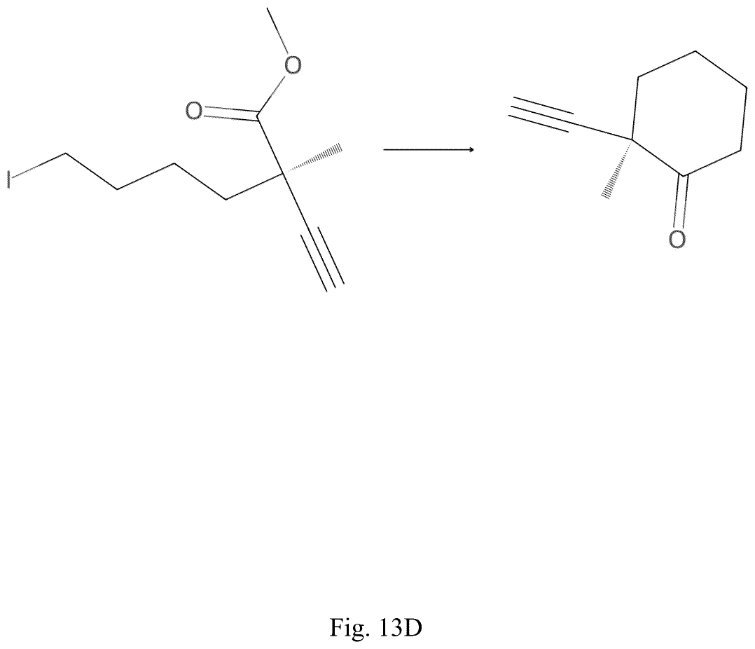

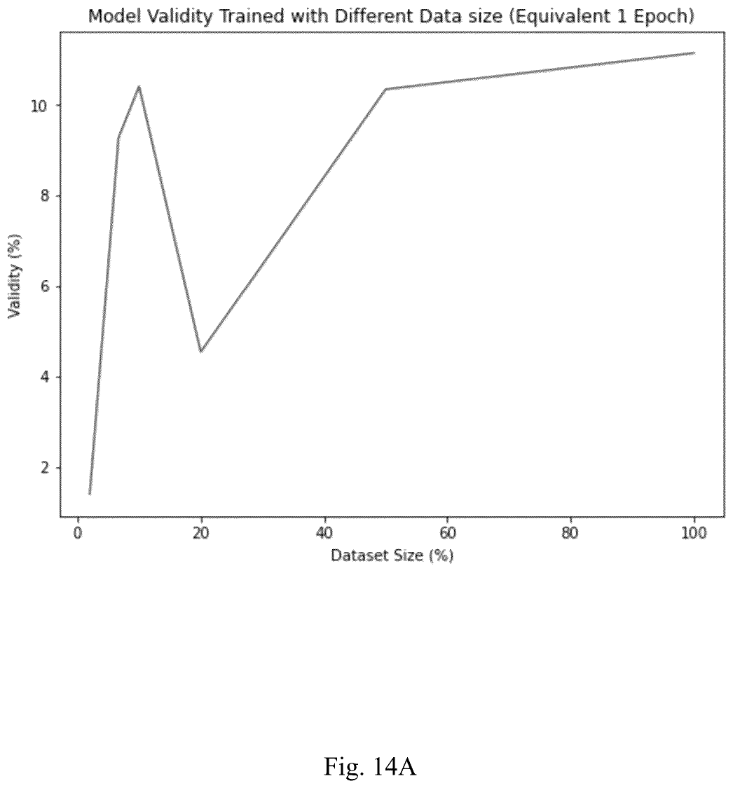

Since the decoder output is a probability distribution of the character tokens, adopt beam search was adopted to get more template strings from sampling. After the templates are sampled, as ring formation are of chemists' interest, whether there are ring number changes was monitored to determine if the reactions should actually be intramolecular. If there were ring number changes, parentheses were added on the reactant sides to define intramolecular reactions. An example can be seen in FIG. 13 where the sampled template in FIG. 13A can be converted to FIG. 13C by putting the reactant side of the template string (everything on the right side of >> in template SMARTS strings) between a pair of parentheses.

### Multi-Step Retrosynthesis

Most molecules cannot be synthesized in one step, so the disclosed single-step retrosynthetic prediction method was extended to an automated multi-step retrosynthesis application through beam search. First, a desired product is fed into the conditioner and several single-step templates can be sampled to obtain the reactants. A scoring function was then applied to find the best reactants. For these top reactants, they are fed into the conditioner again to get single-step templates for each of the reactants. This process just continues until a stopping condition like maximum number of retro-step, computation time, or commercially available precursors are found.

### Comparisons of Different Masking Strategies

TABLE 6
Performance comparison of different masking strategies. The
performance is measured by the number of valid and unique templates
generated by sampling 150 times using beam size of 10. Note that
Product Mask means that the encoder input templates are masked on
the product side, Reactant Mask means that the encoder input
templates are masked on the reactant side, and Random Mask means
that the characters of the whole strings are randomly masked.

| Method | Epoch 1 | Epoch 2 | Epoch 3 | Epoch 4 | Epoch 5 |
|---|---|---|---|---|---|
| No Mask | 67 | 180 | 165 | 210 | 218 |
| Random Mask (15%) | 64 | 155 | 181 | NA | NA |
| Random Mask (30%) | 72 | 115 | NA | NA | NA |
| Random Mask (50%) | 65 | 28 | NA | NA | NA |
| Reactant Mask | 145 | 167 | 134 | NA | NA |
| Product Mask | 167 | 241 | 266 | 275 | 302 |

The generative performance of different masking strategies for training was investigated. In Table 6, shown are the results of sampling 150 times with beam search of beam size 10 with different training strategies. It can be seen that masking the product side of the templates for encoder input has significantly higher rate of sampling unique and valid templates. Therefore, this training strategy was used for further testing.

### Novel Measures

New chemistry was defined as new types of bond connection/disconnection found on either side of the template. The design of a generative approach is opposite to having predictions only for the likely reactions but also to consider those that are less likely but novel. These reactions can be valuable, especially in the design of multi-step reactions which could have a significant reduction in the number of total steps by having one or few novel disconnections. It was first shown that the model learns about the task of retrosynthesis by benchmarking using the USPTO50k dataset. Then, the valid, novel, and unique connectivity obtained from the model was showcased. With the novel disconnections, the disclosed search space for possible next-step reactants is dramatically enlarged. To demonstrate this, the disclosed results were first benchmarked on the multi-step synthesis metrics, and the reduction in the number of steps was shown. Then experimental evidence was shown using the disclosed method which led to a reduction in the total number of steps required for synthesizing cyclohexanone.

### Why Generative Model

Generative model allows the mingling of different motifs which is not defined in the case of sampling by beam search. Generation from beam search does not lead to knowledge of the percent of possible outcomes explored until all possible outcomes are exhausted. A generative model with a well-defined latent space structure suffers less from such concern. Latent space has distance measures, which makes sampling around a target motif much easier than using beam search. Given a target template, beam search may only find variations of it without similarity constraints. Generative model can have similar types of outputs but different in terms of the sequence composition.

### Data Scalability

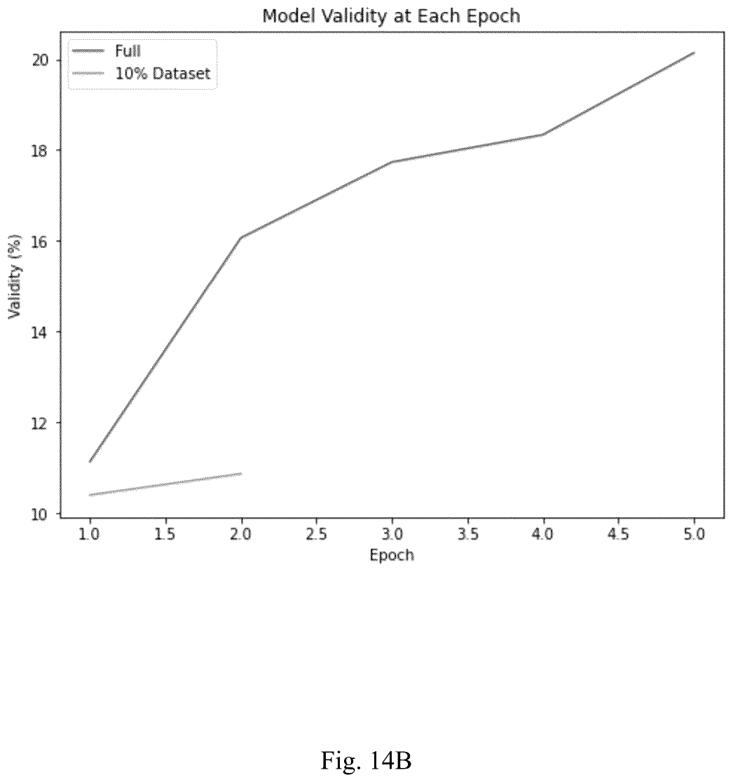

Given the success of product-masking models, the same model was trained with different training dataset size to research the data scalability. It can be seen in FIG. 14A that with 10% of the training dataset size, the model has reached the same unique and valid rate for sampling at 1 equivalent-epoch of training. However, with more epochs of training, the full dataset has better growth in performance. Therefore, it was predicted that the model can still be scaled significantly with larger dataset size. The current dataset size is around 1.5 million entries while Reaxys database has 34 million reactions.

Comparisons with Reaxys and SciFinder

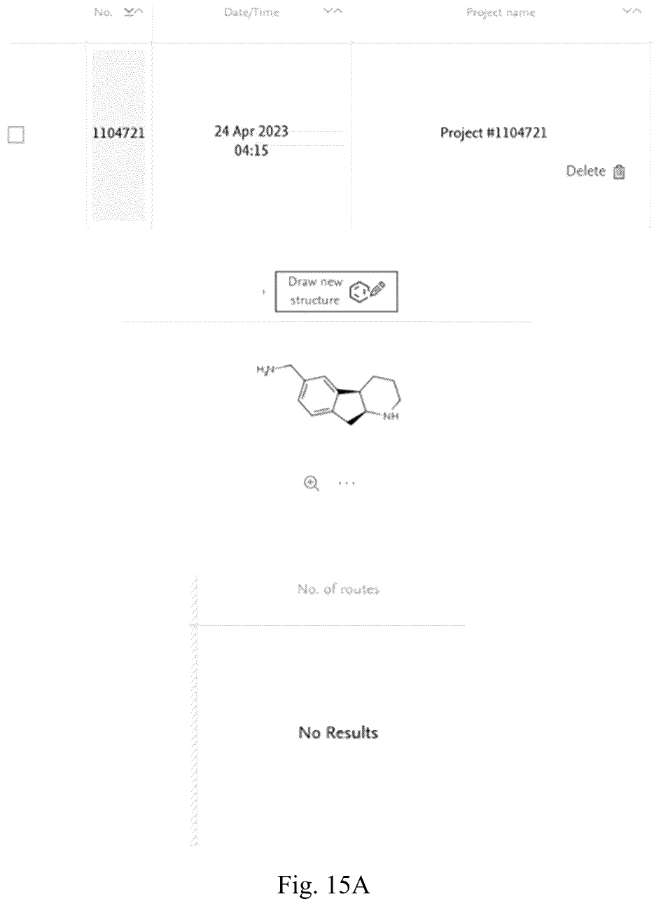

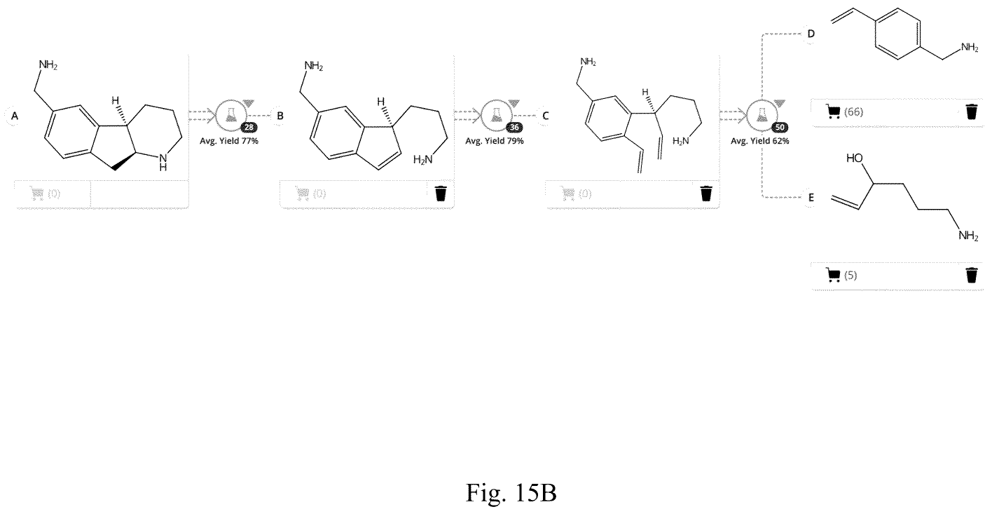

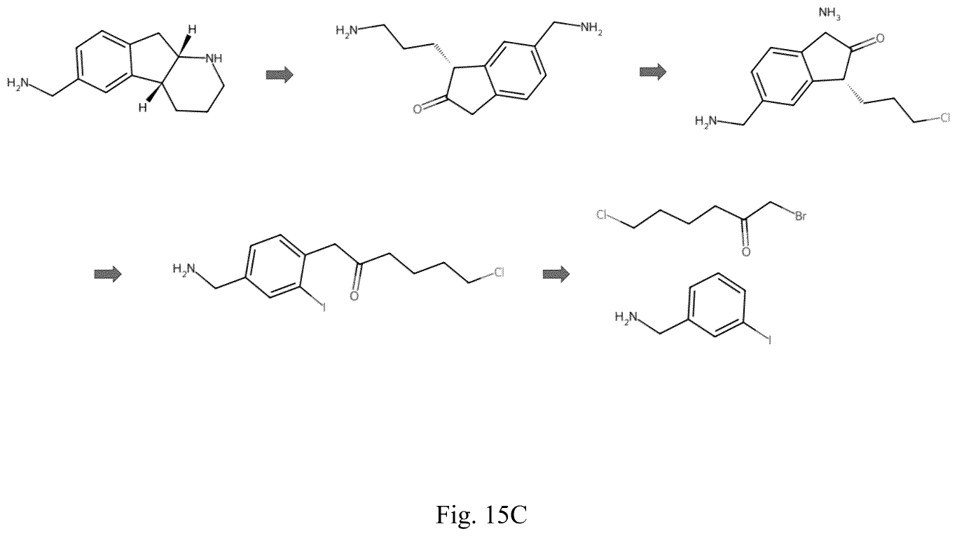

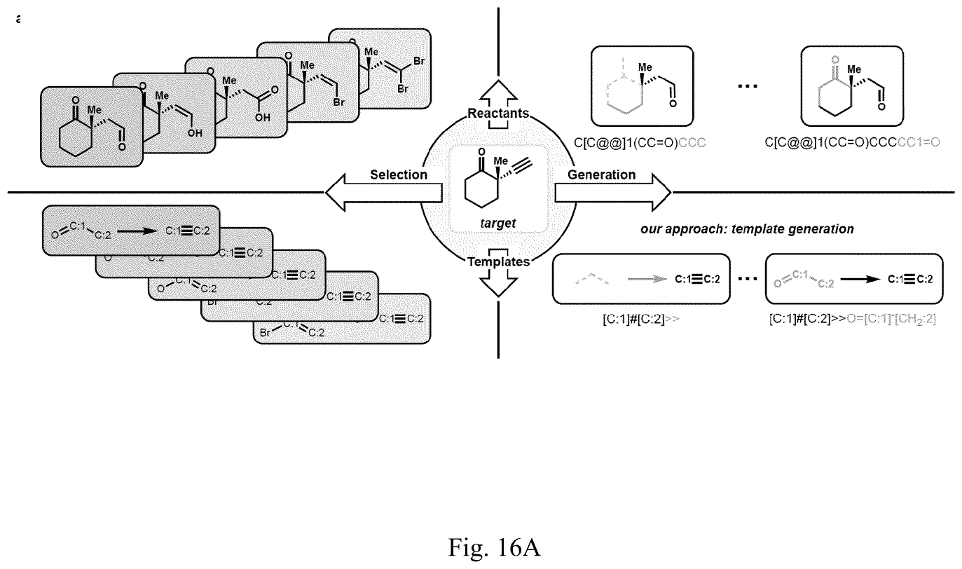

Here, the advantage of generative model over commercial retrosynthesis platforms, Reaxys and SciFinder was demonstrated in FIG. 15 for the same molecule (((4aR,9aS)-2,3,4,4a,9,9a-hexahydro-1H-indeno[2,1-b]pyridin-6-yl)methanamine, an intermediate of 11β-HSD inhibitor). It can be seen in FIG. 15A that Reaxys cannot come up with any routes for this molecule. SciFinder finds some routes after 40 minutes as shown in FIG. 15B. Interestingly, in just 5 minutes, the disclosed model has come up with more alternative reaction routes with one example shown in FIG. 15C. The results show the benefit of using this generative model comparing to methods using defined search space. This is because generative model can provide a more efficient search and explore in novel regions in the latent space.

A generative transformer model based on a loss function and a beam search procedure was designed that ensure accurate reconstruction as well as diverse generation of templates for chemical reaction pathways corresponding to a target molecular product. The reactants corresponding to the generated templates are iteratively processed to generate complete, multi-step retrosynthetic pathways.

### Example 3: Site-Specific Template Generative Models for Retrosynthetic Planning

Retrosynthesis, the process of designing synthesis routes for molecules by working backward from the target compound, has been a central focus of organic chemistry research. However, its effectiveness has been limited by the vastness of chemical space and the scarcity of training data. Disclosed herein is a new generative machine learning (ML) method for retrosynthesis planning: template generation. The disclosed approach includes three essential features. First, unlike previous generation methods that generate reactants or synthons, the disclosed models generate reaction templates. This approach enhances the level of abstraction in single-step retrosynthesis predictions. Second, a dedicated component in the disclosed methodology enables precise specification of reaction centers, granting control over molecular transformation sites. Third, generative ML models have intrinsic uncertainty in chemical feasibility for the generated reactions. Therefore, a separate model, powered by the conditional kernel-elastic autoencoder (CKAE) architecture, incorporates a latent space to provide a distance measure for reaction templates. This latent space distance measure allows for referencing generated reactions to reactions in the training dataset and provides valuable insights into chemical feasibility for the reactions. These features establish a coherent framework for retrosynthesis planning. In addition to building the ML models, a unique aspect of the disclosed work lies in the incorporation of experimental validation. Showcased herein is an ML-aided design of a complete retrosynthesis route that was subject to rigorous experimental testing. The successful reduction of synthesis steps in comparison to previous routes for the target compound from the experiments not only supports the model's robustness but also shows its potential for addressing a wide array of retrosynthesis problems.

Retrosynthesis is the design of breaking down complex molecules into simpler building blocks, a concept formalized by Corey and colleagues in the 1960s [E. J. Corey et al., Science, October 1969]. This laid the foundation for the development of Computer-Aided Synthesis Planning (CASP), a field that emerged to assist chemists in navigating various paths of synthesis. In the 1970s, tools like LHASA and SYNCHEM [W. Todd Wipke et al., AMERICAN CHEMICAL SOCIETY, June 1977; H. L. Gelernter et al., Science, September 1977] were introduced, relying on expert rules and heuristics to offer guidance. While these systems could not design entirely new reactions, they were invaluable in helping chemists overcome their inherent biases. In the 1980s, the IGOR software [Johannes Bauer et al., Tetrahedron Computer Methodology, 1988] utilized electronic redistribution patterns to discover novel reactions based on pattern matrices. By the 1990s, Hanessian and others [S. Hanessian et al., Pure and Applied Chemistry, January 1990] demonstrated the potential of collaboration between human expertise and machine guidance in proposing total synthesis routes, highlighting the inherent constraints of human relying on stored knowledge and precedents, and demonstrating how computers could complement these limitations.

Computational assistance in chemical synthesis is used to mitigate human bias and shortsightedness. However, early systems, rooted in expert rules, still carried traces of human subjectivity. Furthermore, as the field of organic chemistry flourished, the boundaries of known chemical space and synthetic methods are expanded. Expert rules struggled to keep pace with this ever-evolving chemistry. In more recent developments, CASP has transitioned from rule-based methods to precedent-based approaches [Orr Ravitz, Drug Discovery Today: Technologies, September 2013; Anders Bøgevig et al., Org. Process Res. Dev., February 2015]. This shift was facilitated by the large-scale extraction of reaction rules.

FIGS. 16A through 16C shows common machine learning methods for retrosynthesis and an exemplary approach. FIG. 16A shows reactants and templates can be selected and generated based on a target compound using different machine learning models. Template generation is used in the disclosed approach. FIG. 16B shows that latent space is incorporated in one of the models in the disclosed approach. Sampling in latent space can give different reaction templates. FIG. 16C shows a reduction of synthesis steps for a key intermediate for active pharmaceutical ingredients (API).

The process progressed from manual creation to automated extraction from extensive chemical datasets. Several extraordinary software had emerged due to this transition which empowered CASP tools to tap into vast repositories of historical reaction data [Haote Li et al., arXiv:2310.08685v1, October 2023]. Grzybowski and others [Chris M. Gothard et al., Angew Chem Int Ed, August 2012; Mikołaj Kowalik et al., Angew Chem Int Ed, August 2012; Tomasz Klucznik et al., Chem, March 2018] further introduced user-purpose-driven tools for route optimization, demonstrating remarkable success through experimental validations. Furthermore, the integration of machine learning (ML) methods has marked the latest chapter in the ongoing evolution of CASP. ML models offer promising alternatives and can be broadly categorized as selection-based, semi-template, or generation based methods [Zipeng Zhong et al., January 2023](see FIG. 16A).

Selection-based methods, such as reactant selection and template selection methods, aim to choose appropriate molecules or reaction rules from the given sets. Reactant selection methods [Zhongliang Guo et al., J. Chem. Inf. Model., October 2020; Hankook Lee et al., arXiv:2105.00795, June 2021] involve ranking molecules from a collection of candidates based on the target compounds. While reactant selection methods have the advantage of ensuring the chosen molecules are valid, their performance is impaired if reactants are not available in the candidate sets. Template selection methods [Marwin H. S. Segler, Chem. Eur. J., May 2017; Connor W. Coley et al., ACS Cent. Sci., December 2017; Shoichi Ishida et al., J. Chem. Inf. Model., December 2019; Michael E. Fortunato et al., J. Chem. Inf. Model., July 2020; Hanjun Dai et al., arXiv:2001.01408 [cs, stat], January 2020; Shuan Chen et al., JACS Au, October 2021; Philipp Seidl et al., J. Chem. Inf. Model., May 2022] rank the reaction templates in terms of their applicability to the target molecules. These templates capture subgraph patterns representing the change in atoms and bonds during a reaction. Notably, the RDChiral repository by Coley et al. [Coley et al., J. Chem. Inf. Model., 59(6):2529-2537, June 2019] offers template extraction methods and a collection of reaction templates in the form of SMART strings. Template selection methods offer distinct advantages over reactant selection methods; These methods simplify the reaction representation to a single template instead of multiple reactants. Additionally, the same template can be applied to different products/target compounds instead of having multiple sets of reactants for the target compounds, which provides a higher coverage of the reaction space. However, like reactant selection methods, template selection methods are also limited by the coverage and diversity of the available templates within the predefined reaction rules.

Semi-template methods [Chaochao Yan et al., arXiv:2011.02893 [cs, q-bio], November 2020; Chence Shi et al., arXiv:2003.12725 [cs, stat], August 2021; Vignesh Ram Somnath et al., arXiv:2006.07038 [cs, stat], June 2021; Xiaorui Wang et al., Chemical Engineering Journal, September 2021; Yu Wang et al., Nat Commun, October 2023]] involve the identification of reaction centers, synthons, or leaving groups, followed by the prediction of corresponding reactants based on these rules. Some semi-template methods [Vignesh Ram Somnath et al., arXiv:2006.07038 [cs, stat], June 2021; Yu Wang et al., Nat Commun, October 2023] are akin to selection-based methods, where reactants are obtained by predicting reaction centers and selecting from a collection of leaving groups. Other semi-template methods adopt generation components, in which reactants are generated from products and identified synthons or rules.

Generation-based methods is not bound by the sets of available reactants or templates. These include template-free methods [Bowen Liu et al., ACS Cent. Sci., October 2017; Pavel Karpov et al., preprint, Chemistry, May 2019; Benson Chen et al., arXiv:1910.09688 [cs, stat], October 2019; Alpha A. Lee et al., Chem. Commun., 2019; Kangjie Lin et al., Chem. Sci., 2020; Shuangjia Zheng et al., J. Chem. Inf. Model., January 2020; Igor V. Tetko et al., Nat Commun, November 2020; Seung-Woo Seo et al., AAAI, May 2021; Kelong Mao et al., Neurocomputing, October 2021; Mikołaj Sacha et al., J. Chem. Inf. Model., July 2021; Vipul Mann et al., Computers & Chemical Engineering, December 2021; Umit V. Ucak et al., J Cheminform, December 2021; Eunji Kim et al., J. Chem. Inf. Model., January 2021; Ross Irwin et al., Mach. Learn.: Sci. Technol., March 2022; Zipeng Zhong et al., Chem. Sci., 2022; Umit V. Ucak et al., Nat Commun, March 2022] that treat reactant generation as a translation task, aiming to predict the reactants directly from the given products without having in-dataset reaction rules. They therefore bear the potential to explore a wider range of possible reactions.

In the disclosed study, a new generation-based method to retrosynthesis planning that represents a distinct category is introduced: template generation. The conventional template-based methods have faced challenges. The process of constructing reaction templates often involves manual encoding or subgraph isomorphism which is computationally expensive [Connor W. Coley et al., Acc. Chem. Res., May 2018]. Template-based method's potential to explore reaction templates within the vast chemical space is often limited [Yu Wang et al., Nat Commun, October 2023]. To overcome these constraints, template generation models that employ Sequence-to-Sequence (S2S) architecture are trained to translate product information into reaction templates, as opposed to generating reactants. This method transcends the limitations of template selection-based approaches, enabling the discovery of novel reaction rules and expanding the scope of retrosynthesis planning. The combination generated reaction templates and the “RunReactants” function from RDKit, offer an efficient means to swiftly identify templates that yield grammatically coherent reactants from given products. This facilitates the exploration of previously uncharted chemical reactions and pathways.

One of the major benefits of using the reaction template is the ease of checking reaction validity. During the transformation of a reaction template, the product is guaranteed to be converted to the reactant with exact matching of atoms indices and relevant functional groups from the description of template. In comparison to reactant generative models, this benefit greatly reduces the uncertainty in the produced reactants which might not correspond to any known reactions or have key atom mismatches due to problems during decoding.

The second design is a sampling generative model (sampling model) for template generation that applies to a target product. S2S models, such as those employed in the template-free methods, predict retrosynthesis results deterministically and do not have a sampling process or definition of latent space. In contrast, the disclosed sampling model has a latent space, enabling the generation, interpolation, and distance measurement of various templates (FIG. 16B). Deterministic models that takes target compounds as input and generates templates are also developed in the disclosed work. Importantly, the encoder of the model can incorporate positional embedding for reaction centers, enabling users to specify specific reacting sites during prediction where the results are benchmarked on the USPTO-FULL dataset.

The disclosed sampling model based on the conditional kernel-elastic autoencoder (CKAE) [Haote Li et al., arXiv:2310.08685v1, October 2023] is the first of its kind in the field of retrosynthesis. This model conditions on corresponding products during training, allowing interpolating and extrapolating capabilities of reaction templates in the latent space to generate templates during the sampling process. The latent space also provides a measure of distances between reaction templates, allowing means to identify the closest reaction reference within the dataset or determine the similarity between two reactions.

The disclosed template generation method introduces a special design where the templates, which are referred to herein as site-specific templates (SST), exploit just the reaction centers. This results in a concise and informative set of templates different from the templates available in the RDChiral repository [Connor W. Coley et al., J. Chem. Inf. Model., June 2019]. Additionally, SSTs and target compounds with reaction centers labeled (center-labeled product, CLP) are simultaneously encoded/decoded, allowing the model's attention mechanism to incorporate reaction centers defined by atoms in the molecule context. Integrating these features into the template generation process ensures the relevance and practicality of the generated templates.

Through benchmarking with public dataset, the efficiency of using the template-generative model for robust retrosynthesis prediction with highly flexible reaction center controls is demonstrated herein. In addition, to resolve the common problem of having new unidentified reactions, CKAE's latent space is used to establish distance measurement which allows the referencing of reactions within the training set.

With SSTs and generation methods in place, the disclosed approach was validated through the practical application of synthesis. Compound lb-7 was reported by Boehringer Ingelheim [Jason ABBOTT et al., U.S. Patent 2023/0212164 A1, 2023] along with a library of analogs, as a potent Ba/F3 KRASG12C inhibitor, and potential anti-cancer agent. The synthetic route for lb-7 has two key intermediates (FIG. 16C), a thiophene derivative and its precursor compound 1. A cyclohexanone with quaternary chiral center in α-position containing alkyne moiety is considered a synthetic challenge. ML model coupled with human intuition is used to determine the most step-efficient way to synthesize compound 1, thereby reducing the number of steps from 5 to 3 compared to previous work [Jason ABBOTT et al., U.S. Patent 2023/0212164 A1, 2023]. The disclosed experimental validation provides insights into the practicality and reliability of retrosynthesis predictions, reinforcing the models' robustness and their underlying promise to address a wide spectrum of retrosynthesis problems.

### Results and Discussion—Site-Specific Templates and Center-Labeled Products

Disclosed herein, reaction templates and reaction centers are analyzed. In the disclosed work, templates are utilized as concise representations of chemical reactions, capturing substructure changes during reactions. The focus of this study is on templates that only apply to reaction centers within the target compounds, referred to as site-specific templates (SST). This distinguishes this work from RDChiral templates, which encompassed a broader structural context. This distinction is crucial, as SSTs do not take into consideration of neighboring atoms and special functional groups when matching substructures within the target compounds. The presence of center-labeled products (CLP) is a prerequisite for the effective use of SSTs. Since SSTs could potentially be applied to multiple sites within target compounds/products, SSTs may result in ambiguity without such labeling. Examples of SST and CLP are shown in FIG. 16D.

### Deterministic Model Performance

Deterministic generative models, such as those found in previous template-free methods, adopt a deterministic approach for generating templates without relying on a latent space for sampling. In contrast to generative models that employ latent sampling methods, deterministic models focus on proposing viable reactions based on a given product. FIG. 16D illustrates the workflow for the disclosed deterministic models: Model A and Model B. Model A takes target compounds as input and passes them through a encoder-decoder architecture, which translates targets into SSTs and CLPs. CLPs unambiguously specify the application of SSTs on target compounds. Model B, instead of outputting CLPs, takes in reaction centers of the target as positional embeddings.

A comparison of Top-K accuracy between the disclosed deterministic models and other methods are presented in FIG. 16E. The Top-K accuracy is the percentage of top K predictions that precisely match the correct reactants from the test set within K predictions. Both accuracy results for original and cleaned test set are presented. Cleaned test set is introduced as problems related to atom-mapping issues in the USPTO-Full dataset are encountered. The issues result in solvent and reagent atoms erroneously considered as part of the templates. To address this problem, reactions containing the 50 most frequently observed spectators in USPTO-FULL as participating reactants are removed from the test set. This removal process led to a decrease in the test set size to 90.7% of the original size (originally 95k reactions), which resulted in improved Top-K accuracy. See below for details of how beam search is used to obtain Top-K results.

Model A, which does not use reaction centers, performs comparably to other methods using the original test set. The removal of the 50 most common spectators for the cleaned set largely improves the accuracy, but it may inadvertently exclude some reactions where these common spectators actually participate as reactants. Unfortunately, due to the absence of a systematic approach for identifying and removing incorrectly labeled reactions, this pragmatic solution is used. In contrast, Model B leverages reaction center information. On the cleaned set, Model B reaches a significant performance milestone, achieving an accuracy rate of 80% for Top-10 predictions.

RetroExplainer [Yu Wang et al., Nat Commun, October 2023], with semi-template components, has remarkable prediction accuracy owing to its data modeling approach and the utilization of a set of leaving groups. Nonetheless, this approach may encounter variations in performance when dealing with uncommon scenarios or leaving groups that are not explicitly represented in the dataset. Its capacity to generalize to novel situations may be constrained. R-SMILES [Zipeng Zhong et al., Chem. Sci., 2022], a template-free generation-based method, introduced the root-aligned SMILES representation to ensure minimal edit distances between product and reactant SMILES. With this custom string representation and data augmentation, the highest accuracy among template-free methods was achieved. In this work, data augmentation was not employed, leaving room for potential improvements in accuracy for future endeavors.

FIG. 16D & FIG. 16E depict exemplary Model A and Model B workflows and performance. FIG. 16D shows that Model B has reaction center embedding and does not have center-labeled products in the output. FIG. 16E shows the USPTO-Full Top-K accuracy performance for previous models compared to the disclosed models. (¹If the correct reactants contain one of the 50 most commonly seen spectators in the USPTO-Full dataset, the reaction is removed from the test set. ²Reaction centers are provided. ³The maximum number of reaction centers is two) GLN [Hanjun Dai et al., arXiv:2001.01408 [cs, stat], January 2020], LocalRetro [Shuan Chen et al., JACS Au, October 2021], and Neuralsym [Marwin H. S. Segler, Chem. Eur. J., May 2017] in black are template-based selection methods. GraphRetro [Vignesh Ram Somnath et al., arXiv:2006.07038 [cs, stat], June 2021], RetroPrime [Xiaorui Wang et al., Chemical Engineering Journal, September 2021], and RetroExplainer [Yu Wang et al., Nat Commun, October 2023] in yellow are semi-template methods. GTA [Seung-Woo Seo et al., AAAI, May 2021], Tied-Transformer [Eunji Kim et al., J. Chem. Inf. Model., January 2021], MEGAN [Mikołaj Sacha et al., J. Chem. Inf. Model., July 2021], Transformer [Igor V. Tetko et al., Nat Commun, November 2020], and R-SMILES [Zipeng Zhong et al., Chem. Sci., 2022] in green are template-free generation methods. This work (in red) uses a template-generation method. Reactant-based selection methods are not included due to out-of-memory for the USPTO-FULL dataset [Zipeng Zhong et al., January 2023].

In addition, an analysis of the Top-K accuracy considering different numbers of reaction centers for Model B was conducted. Over half of the test reactions possess one or two reaction centers, following the same distribution of the reaction center counts of the training set. Consequently, for the test reactions with at most two reaction centers, Model B achieved the highest Top-K accuracy comparing to other center counts and the Top-10 accuracy reached 90% (see last row of FIG. 16E), showcasing exceptional predictive capabilities in scenarios characterized by a limited number of reaction centers. The high Top-K accuracy achieved by Model B for reactions with few reaction centers is particularly significant, as it corresponds to real-world applications where a majority of reactions feature a low number of reaction centers. For instance, 90% of the dataset comprises reactions with no more than four reaction centers.

Sampling Model with Latent Space

A sampling generative model, which exploits a sampling process with a latent space, is different from the deterministic approach. Prior to this disclosure, the application of a sampling model for retrosynthesis planning has not been explored. Model C is built upon the architecture of Conditional Kernel-Elastic Autoencoder (CKAE) [Haote Li et al., arXiv:2310.08685v1, October 2023]. Comparing to previous CKAE molecular generation models where conditions are represented by specific values or molecular properties, the CKAE model as applied to Model C utilizes the SMILES representation of target molecules as conditions.

In addition to the sampling feature, the encoder of Model C provides a valuable referencing feature. The encoder maps the input into a latent space with a distance regularized by the m-MMD loss [Haote Li et al., arXiv:2310.08685v1, October 2023]. This distance measure derived from the latent space provides a quantifiable metric to assess the similarity between reactions, aiding in evaluating and understanding the differences between chemical transformations. Such capability enables the identification and referencing of the most similar reactions within the dataset, facilitating comparison and analysis.

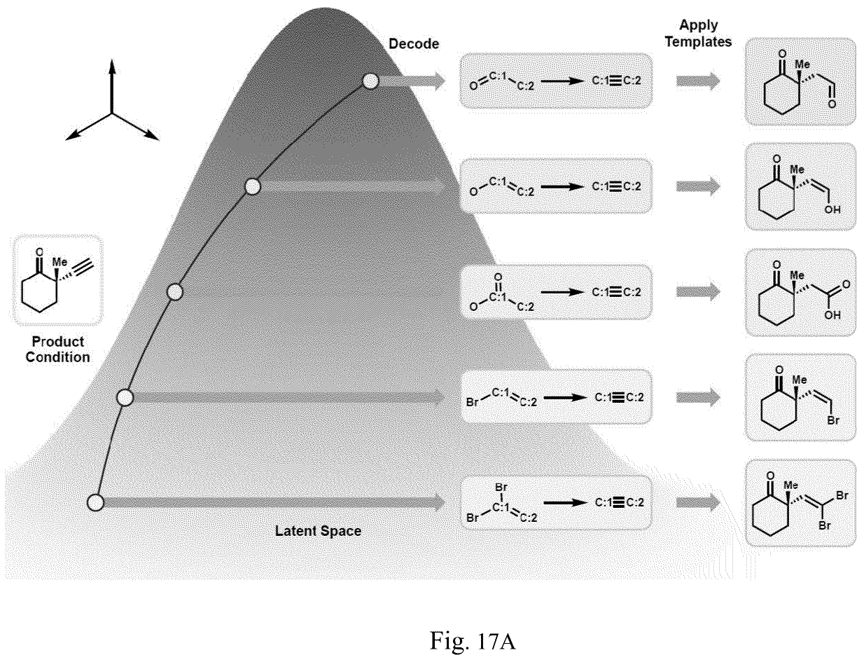

FIGS. 17A & FIG. 17B show interpolation of exemplary templates in the latent space of Model C and reactants from Model C outputs. FIG. 17A shows that the intermediates of the top and bottom latent representations are decoded. FIG. 17B shows the selected reactants for 2-, 3-, 4-substituted cyclohexanone derivatives as target compounds.

FIG. 17A illustrates the sampling workflow for Model C. During the sampling process, latent vectors are sampled and passed into Model C decoder after concatenating with target compound conditions. This generates SSTs and CLPs based on the given conditions.

In FIG. 17A, an interpolation process is visualized. Initially, two reaction templates were selected, represented by the top and bottom templates and the latent vectors in the latent space. These templates serve as the starting points to explore the intermediates. This interpolation allowed the discovery of the templates corresponding to each of the latent vectors along the path between the two originals. It can be observed that the middle templates and reactants form a blending of the starting templates and reactants. This observation provides evidence that the latent space captures chemical information, showing the distance measure between various chemical transformations.

To showcase the differences between Model A (deterministic) and Model C (sampling), both without reaction center information, the single-step predictions of 2-, 3- and 4-substituted cyclohexanone derivative is examined. Based on the acquired results, the representative precursors are chosen for all three target molecules (FIG. 17B). As it can be deduced from the figure, Model A suggestions are primarily based on functional group transformations and protection-deprotection reactions. On the other side, while Model C does suggest these transformations, diverse precursors/reactions are also proposed. These examples are not instinctively reached by humans and therefore, Model C could be utilized for inspiration in new approaches and method developments in synthetic chemistry.

### Experimental Validation

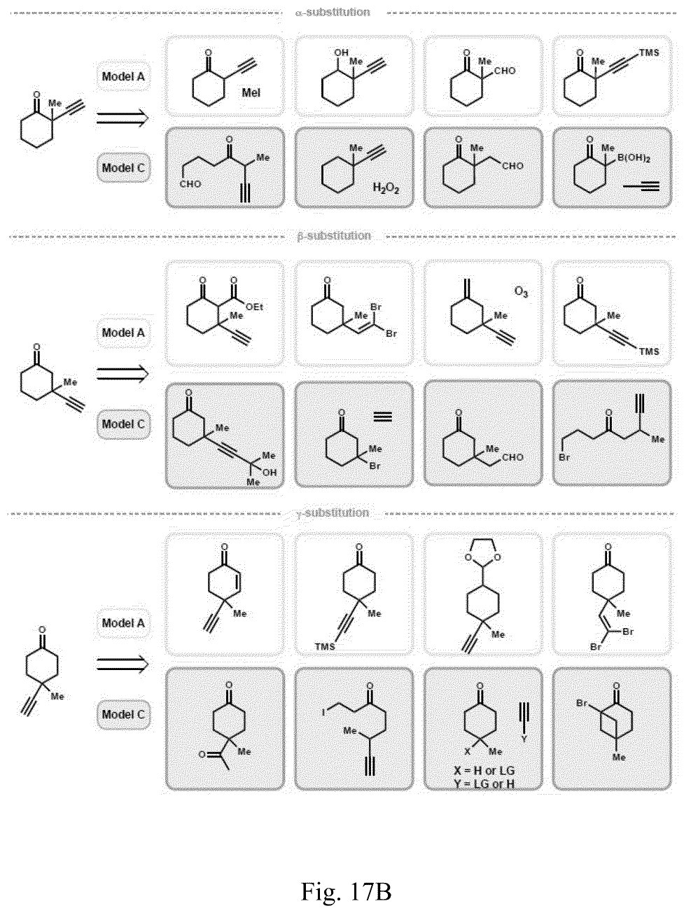

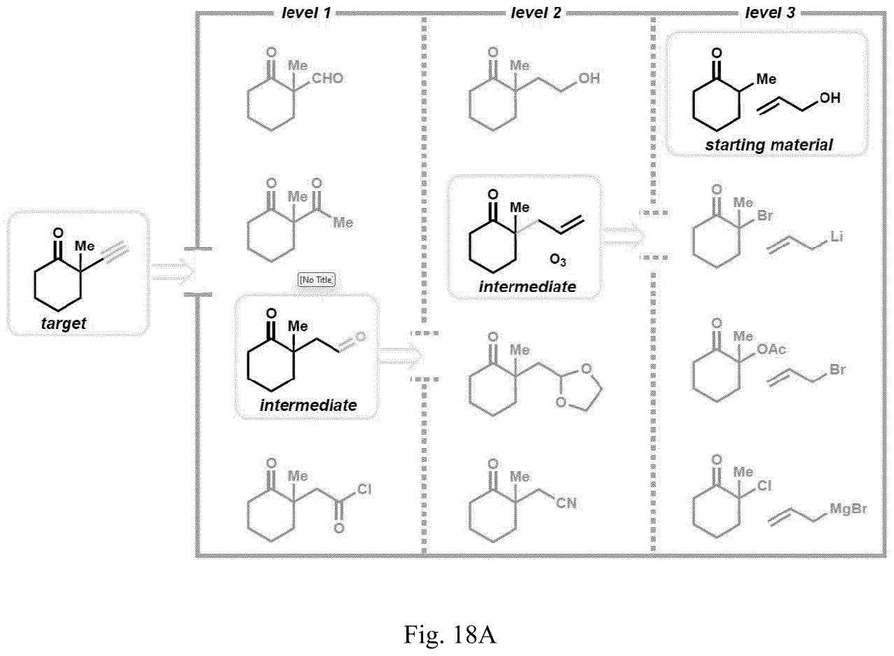

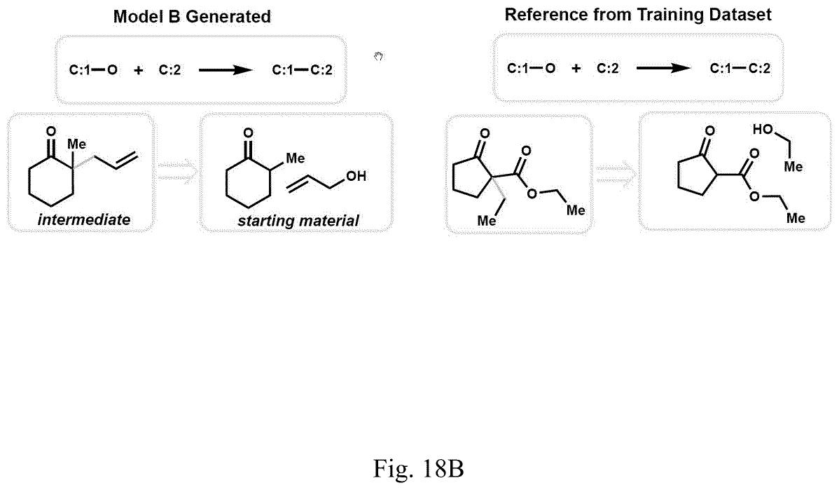

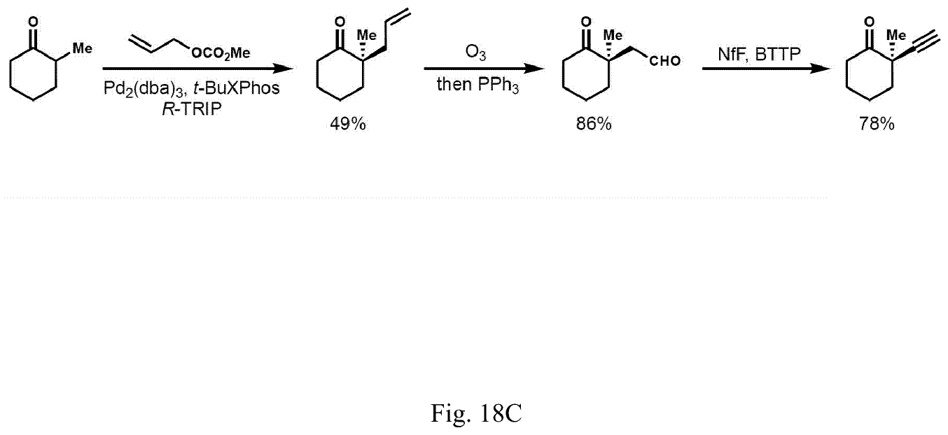

Developing cheap, fast and robust methods for the synthesis of bioactive molecules and their precursors is one of the key goals in the pharmaceutical chemistry [M. D. Eastgate et al., Nature Reviews Chemistry, 2017]. The disclosed Model B was chosen because of its high accuracy and reaction center embedding, for establishing the shortest route for the synthesis of target compound (see FIG. 18A). Previously synthesized in 5 steps [Jason ABBOTT et al., U.S. Patent 2023/0212164 A1, 2023], target molecule can now be accessed in 3 steps by hand picking the reactants that the model suggested for each retrosynthetic step. Following chemical intuition for choosing the molecules, the aldehyde-group was chosen as a precursor for the alkyne moiety. The carbonyl functional group can be acquired by ozonolysis of the corresponding alkene, which can be introduced via allylation reaction.

FIG. 18A through 18C shows an exemplary retrosynthesis tree for compound 1 and its experimental procedure. FIG. 18A shows that a synthesis route is selected from the retrosynthesis tree generated by Model B. FIG. 18C shows an exemplary reference found with Model C for the allylation step. FIG. 18B shows the related experimental procedure of the selected route.

FIG. 18B serves as a reference point derived from Model C. The left-hand side illustrates the allylation step employed in the disclosed synthesis. On the right-hand side, the reference is obtained by encoding the allylation template and the product labeled with the reaction center into Model C's latent space. This process allows one to identify the closest latent vectors from the training dataset, and that closest reference corresponds to the reaction shown on the right-hand side of FIG. 18B.

In order to synthesize enantiomerically pure target molecule, it was chosen to introduce the chiral center and the allylic moiety simultaneously, by applying the enantioselective Pd-catalyzed method reported by Pupo et al. [G. Pupo et al., Angewandte Chemie-International Edition, 2016]. The corresponding product was treated with ozone in order to obtain the ketoaldehyde derivative in good yield (see FIG. 18C). For the final step, a modified procedure by Boltukhina et al. [E. V. Boltukhina et al., Tetrahedron, 2011] was applied and the target product was isolated in 78 percent yield. The described experimental procedure demonstrates that the newly developed machine learning model can significantly aid in development of synthetic routes for pharmaceutically important molecules as well as improve the already reported ones.

An alternative to the route presented in FIG. 18C, an even shorter route to compound 1, could be one entailing direct α-alkynylation of 2-methylcyclohexanone. Methods for direct introduction of alkyne moiety next to ketone are scarce and rely on substitution with electrophilic alkyne species (Selected examples: [A. S. Kende et al., Tetrahedron Letters, 1982; Y. Nishimura et al., Tetrahedron Letters, 2006; A. Utaka et al., Chemical Communications, 2014; M. Wegener et al., Organic Letters, 2015; J. Wang et al., Journal of the American Chemical Society, 2020; D. Jang et al., Angewandte Chemie-International Edition, 2021]). Most commonly used in modern organic chemistry are hypervalent iodine reagents such as Waser's or Ochiai's reagent [D. P. Hari et al., Accounts of Chemical Research, 2018]. While this method would furnish the target molecule in smaller number of synthetic steps, it would have to be followed by separation of two enantiomers since enantioselective α-alkynylation of ketones has not yet been reported.

In this work, a string-based approach for retrosynthesis planning that leverages generative models to address the challenges posed by the vast chemical space and synthesis complexity is introduced. Notably, this work introduces a novel category in ML methods for CASP: template generation. The disclosed work encompassed the development and evaluation of two types of generative models: deterministic generative models (S2S) and a sampling generative model that utilizes CKAE.

Model A and Model B are benchmarked on the USPTOFULL dataset. Notably, Model B can incorporate reaction centers using positional embeddings, enabling the generation of SSTs that apply to the reacting sites. On the other hand, Model C represents a pioneering application of sampling method from latent space in CASP, capable of generating diverse reactions. The design of Model C defines distances between reactions, which allows Model C to identify the closest reference from the dataset for newly generated templates, making it a suitable tool for generating and validating a wide range of potential reactions.

This work presents two approaches for single-step synthetic planning: high-accuracy deterministic models and high-diversity sampling models. The capability of specifying reacting sites, the availability of relevant reaction references, and the successful results of experimental validations make the three models valuable tools in guiding retrosynthetic analysis.

### Methods—Training Details

10% dropout was applied to all attention matrices and embedding vectors. During training, each token in the input to the encoders has a 15% chance of being replaced by a mask token. ADAM optimizer [Diederik P Kingma et al., Adam: A method for stochastic optimization, 2014] was used with a learning rate of 5×10−5. Gradient normalization [Zhao Chen et al., International conference on machine learning, 2018] was set to 1.0.

### Model Architecture

Model A, B, and C each has six layers of Transformer encoders and decoders. For Model A and B, an embedding size of 256 was used and for Model C, an embedding size of 512 was used.

### Beam Search

To derive multiple possible predictions, beam search [Igor V. Tetko et al., Nat Commun, November 2020] is used across all models. During decoding, the transformer decoder attends to the encoder output and the sequence that had been generated. The decoder outputs probabilities of all possible tokens for the next position in the sequence. Beam search maintains a fixed-size set of candidate sequences, the number that the method keeps is called the beam size B. The top B most probable sequences at each decoding step are selected to proceed to the next step of decoding until the stopping criteria of maximum allowed length is reached or an End Of Sequence (<EOS>) token is output.

For the Top-K accuracy test, beam search with a beam size of 50 was used during all decoding processes. At each decoding step, the model outputs the 50 most probable candidate tokens and continues the sequence until the stopping criteria is met.

The diversity of deterministic models is solely derived from the beam search process, as this type of model lacks a latent space for sampling. Consequently, generating novel reactions using a deterministic model through beam search can be challenging. In contrast, the sampling model, equipped with a latent space, can generate diverse and novel reactions more effectively.

The 50 most commonly seen spectators are obtained from the USPTO-Full reaction file on RDChiral GitHub Repository [Connor W. Coley et al., J. Chem. Inf. Model., June 2019]. While the train-validation-test split of the USPTO-Full dataset is obtained from the GitHub repository of [Igor V. Tetko et al., Nat Commun, November 2020].

### Reaction Template: RDChiral Template Vs Site-Specific Template

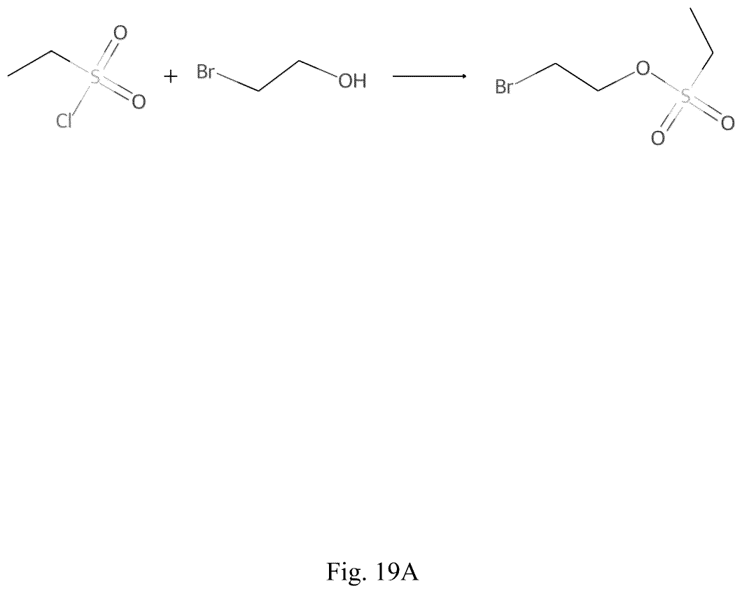

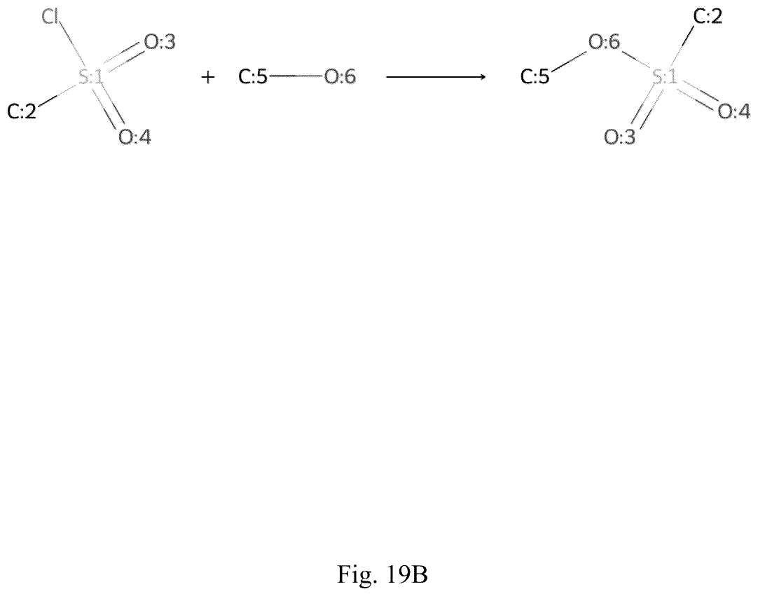

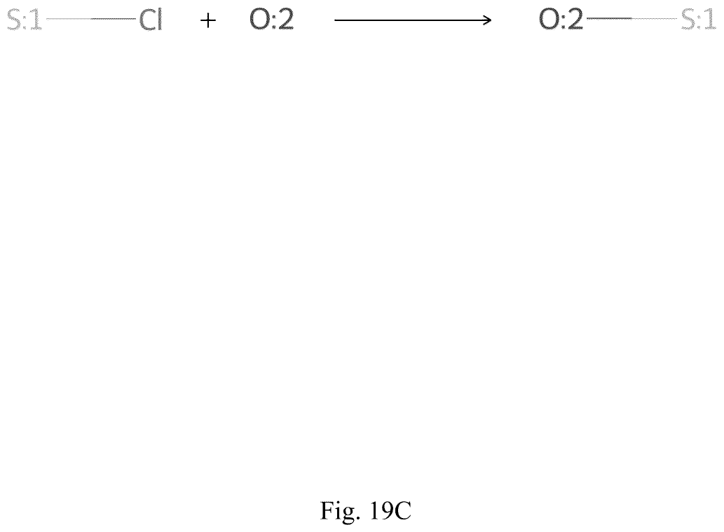

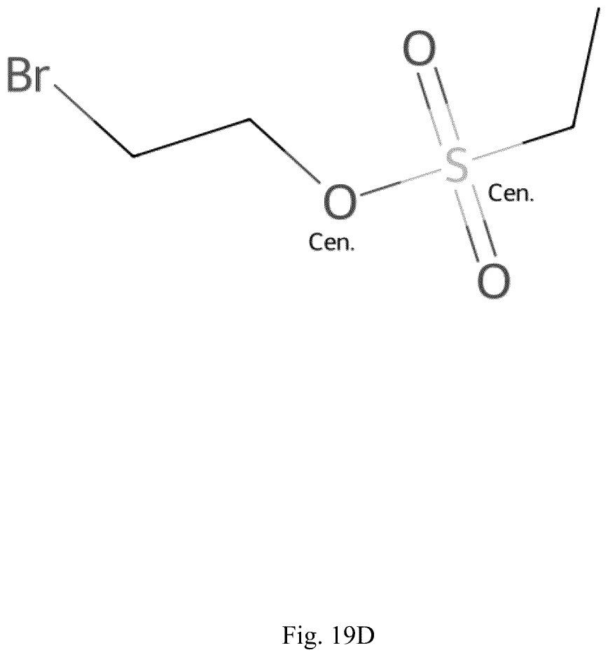

FIGS. 19A through 19D show exemplary reaction templates showing RDChiral Template vs Site-Specific Template. FIG. 19A shows an exemplary Reaction Example. FIG. 19B shows an exemplary RDChiral Template. FIG. 19C shows an exemplary Site-Specific Template. FIG. 19D shows an exemplary Center-Labeled Product. Shown in FIGS. 19A through 19D are a visualization for the reaction SMARTS string in (FIG. 19A): CCS(═O)(═O)Cl.OCCBr>>CCS(═O)(═O)OCCBr. Using the RDChiral template in (b): [C:5]−[O;H0;D2;+0:6]−[S;H;D4;+0:1](−[C:2])(=[O;D1;H0:3])=[O;D1;H0:4]>>Cl−[S;H0;D4;+0:1](−[C:2])(=[O;D1;H0:3])=[O;D1;H0:4].[C:5]−[OH;D1;+0:6] and the product/target compound: CCS(═O)(═O)OCCBr, the reaction SMARTS string in (FIG. 19A) can be obtained. Alternatively, in this work, the reaction SMARTS string can be obtained from the site-specific template in (FIG. 19C): [O:2]−[S:1]>>Cl−[S:1].[OH:2] and target compound with reaction centers labeled in (FIG. 19D): CC*(═O)(═O)*CCBr.

A reaction template is a concise representation of a chemical reaction, capturing the essential information about the substructure changes occurring during the reaction. In the context of retrosynthesis, reaction templates provide a valuable tool for generating potential pathways to synthesize target molecules. The format of reaction templates is typically represented as PRODUCT>>REACTANT in the retro-direction, indicating the transformation from the product back to the reactant. However, for the purpose of visualization in the disclosed work, the forward-direction format was adopted since it is more intuitive for understanding the reaction process. FIGS. 19A through 19D illustrate an example reaction template visualization for the reaction SMARTS string in FIG. 19A.

Previous template-based methods have commonly utilized template extraction codes from the RDChiral repository to extract reaction templates. These templates include not only the reaction centers but also neighboring atoms and special functional groups, providing a comprehensive representation of the chemical transformations as demonstrated in FIG. 19B. However, in the disclosed work, the template extraction process was modified to focus exclusively on the reaction centers as depicted in FIG. 19C. These modified templates are referred to herein as site-specific templates since they specifically apply to the reacting sites (reaction centers) of the target compounds. To incorporate this specificity, additional input was introduced in the form of reaction center labels. These labels indicate the specific sites within the target compound where the template should be applied. FIG. 19D showcases an example of a reaction center-labeled target molecule.

Specificity from Reaction Center-Labeled Products

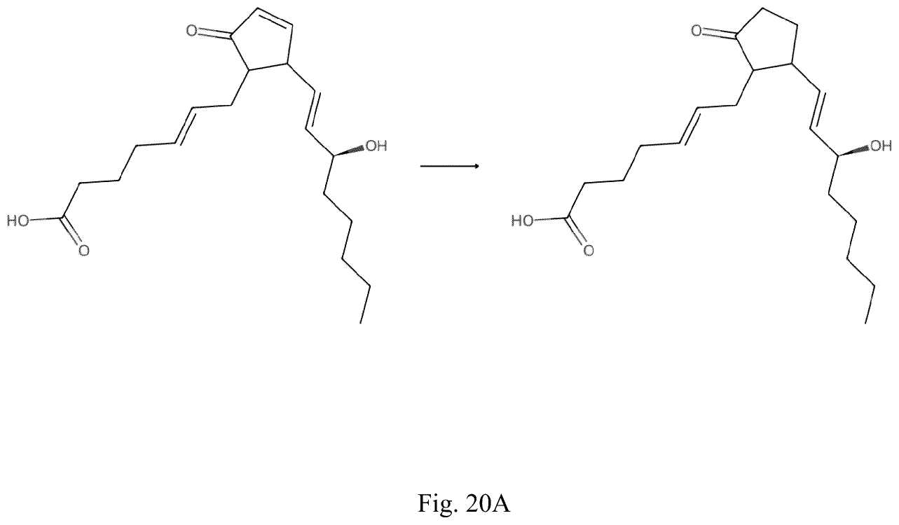

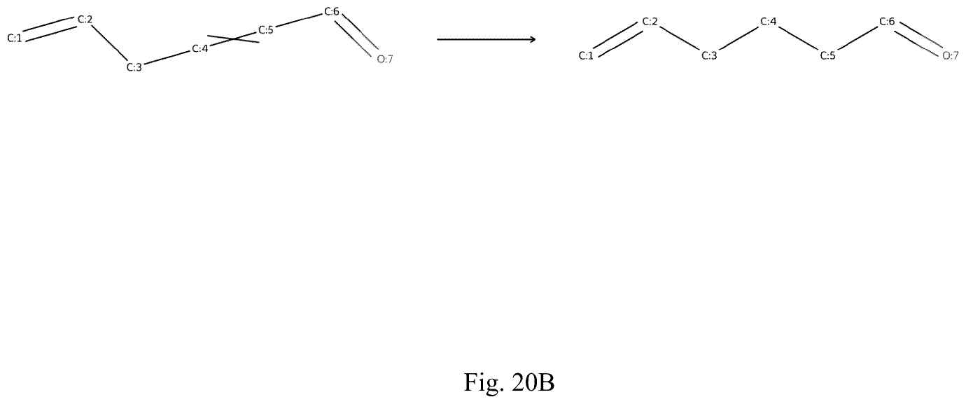

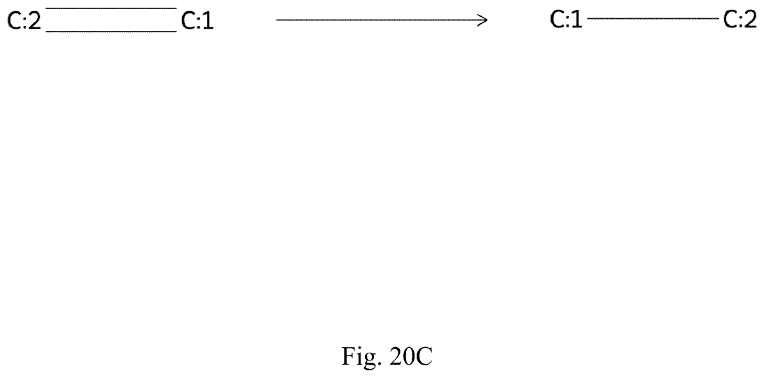

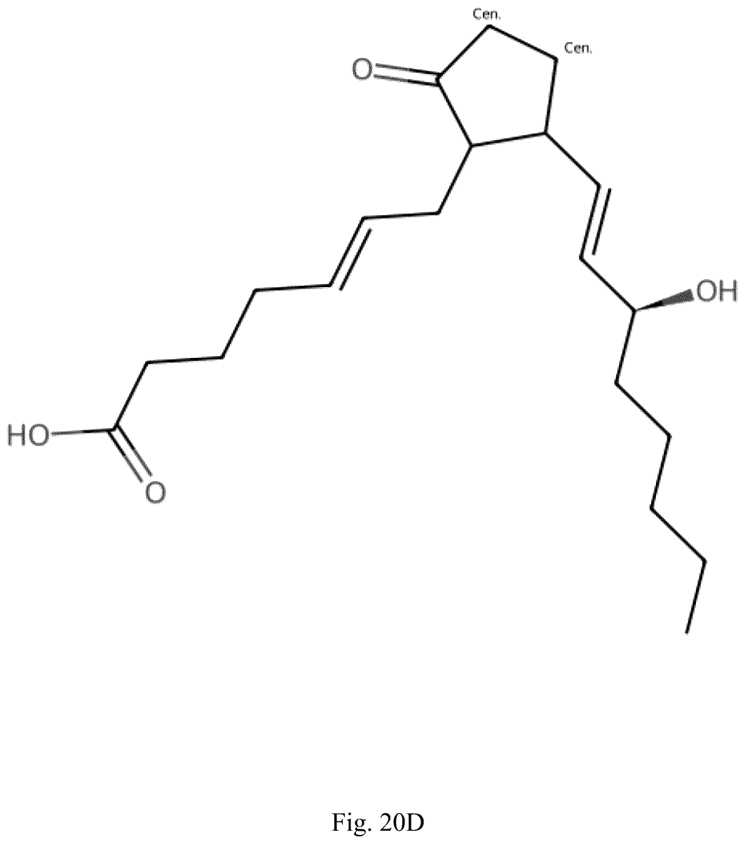

An important aspect of the disclosed site-specific template approach is that the specificity is given by reaction center-labeled products. While the site-specific templates focus exclusively on the reaction centers, they lack the necessary information to determine the precise locations/atoms within the target compound where the template should be applied. FIGS. 20A through 20D provide an illustrative example of how the reaction center-labeled target compound plays a crucial role in achieving specificity.

In FIG. 20A, a specific chemical reaction is presented involving a carbon-carbon double bond reduction. The RDChiral template (see FIG. 20B) offers a comprehensive representation of the transformation, including the reaction centers, neighboring atoms, and special functional groups. It is evident from the RDChiral template that the carbon-carbon double bond reduction occurs at a specific location within the molecule. However, when the site-specific template (FIG. 20C) is considered, which solely captures the reaction centers, a lack of specificity is observed. Multiple carbon pairs in the product can potentially undergo the same transformation, resulting in ambiguity.

FIG. 20A through 20D show an exemplary reaction wherein a site-specific template requires a product/target compound with reaction centers labeled in order to get the reaction smart string: CCCCC[C@H](O)C═CC1C═CC(═O)C1CC═CCCCC(═O)O>>CCCCC[C@H](O)C═CC1CCC(═O)C1CC═CCCCC(═O). FIG. 20A shows an exemplary Reaction Example. FIG. 20B shows an exemplary RDChiral Template. FIG. 20C shows an exemplary Site-Specific Template. FIG. 20D shows a resultant Center-Labeled Product.

To resolve this ambiguity and introduce specificity, the reaction center-labeled target compound (see FIG. 20D) was utilized. By labeling the specific reaction centers within the product molecule, the precise locations were indicated where the site-specific template should be applied. In this example, the labeled reaction centers specify the carbon-carbon double bond that needs to be reduced. By combining the site-specific template and the labeled product molecule, the accurate reaction SMARTS string was obtained that represents the desired chemical transformation.

### Template Generation Deterministic Model Architecture

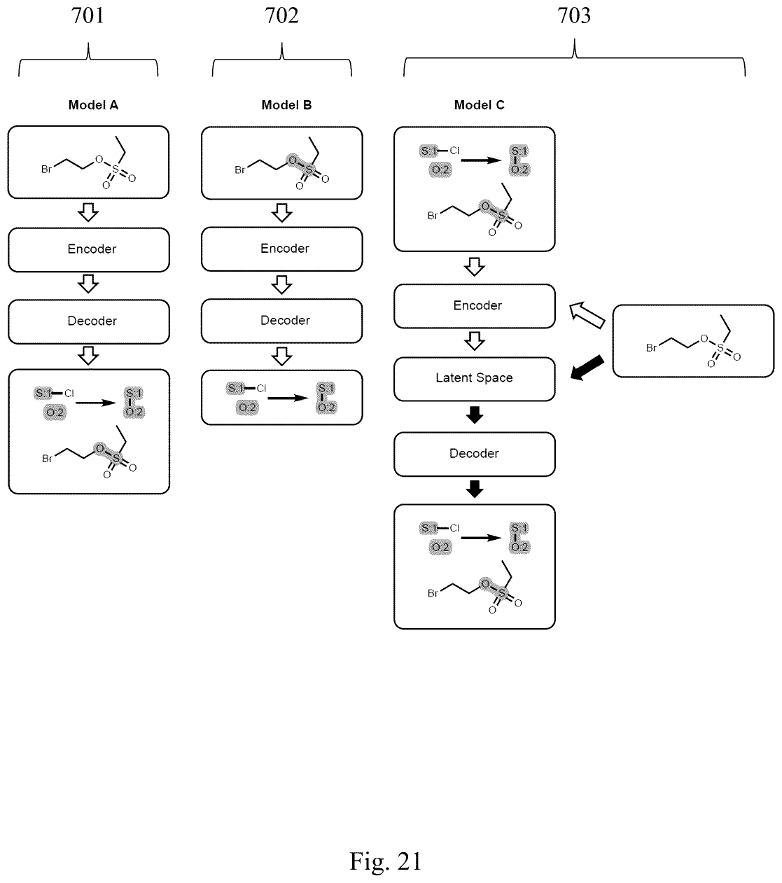

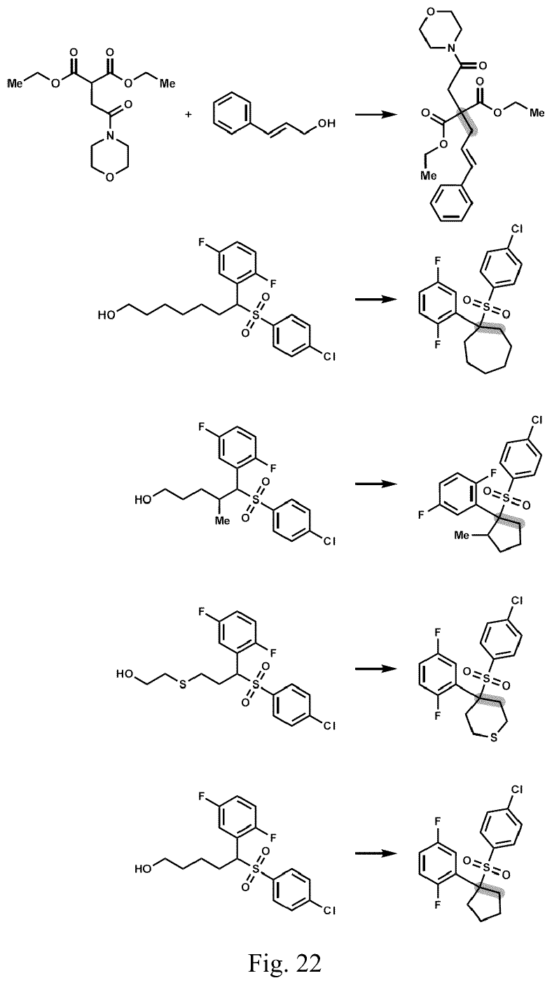

FIG. 21 shows exemplary model architectures of the generative models for retrosynthesis planning, comprising columns 701, 702 and 702. Column 701 comprises Model A, which is a deterministic generative model that takes in target products and output site-specific templates and labeled products. Column 702 comprises Model B, a variant of Model A, incorporating positional embeddings for conditioning on specific reacting sites. Column 703 comprises Model C, a sampling generative model based on the conditional kernel-elastic autoencoder (CKAE) approach.

Referring now to FIG. 21, Column 701 illustrates the model architecture of the disclosed deterministic approach (Model A). The model employs a transformer encoder to capture the relevant features and representations of the target molecule. Subsequently, these encoded features are fed into a transformer decoder, which generates the site-specific template and the product with reaction centers labeled.

In the disclosed example, referring back to FIGS. 19A through 19D, we consider the example reaction in FIG. 19A, the site-specific template in FIG. 19C, and the product with reaction centers labeled in FIG. 19D. The input of the deterministic model consists of the product molecule obtained from the reaction, such as CCS(═O)(═O)OCCBr in the disclosed example. The output of the deterministic model is structured in the following format: [0:2]−[S:1]>>Cl−[S:1].[OH:2]_CC*(═O)(═O)*CCBr. Here, the site-specific template is represented by [0:2]−[S:1]>>Cl−[S:1], indicating the breaking of the S—Cl bond and the formation of an S—O bond. The product with reaction centers labeled, CC*(═O)(═O)*CCBr, highlights the third and sixth atoms as the reaction centers using asterisks. With the generated site-specific template and the labeled product, one can reconstruct the original reaction depicted in FIG. 19A.

In addition, the disclosed deterministic generative model offers the flexibility to control the exact atoms participating in reactions by incorporating the relevant information within the encoder. This variation, denoted as Model B in Column 701, introduces a fixed embedding for the “*” token, representing the positions of the reacting atoms. Such positional information and the product SMILES input are passed in as model input. The output of Model B consists solely of site-specific templates, as the reaction centers are explicitly provided. This variant model allows researchers to customize the reaction centers by specifying the atoms involved. Such unique feature allows for precise control over retrosynthetic disconnections/transformations.

### Template Generation Sampling Model Architecture

Referring again to FIG. 21, Column 701 illustrates the model architecture of the disclosed sampling approach (Model C). Using the example in FIGS. 19A through 19D again, by incorporating the product CCS(═O)(═O)OCCBr as the condition, the transformer encoder processes the site-specific template and the labeled product ([O:2]−[S:1]>>Cl−[S:1].[OH:2]_CC*(═O)(═O)*CCBr) at the same time and passes it through the latent space. The decoder is then tasked with reproducing the same input ([O:2]—[S:1]>>Cl—[S:1].[OH:2]_CC*(═O)(═O)*CCBr) as the output. This comprehensive encoding and decoding process where site-specific templates and labeled products are processed at the same time enables an attention model to capture essential information for single-step prediction, including the influence of functional groups on reactivity and regioselectivity. During the sampling phase (shown as shaded arrows in Column 703), given target products as conditions and random latent vectors, the model can generate a variety of templates and center-labeled products, leveraging the flexibility of the latent space and the conditioning on target molecules.

CKAE incorporates a specially designed loss function known as modified Maximum Mean Discrepancy (m-MMD), which enhances the generative power of the model. CKAE also utilizes a weighted cross-entropy loss, with the weights controlled by the 6 and X parameters, to improve the reconstruction capability. Additionally, CKAE presents exceptional correlations between outputs and given conditions. Further details on these loss functions and correlation results can be found in the CKAE paper [Haote Li et al., arXiv:2310.08685v1, October 2023].

While both deterministic and sampling models aim to accurately predict templates and center-labeled products, the sampling model offers additional capabilities. By incorporating a latent space and conditioning on target molecules, the sampling model has the ability to generate diverse and novel reactions. Leveraging the latent space, the model can sample reactions beyond the provided templates, resulting in a broader range of potential transformations. In contrast, deterministic models lack a latent space, limiting their ability to extrapolate and generate innovative reactions. The CKAE paper [Haote Li et al., arXiv:2310.08685v1, October 2023] showcases the superior interpolation and extrapolation capabilities of the sampling model, highlighting its capacity to sample a wider range of diverse reactions.

### Other References for the Allylation Step

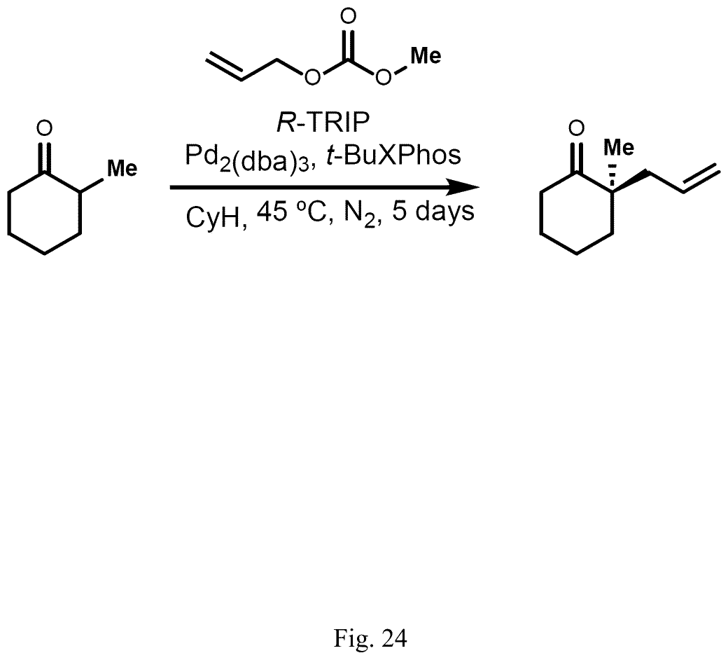

FIG. 24 presents a compilation of the top 10 references for the allylation step depicted in FIG. 18B. The site-specific templates are the same for these 10 references. Therefore, the products of these reactions are the primary determinant for the ranking (latent distance) in this particular case.

### Encoder-Decoder Attention for Site-Specific Templates and Center-Labeled Product

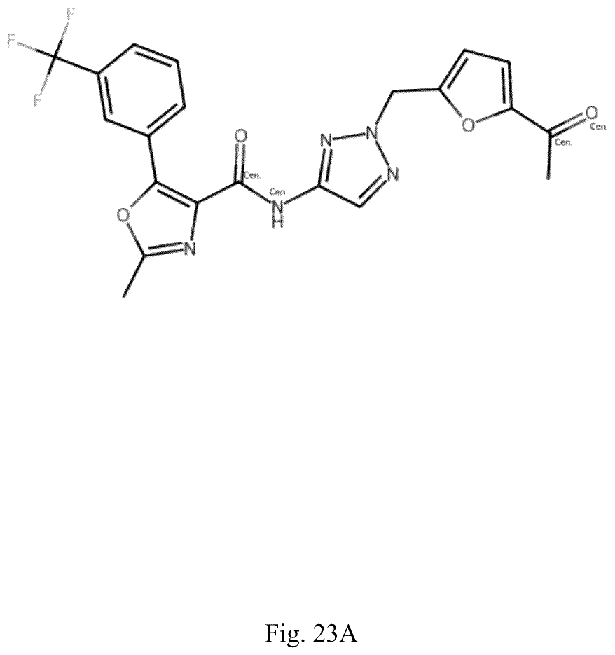

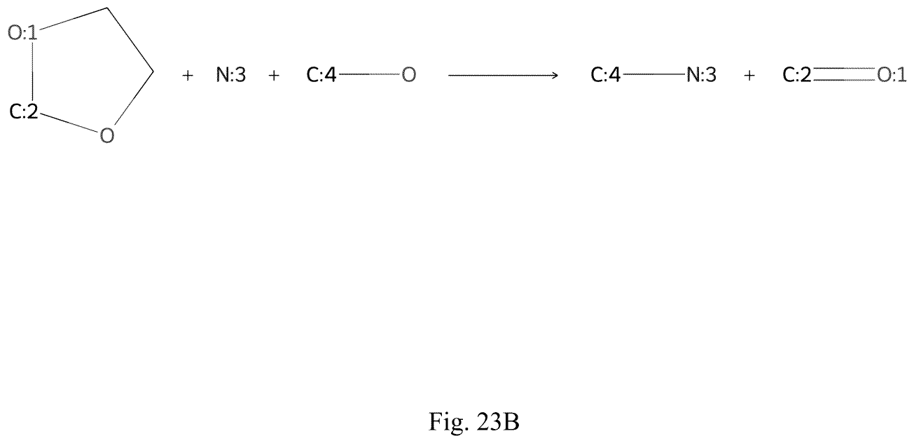

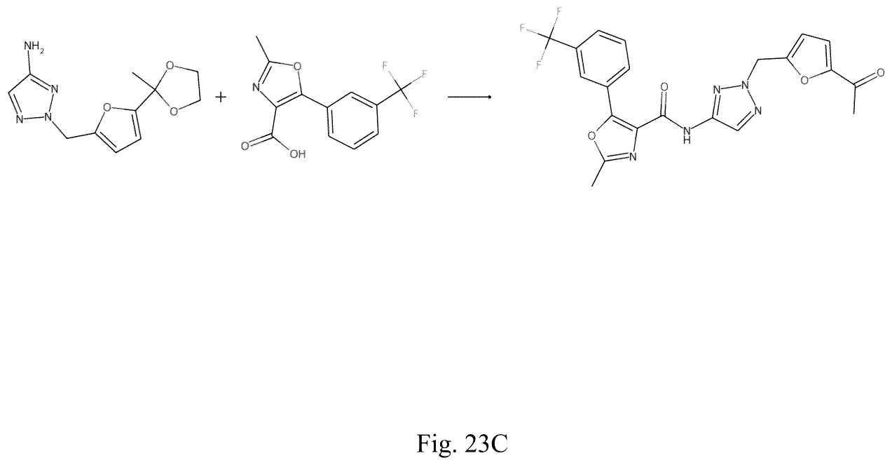

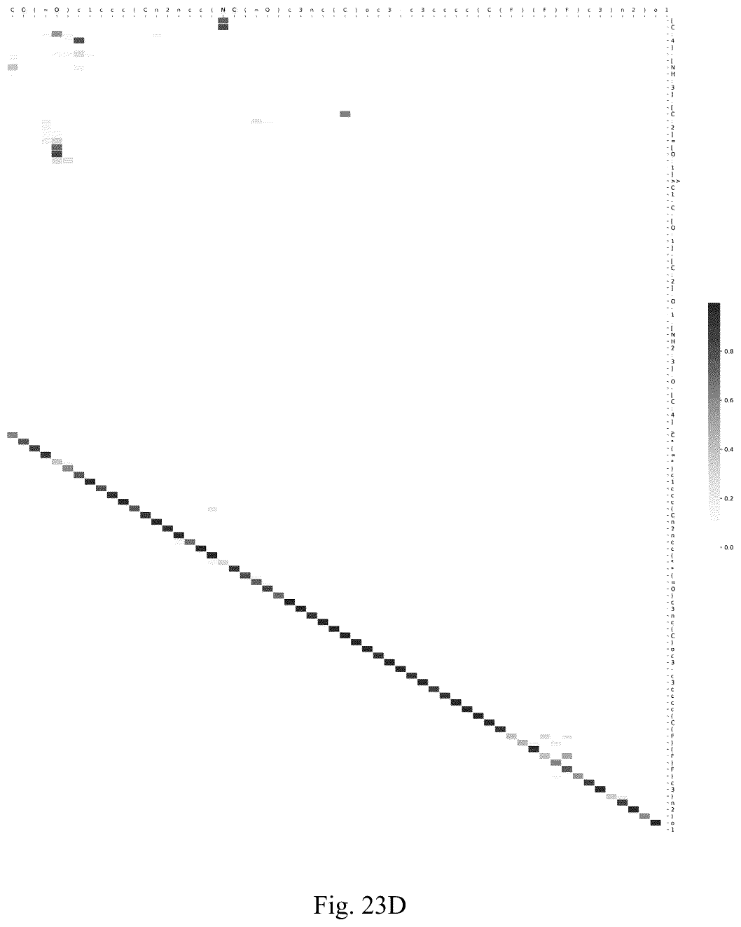

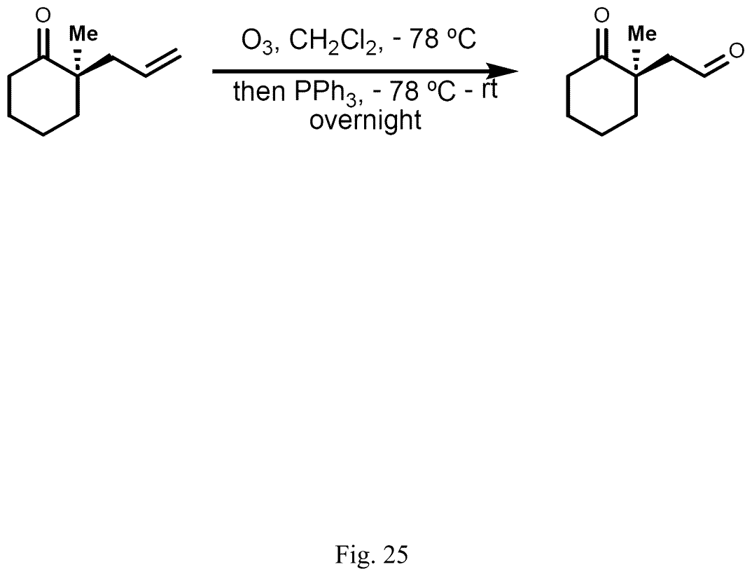

Herein is a demonstration of the disclosed model's attention mechanism as shown in FIGS. 25A through 25D, highlighting its ability to capture essential chemical information like functional groups and regioselectivity during the generation of reaction templates. This is illustrated through an example using the disclosed deterministic model, without the inclusion of reaction center information from positional embedding (Model A). The input of the model is the product in FIG. 23A (without the labels of reaction centers). The output of the model is the template shown in FIG. 23B along with the labeled product where the reaction centers are labeled in FIG. 23A. The corresponding reaction is shown in FIG. 23C where it is an amide bond formation and a removal of protection group for the ketone.

In FIG. 23D, the encoder-decoder attention matrix is shown, where the column labels on top represent the encoder input product SMILES, and the row labels on the right represent the decoder output template and labeled product. The reaction centers from the row labels are highlighted in yellow for encoder input for better visualization (the column labels). The presence of the ketone oxygen, originating from the protection group removal, significantly affects the output. Also, the matrix reveals that the influence on the template output extends beyond the reaction centers. Furthermore, the product input affects the labeled product portion of the output, resulting in a distinct diagonal pattern in the bottom of the matrix. These findings demonstrate the model's integration of critical chemical features that enhance its ability to generate accurate and relevant reaction templates.

FIGS. 23A through 23D show a visualization of the encoder-decoder-attention obtained from the product: CC(═O)c1ccc(Cn2ncc(NC(═O)c3nc(C)oc3-c3cccc(C(F)(F)F)c3)n2)o1. FIG. 23A shows the Encoder Input Product (centers are from decoder output). FIG. 23B shows the Decoder Output Template. FIG. 23C shows the Corresponding Reaction. FIG. 23D shows the encoder-Decoder Attention Matrix.

TABLE 7
USPTO Full Top-K accuracy (in %) comparison.

| Method^{a} | Model | Top-1 | Top-3 | Top-5 | Top-10 | Top-20 | Top-50 |
|---|---|---|---|---|---|---|---|
| Template-Based | GLN[26] | 39.3 | | | 63.7 | | |
| | LocalRetro[27]^{b} | 39.1 | 53.3 | 58.4 | 63.7 | 67.5 | 70.7 |
| | Neuralsym[22]^{b} | 42.7 | 58.7 | 63.4 | 67.9 | 70.8 | 72.1 |
| Semi-Template | GraphRetro[32]^{b} | 24.8 | 34.5 | 36.9 | 38.7 | 39.5 | 39.8 |
| | RetroPrime[33]^{b} | 45.8 | 61.6 | 63.9 | 70.3 | 71.2 | 72.6 |
| | RetroExplainer[34] | 51.4 | 70.7 | 74.7 | 79.2 | | |
| Template-Free | GTA[43]^{b} | 46.6 | 52.5 | 57.9 | 63.3 | 67.2 | 70.4 |
| | Tied- | 37.7 | 53.6 | 58.7 | 63.7 | 67.8 | 71.0 |
| | Transformer[48]^{b} | | | | | | |
| | MEGAN[45] | 33.6 | | | 63.9 | | 74.1 |
| | Transformer[42]^{b} | 44.7 | 61.1 | 66.0 | 70.7 | 74.1 | 76.2 |
| | R-SMILES[50] | 48.9 | 66.6 | 72.0 | 76.4 | 80.4 | 83.1 |
| Template- | Model A | 34.4 | 52.2 | 58.3 | 64.5 | 69.2 | 72.6 |
| Generation | Model A^{c} | 37.3 | 56.2 | 62.6 | 68.8 | 73.3 | 76.6 |
| (This Work) | Model B^{d} | 48.1 | 67.8 | 72.6 | 76.4 | 78.7 | 80.2 |
| | Model B^{c,d} | 51.1 | 71.6 | 76.4 | 80.0 | 82.0 | 83.3 |

^{a}Reactant-based methods are not included due to out-of-memory for USPTO-Full dataset.
^{b}Results obtained from [Shuan Chen et al., JACS Au, October 2021].
^{c}If the correct reactants contain one of the 50 most commonly seen spectators in the USPTO Full dataset, the reaction is removed from the test set.
^{d} Positional embedding of the reaction centers are included.

Hanjun Dai et al., arXiv:2001.01408 [cs, stat], January 2020; [19] Shuan Chen et al., JACS Au, October 2021; [14] Marwin H. S. Segler, Chem. Eur. J., May 2017; [24] Vignesh Ram Somnath et al., arXiv:2006.07038 [cs, stat], June 2021; [25] Xiaorui Wang et al., Chemical Engineering Journal, September 2021; [26] Yu Wang et al., Nat Commun, October 2023; [34]Seung-Woo Seo et al., AAAI, May 2021; [39] Eunji Kim et al., J. Chem. Inf. Model., January 2021; [36] Mikołaj Sacha et al., J. Chem. Inf. Model., July 2021; [33] Igor V. Tetko et al., Nat Commun, November 2020; [31] Kangjie Lin et al., Chem. Sci., 2020; [11] Zipeng Zhong et al., January 2023.

TABLE 8
Top-K accuracy (in %) for different number
of reaction centers using Model B.

| Maximum | | | | | | | % of |
|---|---|---|---|---|---|---|---|
| Reaction | Top- | Top- | Top- | Top- | Top- | Top- | Test |
| Centers | 1 | 3 | 5 | 10 | 20 | 50 | Data^{a} |
| No Limit^{b} | 51.1 | 71.6 | 76.4 | 80.0 | 82.0 | 83.3 | 90.7% |
| 5 | 53.0 | 74.1 | 79.0 | 82.6 | 84.6 | 86.0 | 85.2% |
| 4 | 54.7 | 76.0 | 80.9 | 84.5 | 86.5 | 87.8 | 81.5% |
| 3 | 57.8 | 78.9 | 83.8 | 87.3 | 89.2 | 90.4 | 75.1% |
| 2 | 61.2 | 81.7 | 86.6 | 89.9 | 91.7 | 92.8 | 58.3% |
| 1 | 60.1 | 80.8 | 86.3 | 90.3 | 92.7 | 93.5 | 11.6% |

^{a}Reactions containing 50 most common spectators as reactants are removed for all these cases, so no limit does not mean 100% of the test data.
^{b}The maximum reaction center count in test set is 18, while the maximum for training set is 19.

### Experimental Section—General

All reactions were carried out under an inert nitrogen atmosphere with dry solvents under anhydrous conditions unless otherwise stated. Stainless steel cannula or syringe was used to transfer solvent, and air- and moisture sensitive liquid reagents. Reactions were monitored by thin-layer chromatography (TLC) carried out on 0.25 mm Merck silica gel plates (60F254) using UV light as the visualizing agent and potassium permanganate and an acidic solution of p-anisaldehyde, on SiO₂ as developing agents. Flash column chromatography employed SiliaFlash® P60 (40-60 m, 230-400 mesh) silica gel purchased from SiliCycle, Inc.

Materials: Pd₂(dba)₃ was purchased from Strem. t-BuXPhos was purchased from Sigma Aldrich. R-TRIP ((R)-3,3′-bis(2,4,6-triisopropylphenyl)-1,1′-binaphthyl-2,2′-diyl hydrogenphosphate) was purchased from AmBeed. NfF (nonafluorobutanesulfonyl fluoride) was purchased from Oakwood Products, Inc. BTTP (tert-butyliminotri(pyrrolidino)phosphorane) was purchased from Sigma Aldrich. Dry cyclohexane and DMF were purchased from Sigma Aldrich. All other reagents were used as received without further purification, unless otherwise stated.

Instrumentation: All new compounds were characterized by means of ¹H NMR, ¹³C NMR, FT-IR, and HR-MS. Optical rotations were measured on Polarimeter Rudolph Autopol IV at 589 nm, 22° C. Data are reported as: [α]D^{t}, concentration (c in g/100 mL) and solvent. The absolute configurations were determined by comparison between the measured optical rotations and the reported values in literature. Copies of the ¹H- and ¹³C-NMR spectra can be found after experimental procedures. NMR spectra were recorded using a Varian 400 MHz NMR spectrometer. All ¹H NMR data are reported in units, parts per million (ppm), and were calibrated relative to the signals for residual chloroform (7.26 ppm) in deuterochloroform (CDCl₃). All ¹³C NMR data are reported in ppm relative to CDCl₃ (77.2 ppm) and were obtained with ¹H decoupling unless otherwise stated. The following abbreviations or combinations thereof were used to explain the multiplicities: s=singlet, d=doublet, t=triplet, q=quartet, m=multiplet. All IR spectra were taken on an FT-IR Shimadzu IRTracer-100 (thin film. High resolution mass spectra (HRMS) were recorded on a LC-MS Shimadzu 9030 Quadrupole Time-of-Flight high resolution mass spectrometer.

### Synthesis

FIG. 24 depicts the synthesis of (R)-2-Allyl-2-methylcyclohexan-1-one [D. C. Behenna et al., Journal of the American Chemical Society, 2004]. For the synthesis of (R)-2-allyl-2-methylcyclohexan-1-one, procedure reported by Pupo et. al was applied [G. Pupo et al., Angewandte Chemie-International Edition, 2016]. To a flame-dried microwave vial equipped with a magnetic stir bar were added Pd2(dba)3 (75.4 mg, 0.0824 mmol, 5 mol % Pd), t-BuXPhos (154 mg, 0.362 mmol, 11 mol %), R-TRIP (248 mg, 0.329 mmol, 10 mol %), 3 Å molecular sieves (3.3 g), cyclohexane (33 mL), 2-methylcyclohexanone (400 μL, 3.29 mmol, 1 equiv) after which allyl methyl carbonate (1.12 mL, 9.88 mmol, 3 equiv) was added dropwise. The reaction vial was capped and placed into a pre-heated 45° C. oil bath and stirred for 5 days. The reaction mixture was removed from the oil bath and cooled to ambient temperature before filtering through a short pad of celite. The celite was washed with Et₂O (30 mL) and the solution was concentrated under reduced pressure by rotary evaporation. Purification by flash column chromatography on silica gel (Et₂O/pentane=1:99 to 5:95) afforded the product (245 mg, 49%) as a colorless oil. R_{f}: 0.50 (EtOAc/Hex=1:9); [α]D²²: 40.96 (c=0.166, CH₂Cl₂); ¹H NMR (400 MHz, CDCl₃): 5.75-5.63 (m, 1H), 5.10-4.98 (m, 2H), 2.43-2.31 (m, 3H), 2.27-2.18 (m, 1H), 1.91-1.65 (m, 5H), 1.63-1.54 (m, 1H), 1.07 (s, 3H); ¹³C NMR (100 MHz, CDCl₃): 215.5, 133.9, 118.0, 48.6, 42.1, 38.9, 38.7, 27.5, 22.8, 21.2; IR (cm⁻¹): 3076, 2932, 2864, 1704, 1640, 1451, 1437, 1428, 1314, 1124, 993, 912, 613

FIG. 25 depicts the synthesis of (R)-2-(1-Methyl-2-oxocyclohexyl)acetaldehyde. To a round-bottom flask equipped with a magnetic stir were added 2-allyl-2-methylcyclohexan-1-one (80 mg, 0.526 mmol, 1.0 equiv) and CH₂Cl₂ (5.5 mL). The solution was cooled to −78° C. in an acetone/dry ice bath and ozone was bubbled through until the solution turned blue. The excess ozone was removed by bubbling oxygen thorough the solution until it turned clear. To the solution was added PPh₃ (275 mg, 1.05 mmol, 2 equiv) at −78° C. and the reaction mixture was allowed to warm to room temperature and the stirring was continued for 16 h. The solution was concentrated under the reduced pressure by rotary evaporation. Purification by flash column chromatography on silica gel (Et₂O/pentane=1:9 to 3:7) afforded the product (70 mg, 86%) as a colorless oil. R_{f}: 0.67 (EtOAc/Hex=2:8); [α]D²²: −64.52 (c=0.155, CH₂Cl₂); ¹H NMR (400 MHz, CDCl₃): 9.77 (t, J=2.1 Hz, 1H), 2.64-2.32 (m, 4H), 2.07-1.96 (m, 1H), 1.88-1.68 (m, 5H), 1.28 (s, 3H); ¹³C NMR (100 MHz, CDCl₃): 214.3, 201.5, 51.7, 47.8, 39.1, 38.4, 27.1, 23.7, 21.1; IR (cm⁻¹): 2934, 2862, 1703, 1462, 1448, 1431, 1178, 1159, 1128, 1080, 1042, 1014, 978, 934, 901, 868, 797, 735, 573; HRMS (m/z): calculated for C₉H₁₅O₂⁺: 155.1067; detected: 155.1069.

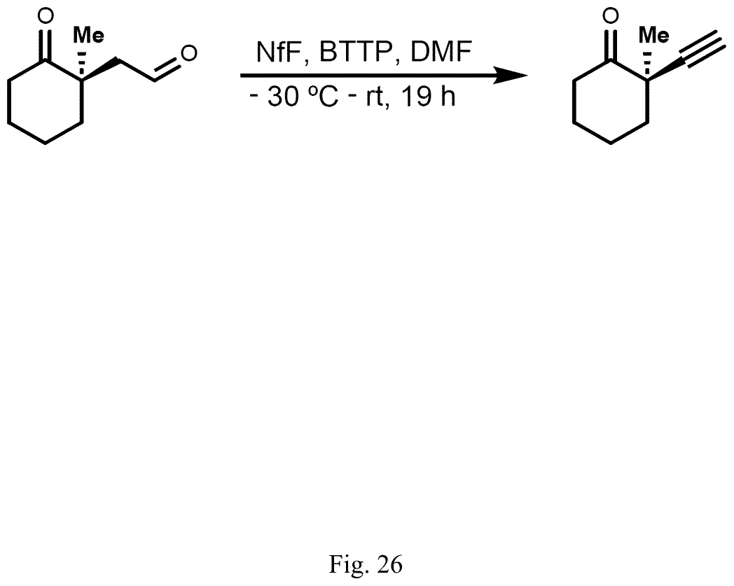

FIG. 26 depicts the synthesis of (R)-2-ethynyl-2-methylcyclohexan-1-one. For the synthesis of (R)-2-ethynyl-2-methylcyclohexan-1-one, procedure reported by Boltukhina et. al was applied [E. V. Boltukhina et al., Tetrahedron, 2011]. To a flame-dried round-bottom flask equipped with a magnetic stir bar were added 2-(1-methyl-2-oxocyclohexyl) acetaldehyde (309 mg, 2.00 mmol, 1 equiv), NfF (380 μL, 2.10 mmol, 1.05 equiv) and dry DMF (2 mL). The solution was cooled to −30° C. in an acetonitrile/dry ice bath and the BTTP base (3.68 mL, 12.02 mmol, 6 equiv) was added dropwise. The reaction mixture was allowed to warm to room temperature and the stirring was continued for 19 h. The reaction was quenched with saturated solution of NH₄Cl (15 mL) and extracted with Et₂O (3×15 mL). The organic solution was washed with water (4×15 mL) and brine (15 mL) and dried over anhydrous Na₂SO₄. The solution was concentrated under the reduced pressure by rotary evaporation. Purification by flash column chromatography on silica gel (Et₂O/pentane=1:99 to 3:97) afforded the product (212 mg, 78%) as a colorless oil. R_{f}: 0.48 (EtOAc/Hex=1:9); [α]D²²: 274.14 (c=0.116, CH₂Cl₂); ¹H NMR (400 MHz, CDCl₃): 3.01-2.90 (m, 1H), 2.38-2.25 (m, 2H), 2.16-2.03 (m, 3H), 1.78-1.49 (m, 3H), 1.31 (s, 3H); ¹³C NMR (100 MHz, CDCl₃): 208.8, 86.4, 72.7, 45.8, 42.1, 38.6, 28.2, 23.3, 22.4; IR (cm-1): 3290, 3271, 2982, 2936, 2864, 2112, 1717, 1462, 1448, 1427, 1375, 1333, 1312, 1277, 1258, 1232, 1155, 1121, 1111, 1090, 1063, 982, 905, 851, 829, 737, 688, 636, 569, 536, 519, 511, 498; HRMS (m/z): calculated for C₉H₁₃O⁺: 137.0961; detected: 137.0964.

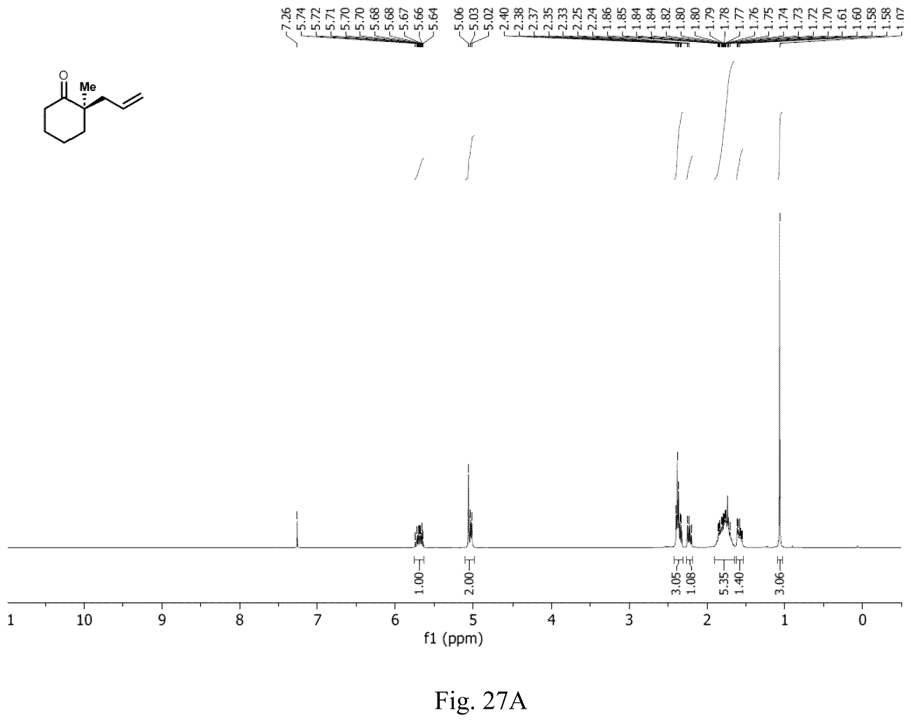

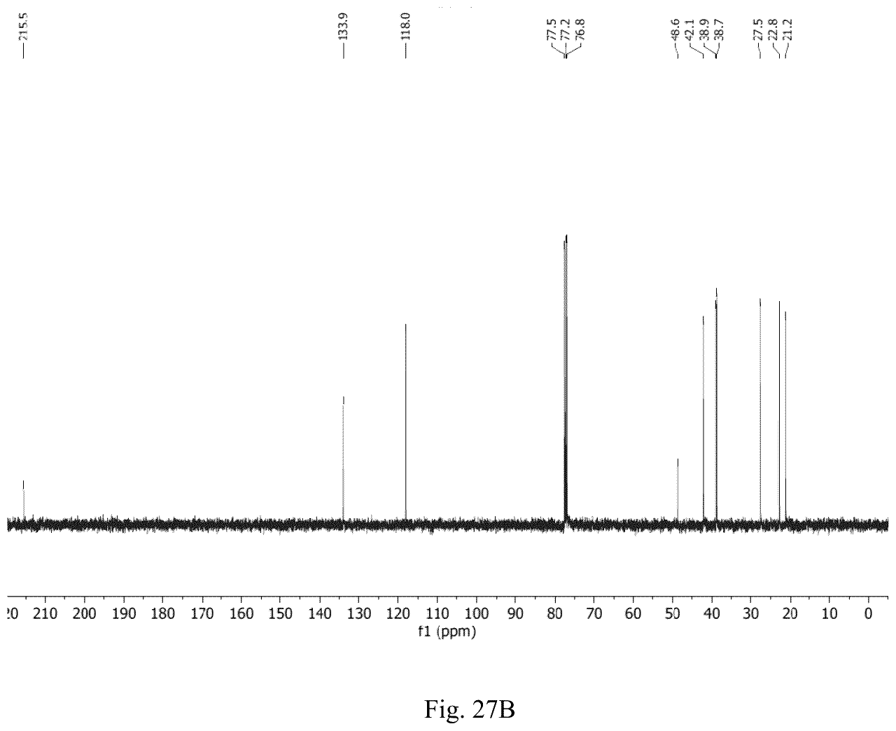

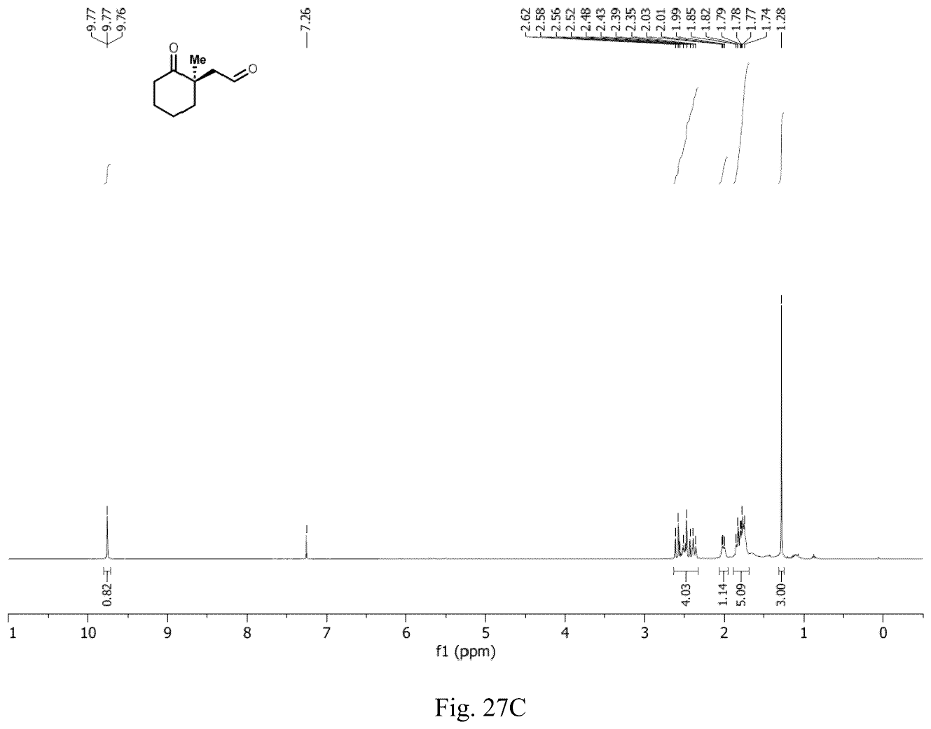

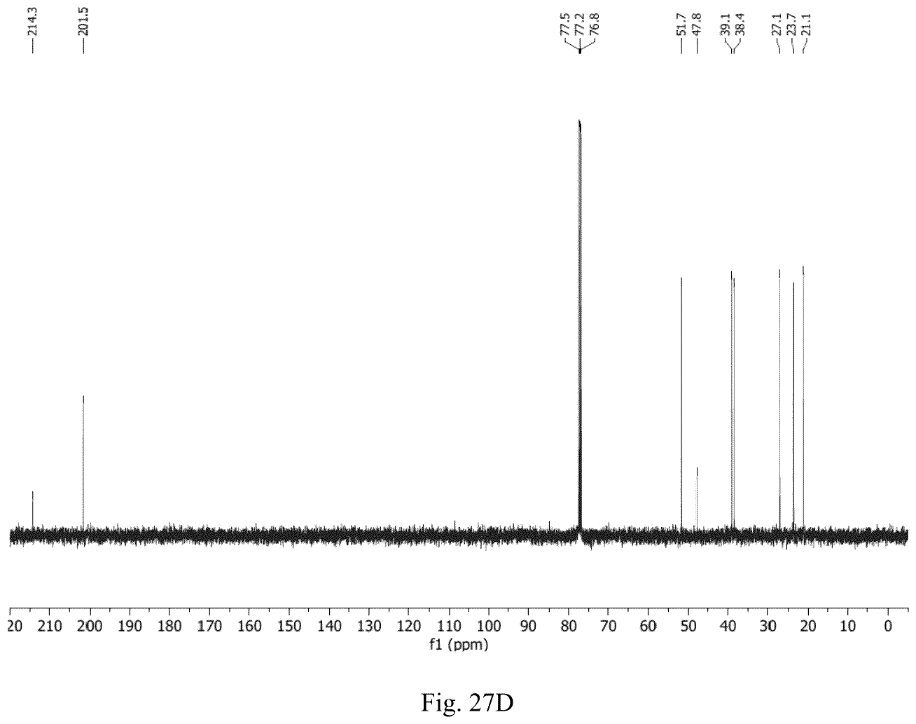

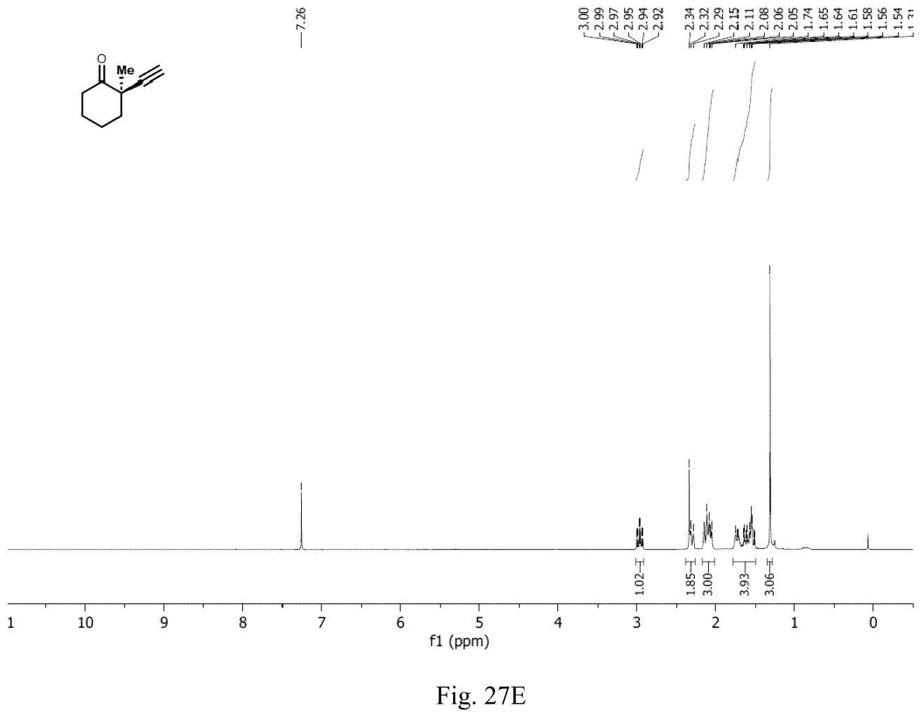

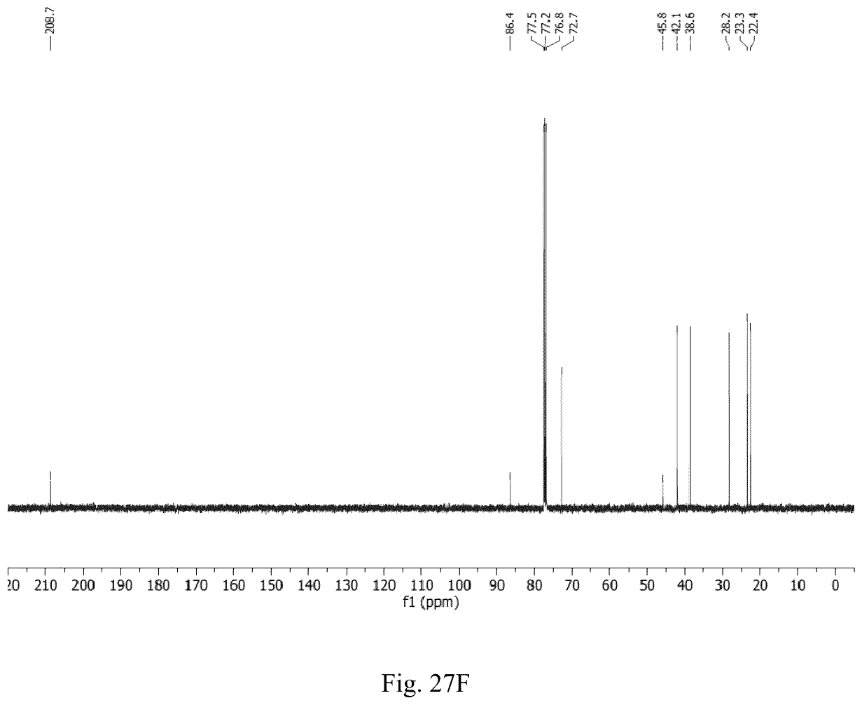

FIGS. 27A through 27F show the results for exemplary queries. FIGS. 27A and 27B are the queries for 1H and 13C NMR of (R)-2-Allyl-2-methylcyclohexan-1-one, respectively. ¹H NMR (400 MHz, CDCl₃): δ 5.75-5.63 (m, 1H), 5.10-4.98 (m, 2H), 2.43-2.31 (m, 3H), 2.27-2.18 (m, 1H), 1.91-1.65 (m, 5H), 1.63-1.54 (m, 1H), 1.07 (s, 3H); ¹³C NMR (100 MHz, CDCl₃): δ 215.5, 133.9, 118.0, 48.6, 42.1, 38.9, 38.7, 27.5, 22.8, 21.2.

FIGS. 27C and 27D are the queries for 1H and 13C NMR of ®-2-(1-Methyl-2-oxocyclohexyl)acetaldehyde, respectively. ¹H NMR (400 MHz, CDCl₃): δ 9.77 (t, J=2.1 Hz, 1H), 2.64-2.32 (m, 4H), 2.07-1.96 (m, 1H), 1.88-1.68 (m, 5H), 1.28 (s, 3H); ¹³C NMR (100 MHz, CDCl₃): δ 214.3, 201.5, 51.7, 47.8, 39.1, 38.4, 27.1, 23.7, 21.1.

FIGS. 27E and 27F are the queries for 1H and 13C NMR of ®-2-ethynyl-2-methylcyclohexan-1-one, respectively. ¹H NMR (400 MHz, CDCl₃): δ 3.01-2.90 (m, 1H), 2.38-2.25 (m, 2H), 2.16-2.03 (m, 3H), 1.78-1.49 (m, 3H), 1.31 (s, 3H); ¹³C NMR (100 MHz, CDCl₃): δ 208.8, 86.4, 72.7, 45.8, 42.1, 38.6, 28.2, 23.3, 22.4.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A system, comprising:
a transformer encoder with a compression layer;
a transformer decoder with an expansion layer;
the transformer encoder configured to transform one or more inputs into a control latent vector;
a noise injection element configured to add noise to the control latent vector to create a noisy latent vector;
a weighting element configured to add one or more weightings to the control latent vector to create an exact latent vector; and
the transformer decoder configured to transform the noisy latent vector and exact latent vector into an output.

2. The system of claim 1, wherein the one or more inputs is selected from one or more condition-scaled embedding vectors, one or more Simplified Molecular Input Line Entry System (SMILES) tokens, one or more SMILES Arbitrary Target Specification (SMARTS) tokens, one or more center-labelled products (CLP), reacting sites, reacting centers, or one or more reaction center labeled target molecules or compounds.

3. The system of claim 1, wherein the output is selected from one or more Simplified Molecular Input Line Entry System (SMILES) tokens, one or more SMILES Arbitrary Target Specification (SMARTS) tokens, one or more synthesis pathways, one or more retrosynthesis pathways, one or more labelled molecules or compounds, one or more templates, one or more reaction templates, one or more site-specific templates (SST).

4. The system of claim 1, further comprising one or more condition-scaled embedding vectors configured to attach one or more conditions to the output of the transformer decoder.

5. The system of claim 4, wherein the one or more conditioned-scaled embedding vectors are selected from molecule properties, SMILES tokens, positional embeddings, reacting sites, reaction centers, positional embedding for reacting sites or reaction centers, or molecular transformation sites.

6. The system of claim 1, wherein the transformer decoder is configured to pass the output through a linear layer, and softmax the output, to produce one or more output distribution probabilities.

7. The system of claim 1, wherein the transformer system is further configured to calculate a distance between a control latent vector used to generate a first output and a control latent vector used to generate a second output to produce a measured distance between the first and second outputs.

8. A method for retrosynthetic planning comprising:
providing one or more target molecules;
specifying one or more reaction centers on the one or more target molecules;
comparing the one or more target molecules to a database of reference reactions;
measuring a similarity between at least one of the one or more target molecules and a molecule in the reference reactions; and
generating one or more site-specific templates based on the measured similarity.

9. The system of claim 1, wherein the noise is gaussian noise.

10. The system of claim 1, wherein the transformer decoder and the latent space comprise a lambda-delta loss function.

11. The system of claim 1, wherein the transformer encoder is configured to accept one or more positional embedding inputs for reaction centers.

12. The system of claim 1, wherein the output comprises a reaction template.
